SEQUENCE LISTING (67 sequences)

SEQ ID NO: 1 - DNA, 54 bp, Artificial sequence
  Leader
atggactggacctggattctgttcctggtggccgccgccacaagggtgcacagc

SEQ ID NO: 2 - DNA, 462 bp, Artificial sequence
  Self-assembling polypeptide from lumazine synthase of Aquifex aeolicus
atgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcga

SEQ ID NO: 3 - DNA, 45 bp, Artificial sequence
  Linker
ggaggctccggaggatctggagggagtggaggctcaggaggaggc

SEQ ID NO: 4 - DNA, 516 bp, Artificial sequence
  Fragment of gp120
gacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatggaacaataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcaacagcacttggtttaattccacc

SEQ ID NO: 5 - DNA, 1083 bp, Artificial sequence
  Expressible nucleic acid sequence encoding antigen of SEQ ID NO 10
atggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcgacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatggaacaataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcaacagcacttggtttaattccacctgataa

SEQ ID NO: 6 - protein, 18 aa, Artificial sequence
  IgE leader sequence encoded by SEQ ID NO 1
MDWTWILFLVAAATRVHS

SEQ ID NO: 7 - protein, 154 aa, Artificial sequence
  Self-assembling polypeptide encoded by SEQ ID NO2
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLR

SEQ ID NO: 8 - protein, 15 aa, Artificial sequence
  Linker encoded by SEQ ID NO3
GGSGGSGGSGGSGGG

SEQ ID NO: 9 - protein, 172 aa, Artificial sequence
  Viral antigen encoded by SEQ ID NO4
DTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNST

SEQ ID NO: 10 - protein, 359 aa, Artificial sequence
  Antigen encoded by SEQ ID NO5
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNST

SEQ ID NO: 11 - DNA, 594 bp, Artificial sequence
  IgE-GLT1 ( IgE leader and a fragment of gp120)
ggatccgccaccatggactggacctggattctgttcctggtggccgccgccacaagggtgcacagcgacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatggaacaataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcaacagcacttggtttaattccacctgataactcgag

SEQ ID NO: 12 - protein, 190 aa, Artificial sequence
  IgE-gp120 encoded by SEQ ID NO11
MDWTWILFLVAAATRVHSDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNST

SEQ ID NO: 13 - DNA, 501 bp, Artificial sequence
  Self-assembling polypeptide 3BVE
gggctgagtaaggacattatcaagctgctgaacgaacaggtgaacaaagagatgcagtctagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaatgagaaccacgggctgtacctggcagatcagtatgtcaagggcatcgctaagtcaaggaaaagc

SEQ ID NO: 14 - DNA, 1032 bp, Artificial sequence
  Self-assembling polypeptide RBE
ctgagcattgcccccacactgattaaccgggacaaaccctacaccaaagaggaactgatggagattctgagactggctattatcgctgagctggacgccatcaacctgtacgagcagatggcccggtattctgaggacgagaatgtgcgcaagatcctgctggatgtggccagggaggagaaggcacacgtgggagagttcatggccctgctgctgaacctggaccccgagcaggtgaccgagctgaagggcggctttgaggaggtgaaggagctgacaggcatcgaggcccacatcaacgacaataagaaggaggagagcaacgtggagtatttcgagaagctgagatccgccctgctggatggcgtgaataagggcaggagcctgctgaagcacctgcctgtgaccaggatcgagggccagagcttcagagtggacatcatcaagtttgaggatggcgtgcgcgtggtgaagcaggagtacaagcccatccctctgctgaagaagaagttctacgtgggcatcagggagctgaacgacggcacctacgatgtgagcatcgccacaaaggccggcgagctgctggtgaaggacgaggagtccctggtcatccgcgagatcctgtctacagagggcatcaagaagatgaagctgagctcctgggacaatccagaggaggccctgaacgatctgatgaatgccctgcaggaggcatctaacgcaagcgccggaccattcggcctgatcatcaatcccaagagatacgccaagctgctgaagatctatgagaagtccggcaagatgctggtggaggtgctgaaggagatcttccggggcggcatcatcgtgaccctgaacatcgatgagaacaaagtgatcatctttgccaacacccctgccgtgctggacgtggtggtgggacaggatgtgacactgcaggagctgggaccagagggcgacgatgtggcctttctggtgtccgaggccatcggcatcaggatcaagaatccagaggcaatcgtggtgctggag

SEQ ID NO: 15 - DNA, 645 bp, Artificial sequence
  Self-assembling polypeptide I3
gagaaagcagccaaagcagaggaagcagcacggaagatggaagaactgttcaagaagcacaagatcgtggccgtgctgagggccaactccgtggaggaggccaagaagaaggccctggccgtgttcctgggcggcgtgcacctgatcgagatcacctttacagtgcccgacgccgataccgtgatcaaggagctgtctttcctgaaggagatgggagcaatcatcggagcaggaaccgtgacaagcgtggagcagtgcagaaaggccgtggagagcggcgccgagtttatcgtgtcccctcacctggacgaggagatctctcagttctgtaaggagaagggcgtgttttacatgccaggcgtgatgacccccacagagctggtgaaggccatgaagctgggccacacaatcctgaagctgttccctggcgaggtggtgggcccacagtttgtgaaggccatgaagggccccttccctaatgtgaagtttgtgcccaccggcggcgtgaacctggataacgtgtgcgagtggttcaaggcaggcgtgctggcagtgggcgtgggcagcgccctggtgaagggcacacccgtggaagtcgctgagaaggcaaaggcattcgtggaaaagattagggggtgtactgag

SEQ ID NO: 16 - DNA, 72 bp, Artificial sequence
  Linker
ggcggctctggcggaagtggcggaagtgggggaagtggaggcggcggaagcgggggaggcagcgggggaggg

SEQ ID NO: 17 - DNA, 27 bp, Artificial sequence
  Linker
ggcggaagcggcggaagcggcgggtct

SEQ ID NO: 18 - protein, 241 aa, Artificial sequence
  Rigid linker 4QTL-115 Angstroms, single chain 3-helix bundle
NEDDMKKLYKQMVQELEKARDRMEKLYKEMVELIQKAIELMRKIFQEVKQEVEKAIEEMKKLYDEAKKKIEQMIQQIKQGGDKQKMEELLKRAKEEMKKVKDKMEKLLEKLKQIMQEAKQKMEKLLKQLKEEMKKMKEKMEKLLKEMKQRMEEVKKKMDGDDELLEKIKKNIDDLKKIAEDLIKKAEENIKEAKKIAEQLVKRAKQLIEKAKQVAEELIKKILQLIEKAKEIAEKVLKGLE

SEQ ID NO: 19 - DNA, 36 bp, Artificial sequence
  Linker
ggcggcagcggcggcagcggcgggagcggaggaagt

SEQ ID NO: 20 - DNA, 1104 bp, Artificial sequence
  IgE-GLT1-3BVE
atggactggacctggattctgttcctggtggccgccgccacaagggtgcacagcgacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatggaacaataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcaacagcacttggtttaattccaccggcggaagcggcggaagcggcgggtctgggctgagtaaggacattatcaagctgctgaacgaacaggtgaacaaagagatgcagtctagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaatgagaaccacgggctgtacctggcagatcagtatgtcaagggcatcgctaagtcaaggaaaagctgataa

SEQ ID NO: 21 - protein, 366 aa, Artificial sequence
  IgE-GLT1-3BVE
MDWTWILFLVAAATRVHSDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNSTGGSGGSGGSGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 22 - protein, 9 aa, Artificial sequence
  Linker
GGSGGSGGS

SEQ ID NO: 23 - protein, 167 aa, Artificial sequence
  3BVE scaffold
GLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 24 - DNA, 1257 bp, Artificial sequence
  IgE-GLT1-I3
atggactggacctggattctgttcctggtggccgccgccacaagggtgcacagcgacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatggaacaataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcaacagcacttggtttaattccaccggcggcagcggcggcagcggcgggagcggaggaagtgagaaagcagccaaagcagaggaagcagcacggaagatggaagaactgttcaagaagcacaagatcgtggccgtgctgagggccaactccgtggaggaggccaagaagaaggccctggccgtgttcctgggcggcgtgcacctgatcgagatcacctttacagtgcccgacgccgataccgtgatcaaggagctgtctttcctgaaggagatgggagcaatcatcggagcaggaaccgtgacaagcgtggagcagtgcagaaaggccgtggagagcggcgccgagtttatcgtgtcccctcacctggacgaggagatctctcagttctgtaaggagaagggcgtgttttacatgccaggcgtgatgacccccacagagctggtgaaggccatgaagctgggccacacaatcctgaagctgttccctggcgaggtggtgggcccacagtttgtgaaggccatgaagggccccttccctaatgtgaagtttgtgcccaccggcggcgtgaacctggataacgtgtgcgagtggttcaaggcaggcgtgctggcagtgggcgtgggcagcgccctggtgaagggcacacccgtggaagtcgctgagaaggcaaaggcattcgtggaaaagattagggggtgtactgagtgataa

SEQ ID NO: 25 - protein, 417 aa, Artificial sequence
  IgE-GLT1-I3
MDWTWILFLVAAATRVHSDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNSTGGSGGSGGSGGSEKAAKAEEAARKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 26 - protein, 215 aa, Artificial sequence
  I3 scaffold
EKAAKAEEAARKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 27 - protein, 12 aa, Artificial sequence
  Linker
GGSGGSGGSGGS

SEQ ID NO: 28 - DNA, 1674 bp, Artificial sequence
  IgE-GLT1-RBE
atggactggacctggattctgttcctggtggccgccgccacaagggtgcacagcctgagcattgcccccacactgattaaccgggacaaaccctacaccaaagaggaactgatggagattctgagactggctattatcgctgagctggacgccatcaacctgtacgagcagatggcccggtattctgaggacgagaatgtgcgcaagatcctgctggatgtggccagggaggagaaggcacacgtgggagagttcatggccctgctgctgaacctggaccccgagcaggtgaccgagctgaagggcggctttgaggaggtgaaggagctgacaggcatcgaggcccacatcaacgacaataagaaggaggagagcaacgtggagtatttcgagaagctgagatccgccctgctggatggcgtgaataagggcaggagcctgctgaagcacctgcctgtgaccaggatcgagggccagagcttcagagtggacatcatcaagtttgaggatggcgtgcgcgtggtgaagcaggagtacaagcccatccctctgctgaagaagaagttctacgtgggcatcagggagctgaacgacggcacctacgatgtgagcatcgccacaaaggccggcgagctgctggtgaaggacgaggagtccctggtcatccgcgagatcctgtctacagagggcatcaagaagatgaagctgagctcctgggacaatccagaggaggccctgaacgatctgatgaatgccctgcaggaggcatctaacgcaagcgccggaccattcggcctgatcatcaatcccaagagatacgccaagctgctgaagatctatgagaagtccggcaagatgctggtggaggtgctgaaggagatcttccggggcggcatcatcgtgaccctgaacatcgatgagaacaaagtgatcatctttgccaacacccctgccgtgctggacgtggtggtgggacaggatgtgacactgcaggagctgggaccagagggcgacgatgtggcctttctggtgtccgaggccatcggcatcaggatcaagaatccagaggcaatcgtggtgctggagggcggctctggcggaagtggcggaagtgggggaagtggaggcggcggaagcgggggaggcagcgggggaggggacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatggaacaataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcaacagcacttggtttaattccacc

SEQ ID NO: 29 - DNA, 72 bp, Artificial sequence; identical to SEQ ID NO: 16
  Linker

SEQ ID NO: 30 - protein, 558 aa, Artificial sequence
  IgE leader-RBE-linker-HIV antigen
MDWTWILFLVAAATRVHSLSIAPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLEGGSGGSGGSGGSGGGGSGGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNST

SEQ ID NO: 31 - protein, 344 aa, Artificial sequence
  RBE scaffold
LSIAPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLE

SEQ ID NO: 32 - protein, 24 aa, Artificial sequence
  Linker
GGSGGSGGSGGSGGGGSGGGSGGG

SEQ ID NO: 33 - DNA, 1254 bp, Artificial sequence
  NivFtop_stab2_gMax_Nt_60mer
atggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcggggtcacttgtgccggacgagccatcggaaatgctaccgccgcccagattactgccggagtcgccctgtatgaagccatgaagaatgccgacaacatcaataagctgaagagctccatcgagagcaccaacgaggccgtggtgaagctgcaggagacagccgagaagacagtgtacgtgctgacagccctgcaggactatatcaacaccaatctggtgcccacaatcgataagatcagctgcaagcagaccgaggcatccctggacgccgccctgtccaagtacctgtctgatctgctgtacgtgttcggccccaacctgagcgaccccgtgagcaattctatgcctatccaggccatctctcaggccttcggcggcaactacagcaccctgctgaggacactgggctatgccccagaggactttgacgatctgctggagagcgattccatcacaggccagatcatctacgtggacctgtctagctactatatcatcgtgagagtgtattttccaaatggctccggccccctgaccaaggatatcgtgatcaagatgatccccaacgtgtctaatatgagccagtgtacaggctctgtgatggagaactacaagaccaggctgaatggcatcctgacacctatcaagggcgccctggagatctataagaataactgtcacgatggatgataa

SEQ ID NO: 34 - DNA, 693 bp, Artificial sequence
  NivFtop_stab2_gMax
ggggtcacttgtgccggacgagccatcggaaatgctaccgccgcccagattactgccggagtcgccctgtatgaagccatgaagaatgccgacaacatcaataagctgaagagctccatcgagagcaccaacgaggccgtggtgaagctgcaggagacagccgagaagacagtgtacgtgctgacagccctgcaggactatatcaacaccaatctggtgcccacaatcgataagatcagctgcaagcagaccgaggcatccctggacgccgccctgtccaagtacctgtctgatctgctgtacgtgttcggccccaacctgagcgaccccgtgagcaattctatgcctatccaggccatctctcaggccttcggcggcaactacagcaccctgctgaggacactgggctatgccccagaggactttgacgatctgctggagagcgattccatcacaggccagatcatctacgtggacctgtctagctactatatcatcgtgagagtgtattttccaaatggctccggccccctgaccaaggatatcgtgatcaagatgatccccaacgtgtctaatatgagccagtgtacaggctctgtgatggagaactacaagaccaggctgaatggcatcctgacacctatcaagggcgccctggagatctataagaataactgtcacgatggatgataa

SEQ ID NO: 35 - protein, 416 aa, Artificial sequence
  IgE-Self Assembly-Linker-Viral Antigen
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGGVTCAGRAIGNATAAQITAGVALYEAMKNADNINKLKSSIESTNEAVVKLQETAEKTVYVLTALQDYINTNLVPTIDKISCKQTEASLDAALSKYLSDLLYVFGPNLSDPVSNSMPIQAISQAFGGNYSTLLRTLGYAPEDFDDLLESDSITGQIIYVDLSSYYIIVRVYFPNGSGPLTKDIVIKMIPNVSNMSQCTGSVMENYKTRLNGILTPIKGALEIYKNNCHDG

SEQ ID NO: 36 - protein, 229 aa, Artificial sequence
  NivFtop_stab2_gMax
GVTCAGRAIGNATAAQITAGVALYEAMKNADNINKLKSSIESTNEAVVKLQETAEKTVYVLTALQDYINTNLVPTIDKISCKQTEASLDAALSKYLSDLLYVFGPNLSDPVSNSMPIQAISQAFGGNYSTLLRTLGYAPEDFDDLLESDSITGQIIYVDLSSYYIIVRVYFPNGSGPLTKDIVIKMIPNVSNMSQCTGSVMENYKTRLNGILTPIKGALEIYKNNCHDG

SEQ ID NO: 37 - DNA, 1254 bp, Artificial sequence
  NivFtop_stab2_gMax_Ct_60mer
atggactggacctggattctgttcctggtggccgccgccacaagggtgcacagcggggtcacttgtgccggacgagccatcggaaatgctaccgccgcccagattactgccggagtcgccctgtatgaagccatgaagaatgccgacaacatcaataagctgaagagctccatcgagagcaccaacgaggccgtggtgaagctgcaggagacagccgagaagacagtgtacgtgctgacagccctgcaggactatatcaacaccaatctggtgcccacaatcgataagatcagctgcaagcagaccgaggcatccctggacgccgccctgtccaagtacctgtctgatctgctgtacgtgttcggccccaacctgagcgaccccgtgagcaattctatgcctatccaggccatctctcaggccttcggcggcaactacagcaccctgctgaggacactgggctatgccccagaggactttgacgatctgctggagagcgattccatcacaggccagatcatctacgtggacctgtctagctactatatcatcgtgagagtgtattttccaaatggctccggccccctgaccaaggatatcgtgatcaagatgatccccaacgtgtctaatatgagccagtgtacaggctctgtgatggagaactacaagaccaggctgaatggcatcctgacacctatcaagggcgccctggagatctataagaataactgtcacgatggaggaggctccggaggatctggagggagtggaggctcaggaggaggcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgatgataa

SEQ ID NO: 38 - protein, 416 aa, Artificial sequence
  NivFtop_stab2_gMax_Ct_60mer
MDWTWILFLVAAATRVHSGVTCAGRAIGNATAAQITAGVALYEAMKNADNINKLKSSIESTNEAVVKLQETAEKTVYVLTALQDYINTNLVPTIDKISCKQTEASLDAALSKYLSDLLYVFGPNLSDPVSNSMPIQAISQAFGGNYSTLLRTLGYAPEDFDDLLESDSITGQIIYVDLSSYYIIVRVYFPNGSGPLTKDIVIKMIPNVSNMSQCTGSVMENYKTRLNGILTPIKGALEIYKNNCHDGGGSGGSGGSGGSGGGMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLR

SEQ ID NO: 39 - DNA, 132 bp, Artificial sequence
  Leader
atggactggacctggagaatcctgttcctggtggccgccgccaccggcacacacgccgatacacacttccccatctgcatcttttgctgtggctgttgccataggtccaagtgtgggatgtgctgcaaaact

SEQ ID NO: 40 - protein, 19 aa, Artificial sequence
  Leader
MDWTWRILFLVAAATGTHA

SEQ ID NO: 41 - DNA, 315 bp, West Nile Virus
atgtctaagaaaccaggagggcccggcaagagccgggctgtcaatatgctaaaacgcggaatgccccgcgtgttgtccttgattggactgaagagggctatgttgaacctgatcgacggcaaggggccaatacgatttgtgttggctctcttggcgttcttcaggttcacagcaattgctccgacccgagcagtgctggatcgatggagaggtgtgaacaaacaaacagcgatgaaacaccttttgagttttaagaaggaactagggaccttgaccagtgctatcaatcggcggagctcaaaacaaaagaaaaga

SEQ ID NO: 42 - DNA, 1518 bp, Human papillomavirus
atgtcactttggcttccatcagaagctactgtttaccttccaccagttccagtttcaaaagttgtttcaactgatgaatacgttgctaggactaatatttactaccatgctggaacttcaaggcttcttgctgttggacatccatactttccaattaaaaaaccaaataataataaaattcttgttccaaaagtttcaggacttcaatacagggtttttaggattcatcttccagatccaaataaatttggatttccagatacttcattttacaatccagatactcaaaggcttgtttgggcttgtgttggagttgaagttggaaggggacaaccacttggagttggaatttcaggacatccacttcttaataaacttgatgatactgaaaatgcttcagcttacgctgctaatgctggagttgataatagggaatgtatttcaatggattacaaacaaactcaactttgtcttattggatgtaaaccaccaattggagaacattggggaaaaggatcaccatgtactaatgttgctgttaatccaggagattgtccaccacttgaacttattaatactgttattcaagatggagatatggttgatactggatttggagctatggattttactactcttcaagctaataaatcagaagttccacttgatatctgtacttcaatttgtaaatacccagattacattaaaatggtttcagaaccatacggagattcactttttttttaccttaggagggaacaaatgtttgttaggcatctttttaatagggctggagctgttggagaaaatgttccagatgatctttacattaaaggatcaggatcaactgctaatcttgcttcatcaaattactttccaactccatcaggatcaatggttacttcagatgctcaaatttttaataaaccatactggcttcaaagggctcaaggacataataatggaatttgttggggaaatcaactttttgttactgttgttgatactactaggtcaactaatatgtcactttgtgctgctatttcaacttcagaaactacttacaaaaatactaattttaaagaataccttaggcatggagaagaatacgatcttcaatttatttttcaactttgtaaaattactcttactgctgatgttatgacttacattcattcaatgaattcaactattcttgaagattggaattttggacttcaaccaccaccaggaggaactcttgaagatacttacaggtttgttacttcacaagctattgcttgtcaaaaacatactccaccagctccaaaagaggatccacttaaaaaatacactttttgggaagttaatcttaaagaaaaattttcagcagatcttgatcaatttccacttggaaggaaatttcttcttcaagctggacttaaagctaaaccaaaatttactcttggaaaaaggaaagctactccaactacttcatcaacttcaactactgctaaaaggaaaaaaaggaaactttga

SEQ ID NO: 43 - DNA, 1422 bp, Human papillomavirus
atgaggcacaagaggagcgccaagaggaccaagagggccagcgccacccagctgtacaagacctgcaagcaggccggcacctgcccccccgacatcatccccaaggtggagggcaagaccatcgccgaccagatcctgcagtacggcagcatgggcgtgttcttcggcggcctgggcatcggcaccggcagcggcaccggcggcaggaccggctacatccccctgggcaccaggccccccaccgccaccgacaccctggcccccgtgaggccccccctgaccgtggaccccgtgggccccagcgaccccagcatcgtgagcctggtggaggagaccagcttcatcgacgccggcgcccccaccagcgtgcccagcatcccccccgacgtgagcggcttcagcatcaccaccagcaccgacaccacccccgccatcctggacatcaacaacaccgtgaccaccgtgaccacccacaacaaccccaccttcaccgaccccagcgtgctgcagccccccacccccgccgagaccggcggccacttcaccctgagcagcagcaccatcagcacccacaactacgaggagatccccatggacaccttcatcgtgagcaccaaccccaacaccgtgaccagcagcacccccatccccggcagcaggcccgtggccaggctgggcctgtacagcaggaccacccagcaggtgaaggtggtggaccccgccttcgtgaccacccccaccaagctgatcacctacgacaaccccgcctacgagggcatcgacgtggacaacaccctgtacttcagcagcaacgacaacagcatcaacatcgcccccgaccccgacttcctggacatcgtggccctgcacaggcccgccctgaccagcaggaggaccggcatcaggtacagcaggatcggcaacaagcagaccctgaggaccaggagcggcaagagcatcggcgccaaggtgcactactactacgacctgagcaccatcgaccccgccgaggagatcgagctgcagaccatcacccccagcacctacaccaccaccagccacgccgccagccccaccagcatcaacaacggcctgtacgacatctacgccgacgacttcatcaccgacaccagcaccacccccgtgcccagcgtgcccagcaccagcctgagcggctacatccccgccaacaccaccatccccttcggtggcgcctacaacatccccctggtgagcggccccgacatccccatcaacatcaccgaccaggcccccagcctgatccccatcgtgcccggcagcccccagtacaccatcatcgccgacgccggcgacttctacctgcaccccagctactacatgctgaggaagaggaggaagaggctgccctacttcttcagcgacgtgagcctggccgcctga

SEQ ID NO: 44 - DNA, 1725 bp, Respiratory syncytial virus
gagctgcccatcctgaaaacaaacgccatcaccaccatcctggccgccgtgaccctgtgcttcgccagcagccagaacatcaccgaggaattctaccagagcacctgtagcgccgtgtccaagggctacctgtctgccctgcggaccggctggtacaccagcgtgatcaccatcgagctgagcaacatcaaagaaaacaagtgcaacggcaccgacgccaaagtgaagctgatcaagcaggaactggacaagtacaagaacgccgtgaccgagctgcagctgctgatgcagagcacccctgccgccaacaacagagccagacgcgagctgccccggttcatgaactacaccctgaacaacaccaagaacaccaacgtgaccctgagcaagaagcggaagcggcggttcctgggattcctgctgggcgtgggcagcgccattgcctctggaatcgctgtgtctaaggtgctgcacctggaaggcgaagtgaacaagatcaagtccgccctgctgagcaccaacaaggccgtggtgtccctgagcaacggcgtgtccgtgctgaccagcaaggtgctggatctgaagaactacatcgacaagcagctgctgcctatcgtgaacaagcagagctgcagcatcagcaacatcgagacagtgatcgagttccagcagaagaacaaccggctgctggaaatcacccgcgagttcagcgtgaacgccggcgtgaccacccccgtgtccacctacatgctgaccaacagcgagctgctgagcctgatcaacgacatgcccatcaccaacgaccagaaaaagctgatgagcaacaacgtgcagatcgtgcggcagcagagctactccatcatgtccatcatcaaagaagaggtgctggcctacgtggtgcagctgcccctgtacggcgtgatcgacaccccctgctggaagctgcacaccagccccctgtgcaccaccaacaccaaagagggcagcaacatctgcctgacccggaccgaccggggctggtactgcgataatgccggcagcgtgtcattctttccacaggccgagacatgcaaggtgcagagcaaccgggtgttctgcgacaccatgaacagcctgaccctgccctccgaagtgaacctgtgcaacatcgacatcttcaaccctaagtacgactgcaagatcatgacctccaagaccgacgtgtccagctccgtgatcacctccctgggcgccatcgtgtcctgctacggcaagaccaagtgcaccgccagcaacaagaaccggggcatcatcaagaccttcagcaacggctgcgactacgtgtccaacaagggggtggacaccgtgtccgtgggcaacaccctgtactacgtgaacaaacaggaaggcaagagcctgtacgtgaagggcgagcccatcatcaacttctacgaccccctggtgttccccagcgacgagttcgacgccagcatcagccaggtgaacgagaagatcaaccagagcctggccttcatcagaaagagcgacgagctgctgcacaatgtgaatgccggcaagagcaccaccaatatcatgatcaccacaatcatcatcgtgatcattgtgatcctgctgtccctgatcgccgtgggcctgctgctgtactgcaaggcccggtccacccctgtgaccctgtccaaggaccagctgagcggaatcatcaacaatatcgccttctccaactga

SEQ ID NO: 45 - protein, 479 aa, Respiratory syncytial virus
  misc_feature (247)..(247): Xaa can be any naturally occurring amino acid
  misc_feature (259)..(259): Xaa can be any naturally occurring amino acid
  misc_feature (299)..(299): Xaa can be any naturally occurring amino acid
MQSTPAANNRARRELPRFMNYTLNNTKNTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYXDNAGSVSFFPQXETCKVQSNRVFCDTMNSLTLPSEVNLCNIDIFNPKYDCKXMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGIINNIAFSN

SEQ ID NO: 46 - DNA, 897 bp, Respiratory syncytial virus
atgtccaagaataaggatcagaggaccgcgaaaacgcttgagaggacgtgggacacgctgaaccacctcctgttcatctcctcgtgtctctacaagctcaaccttaagtccatcgcgcagatcaccttgagcattctcgccatgatcatctccaccagccttatcattgccgcaatcatcttcatcgcatccgccaaccataaggtgacattgactacagcgattatccaagacgctactagccagatcaagaataccacgccgacctatttgacgcaaaatcctcagttgggaattagcttctcgaatctctcggaaaccacgtcgcagccgactacaattcttgcgtcaacgactccatcggccaaatcaacaccacaatcgactaccgtaaaaacgaagaacacgactacaacacagattcagccttcaaagcccacgaccaaacagagacagaataagccgcccaacaagcccaacaatgattttcacttcgaggtgtttaacttcgtgccctgttcgatttgcagcaataaccccacgtgctgggcgatttgcaagcgaatcccgaataagaagcccgggaaaaagaccacgacgaaaccgacaaagaagccgacaatcaagacaacgaaaaaggatcttaaacctcagacgacaaagcctaaggaagtcttgacaacgaagcctacggaaaaacccactatcaatactaccaagactaacatccggacaacactgctgacgagcaataccacgggaaacccggagctcacatcgcagaaagagacactccattcgacatcctccgagggtaacccttcgcccagccaggtgtatacgacgtcagaataccctagccaaccctcatcgccctcaaatacgacccggcaatga

SEQ ID NO: 47 - protein, 298 aa, Respiratory syncytial virus
MSKNKDQRTAKTLERTWDTLNHLLFISSCLYKLNLKSIAQITLSILAMIISTSLIIAAIIFIASANHKVTLTTAIIQDATSQIKNTTPTYLTQNPQLGISFSNLSETTSQPTTILASTTPSAKSTPQSTTVKTKNTTTTQIQPSKPTTKQRQNKPPNKPNNDFHFEVFNFVPCSICSNNPTCWAICKRIPNKKPGKKTTTKPTKKPTIKTTKKDLKPQTTKPKEVLTTKPTEKPTINTTKTNIRTTLLTSNTTGNPELTSQKETLHSTSSEGNPSPSQVYTTSEYPSQPSSPSNTTRQ

SEQ ID NO: 48 - DNA, 879 bp, Respiratory syncytial virus
  misc_feature (37)..(37): n is a, c, g, or t
  misc_feature (173)..(173): n is a, c, g, or t
  misc_feature (285)..(285): n is a, c, g, or t
atgagcaaaaacaaaaaccaaaggacggctcggacgnttgagaaaacatgggacacgcttaatcaccttattgtgatctcatcgtgtttgtaccggttgaatctcaagagcatcgcccagattgcgctgtcagtcctggccatgattatctcgacatcactcatcatcgcagncatcatctttatcatttcagcgaatcacaaggtaacgcttacaacagtcacggtgcagaccatcaagaatcataccgaaaagaatatcacaacctacctcacccaagtcagnccggagagagtaagcccctcaaaacagcctactacgacacctcccatccacacgaactcggcgaccatctcaccgaataccaaatcagaaacgcatcatacgaccgcacagacaaagggacgaaccactacacccacacagaacaacaaacccagcaccaagccgaggccaaagaatccgcccaagaagccgaaagatgactatcactttgaagtgttcaacttcgtaccgtgttcgatttgcgggaataatcagttgtgcaaatccatttgcaagacgatcccatccaacaaaccgaagaagaaacctaccatcaagcccacaaacaagccaacgacaaaaacaacgaacaagcgcgatcccaaaacgctcgcgaaaacgttgaagaaggaaacgacgacaaaccctacgaagaaacccacgcccaagaccactgagagagacacctccacctcgcaatcgacggtacttgacacgactacgagcaagcacactatccagcaacagtccctgcactcaaccacgcccgagaatacaccaaactcaacacagactccgacagcttcagagccttccacttcgaattccacatga

SEQ ID NO: 49 - protein, 292 aa, Respiratory syncytial virus
  misc_feature (13)..(13): Xaa can be any naturally occurring amino acid
  misc_feature (20)..(20): Xaa can be any naturally occurring amino acid
  misc_feature (58)..(58): Xaa can be any naturally occurring amino acid
  misc_feature (95)..(95): Xaa can be any naturally occurring amino acid
MSKNKNQRTARTXEKTWDTXNHLIVISSCLYRLNLKSIAQIALSVLAMIISTSLIIAXIIFIISANHKVTLTTVTVQTIKNHTEKNITTYLTQVXPERVSPSKQPTTTPPIHTNSATISPNTKSETHHTTAQTKGRTTTPTQNNKPSTKPRPKNPPKKPKDDYHFEVFNFVPCSICGNNQLCKSICKTIPSNKPKKKPTIKPTNKPTTKTTNKRDPKTLAKTLKKETTTNPTKKPTPKTTERDTSTSQSTVLDTTTSKHTIQQQSLHSTTPENTPNSTQTPTASEPSTSNST

SEQ ID NO: 50 - DNA, 2034 bp, Zaire ebolavirus
  misc_feature (675)..(675): n is a, c, g, or t
  misc_feature (1492)..(1492): n is a, c, g, or t
  misc_feature (1752)..(1752): n is a, c, g, or t
  misc_feature (1802)..(1802): n is a, c, g, or t
atgggggtcactgggattctgcagctgcctagagatcgcttcaagcgaacctctttctttctgtgggtcatcattctgttccagaggacttttagtatccctctgggcgtcattcacaattctaccctgcaggtgagtgacgtcgataagctggtgtgtcgggacaaactgagctccaccaaccagctgagatctgtcggcctgaatctggaggggaacggagtggctaccgatgtcccaagtgcaacaaagagatgggggtttcgctcaggagtgccccctaaagtggtcaattacgaggccggggaatgggctgagaattgctataacctggaaatcaagaaacccgacggatcagagtgtctgccagccgctcccgatgggattcgcggattccctagatgcagatacgtgcacaaggtcagcggcaccgggccatgtgcaggagacttcgcctttcataaagaaggcgccttctttctgtacgatagactggcttccaccgtgatctatagggggaccacattcgccgagggagtggtcgcttttctgattctgcctcaggccaagaaagacttcttttctagtcatcctctgcgggaaccagtgaacgctaccgaggaccccagcagcggctactattccactaccatcagataccaggccacaggattnggcaccaatgagacagaatacctgtttgaagtggacaacctgacatatgtccagctggagtctaggttcactccccagtttctgctgcagctgaatgaaactatctataccagtggcaagcgctcaaatacaactgggaagctgatttggaaagtgaaccctgagatcgataccacaattggcgaatgggccttttgggagaccaagaaaaacctgacacggaagatcagaagcgaggaactgtccttcaccgcagtgagtaatagggccaaaaacatttcaggccagagcccagcacgaacttcctctgaccccgggaccaatactaccacagaagatcacaagatcatggccagcgagaacagttcagctatggtgcaggtccactcccagggaagggaggcagccgtgtctcatctgactaccctggccacaatctctactagtccccagagccccacaactaagcccgggcctgacaatagcacccataacacacctgtgtacaaactggatatctccgaagccacccaggtcgagcagcaccatcggagaacagacaatgattccactgcatctgacacccctccagcaaccacagctgcaggaccccccaaggctgagaatactaacaccagcaaaagcaccgacctgctggaccccgcaactaccacatcaccacagaaccacagcgagacagccgggaacaataacactcaccatcaggacaccggagaggaatccgccagctccggcaagctggggctgatcacaaatactattgctggagtggcaggactgatcacaggcgggaggngaactcgacgagaagctattgtgaacgcacagcccaaatgcaatcctaacctgcactattggactacccaggacgagggagcagctatcggactggcatggattccatactttgggcccgcagccgaaggaatctataccgagggcctgatgcataatcaggatggactgatctgtggcctgcggcagctggctaacgaaacaactcaggcactgcagctgttcctgcgagctaccacagagctgcggacctttagcatnctgaatcgcaaggcaattgacttcctgctgcagcgatggggaggcacatnccacatcctgggaccagactgctgtattgagcctcatgattggacaaagaacatcactgacaaaattgatcagatcattcacgacttcgtggataaaacactgccagatcagggggacaatgataactggtggactggatggagacagtggattcccgccggcattggcgtcaccggcgtcattattgccgtcattgctctgttctgtatttgtaagttcgtgttctgataa

SEQ ID NO: 51 - protein, 676 aa, Zaire ebolavirus
  misc_feature (225)..(225): Xaa can be any naturally occurring amino acid
  misc_feature (498)..(498): Xaa can be any naturally occurring amino acid
  misc_feature (584)..(584): Xaa can be any naturally occurring amino acid
  misc_feature (601)..(601): Xaa can be any naturally occurring amino acid
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGXGTNETEYLFEVDNLTYVQLESRFTPQFLLQLNETIYTSGKRSNTTGKLIWKVNPEIDTTIGEWAFWETKKNLTRKIRSEELSFTAVSNRAKNISGQSPARTSSDPGTNTTTEDHKIMASENSSAMVQVHSQGREAAVSHLTTLATISTSPQSPTTKPGPDNSTHNTPVYKLDISEATQVEQHHRRTDNDSTASDTPPATTAAGPPKAENTNTSKSTDLLDPATTTSPQNHSETAGNNNTHHQDTGEESASSGKLGLITNTIAGVAGLITGGRXTRREAIVNAQPKCNPNLHYWTTQDEGAAIGLAWIPYFGPAAEGIYTEGLMHNQDGLICGLRQLANETTQALQLFLRATTELRTFSXLNRKAIDFLLQRWGGTXHILGPDCCIEPHDWTKNITDKIDQIIHDFVDKTLPDQGDNDNWWTGWRQWIPAGIGVTGVIIAVIALFCICKFVF

SEQ ID NO: 52 - DNA, 2034 bp, Sudan Ebolavirus
  misc_feature (202)..(202): n is a, c, g, or t
  misc_feature (422)..(422): n is a, c, g, or t
  misc_feature (510)..(510): n is a, c, g, or t
  misc_feature (720)..(720): n is a, c, g, or t
  misc_feature (1042)..(1042): n is a, c, g, or t
atggagggactgtcactgctgcagctgcctagagataagttcaggaaaagctccttctttgtgtgggtcatcattctgttccagaaggccttttcaatgcccctgggcgtggtcactaatagcaccctggaagtgacagagatcgatcagctggtctgtaaggaccacctggcttcaactgatcagctgaaaagcgtggggntgaacctggagggatcaggcgtcagcactgatattccttctgcaaccaagagatggggatttcgcagcggagtgccccctaaagtggtctcctacgaagcaggggagtgggccgaaaattgctataacctggagatcaagaaaccagatggcagcgaatgtctgccaccccctccagacggggtgcgcggattccccagatgcagatacgtccacaaggnccaggggaccggaccttgtccaggagactatgcctttcataaagatggcgctttctttctgtacgaccgcctggctagtacagtgatntatcgaggcgtcaatttcgccgagggcgtgatcgcttttctgattctggcaaagccaaaagaaaccttcctgcagagccctcccattagggaggccgtgaactacacagaaaacacttctagttactacgctacatcctacctggagtatgaaatcgagaactttggcgctcagcactctaccacactgttcaagattaacaataacacntttgtgctgctggatcgccctcatacaccacagttcctgtttcagctgaacgacactatccacctgcatcagcagctgagcaatactaccggaaaactgatttggacactggacgctaatatcaacgcagatattggcgagtgggccttctgggaaaataagaaaaacctgtccgagcagctgcggggagaggaactgagctttgaaacactgtccctgaatgaaactgaggacgatgacgccacctcaagccgaacaactaagggccggatctctgatcgggctaccagaaagtacagtgatctggtgccaaaagactctnccggcatggtgagtctgcacgtccctgaaggggagaccacactgccatcccagaactctactgagggccggagagtggacgtcaatacccaggagactatcaccgaaactaccgcaacaatcattggcactaacgggaataacatgcagatcagcaccattggcacagggctgtcctctagtcagattctgtcaagctcccctaccatggccccctcccctgagacacagacttctacaacttatacacccaagctgcctgtgatgaccacagaggaacccactaccccacccagaaacagtcctgggtcaacaactgaggcacccaccctgaccacacctgaaaatatcactaccgccgtgaaaacagtcctgcctcaggagtctactagtaacggactgatcaccagcacagtgactggaattctgggcagtctggggctgcgcaagcgatcaaggcgccaagtgaatactcgggctaccggcaaatgcaatccaaacctgcactactggaccgcacaggagcagcataacgccgctgggatcgcttggattccttacttcggaccaggcgcagaggggatctataccgaaggactgatgcataatcagaacgccctggtgtgtggcctgagacagctggcaaatgagacaactcaggccctgcagctgttcctgagagcaaccacagaactgaggacctatacaatcctgaaccggaaggccattgattttctgctgcgacgatggggcgggacctgcagaatcctgggaccagactgctgtattgagccccacgattggaccaagaacatcacagacaagatcaaccagatcattcatgatttcatcgacaacccactgcccaatcaggacaacgatgacaattggtggaccggatggcgacagtggattcccgcaggaattggaatcaccggaattattattgccattattgctctgctgtgtgtctgtaagctgctgtgttgataa

SEQ ID NO: 53 - protein, 676 aa, Sudan Ebolavirus
  misc_feature (68)..(68): Xaa can be any naturally occurring amino acid
  misc_feature (141)..(141): Xaa can be any naturally occurring amino acid
  misc_feature (170)..(170): Xaa can be any naturally occurring amino acid
  misc_feature (240)..(240): Xaa can be any naturally occurring amino acid
  misc_feature (348)..(348): Xaa can be any naturally occurring amino acid
MEGLSLLQLPRDKFRKSSFFVWVIILFQKAFSMPLGVVTNSTLEVTEIDQLVCKDHLASTDQLKSVGXNLEGSGVSTDIPSATKRWGFRSGVPPKVVSYEAGEWAENCYNLEIKKPDGSECLPPPPDGVRGFPRCRYVHKXQGTGPCPGDYAFHKDGAFFLYDRLASTVXYRGVNFAEGVIAFLILAKPKETFLQSPPIREAVNYTENTSSYYATSYLEYEIENFGAQHSTTLFKINNNXFVLLDRPHTPQFLFQLNDTIHLHQQLSNTTGKLIWTLDANINADIGEWAFWENKKNLSEQLRGEELSFETLSLNETEDDDATSSRTTKGRISDRATRKYSDLVPKDSXGMVSLHVPEGETTLPSQNSTEGRRVDVNTQETITETTATIIGTNGNNMQISTIGTGLSSSQILSSSPTMAPSPETQTSTTYTPKLPVMTTEEPTTPPRNSPGSTTEAPTLTTPENITTAVKTVLPQESTSNGLITSTVTGILGSLGLRKRSRRQVNTRATGKCNPNLHYWTAQEQHNAAGIAWIPYFGPGAEGIYTEGLMHNQNALVCGLRQLANETTQALQLFLRATTELRTYTILNRKAIDFLLRRWGGTCRILGPDCCIEPHDWTKNITDKINQIIHDFIDNPLPNQDNDDNWWTGWRQWIPAGIGITGIIIAIIALLCVCKLLC

SEQ ID NO: 54 - DNA, 2049 bp, Marburgvirus
  misc_feature (21)..(21): n is a, c, g, or t
atgaaaaccacttgtctgctnatctcactgattctgattcagggcgtcaaaacactgcccattctggaaattgcctctaacatccagccacagaacgtggactccgtctgttctgggaccctgcagaagacagaggatgtgcacctgatgggcttcaccctgagcgggcagaaggtcgcagactcacccctggaagccagcaaacgatgggcatttcgggccggagtgccccctaagaacgtcgagtacaccgaaggcgaggaagccaaaacatgctataatatctccgtgactgatcctagtggcaagtcactgctgctggacccacccaccaacattagggattaccctaagtgtaaaacaatccaccatattcagggccagaatccacacgctcaggggatcgcactgcatctgtggggagccttctttctgtacgacaggattgctagcaccacaatgtatcgcgggaaagtgttcaccgagggaaacatcgccgctatgattgtgaataagacagtccacaaaatgatcttttctcgccagggccaggggtaccgacatatgaacctgaccagtacaaataagtattggaccagctccaacggcactcagaccaatgacactgggtgcttcggaaccctgcaggagtacaacagtactaaaaatcagacctgtgctccatcaaagaaaccactgccactgcctaccgcacacccagaggtgaagctgacaagtacttcaaccgacgccacaaaactgaacactaccgaccccaatagtgacgatgaagatctgacaactagcggatccggctctggggagcaggaaccttataccacatccgatgcagccaccaagcagggcctgtctagtacaatgcctccaactccatctccccagcctagtactccccagcagggcgggaacaataccaaccattcccagggcgtggtcacagagccagggaagactaacactaccgcccagccctctatgccccctcacaatacaactaccatctccaccaacaatacatctaaacataacctgagcacaccttccgtgccaatccagaacgctactaactacaacactcagtctaccgcacccgagaatgaacagacttctgcccctagtaagacaactctgctgcccaccgagaaccctaccacagccaagtcaacaaatagcactaaatcccctactaccacagtgccaaacactaccaataagtacagtacctcaccaagccccacccctaactccacagcacagcacctggtctatttccggagaaaaagaaatatcctgtggagggagggcgacatgttcccttttctggatgggctgatcaacgctccaattgacttcgatccagtgcccaatacaaagactatctttgacgaatcaagctcctctggcgcctctgctgaggaagatcagcacgcctcacccaacattagcctgacactgtcctactttcctaaagtgaacgagaatactgcccatagcggggagaacgaaaatgactgcgatgctgagctgcggatctggagcgtccaggaagacgatctggctgcaggactgtcctggatcccattctttggacccggcattgagggactgtataccgccggcctgattaagaaccagaacaacctggtgtgcagactgaggcgcctggccaatcagaccgctaaatcactggaactgctgctgcgggtcacaactgaggaaagaacattcagcctgatcaaccgacatgctattgactttctgctggcacgctggggaggcacctgcaaggtgctgggaccagactgctgtatcggcattgaggatctgtctcgcaatatcagtgaacagatcgaccagattaagaaagatgagcagaaggaaggaaccggatggggactgggcggcaagtggtggaccagcgattggggcgtgctgacaaacctgggaatcctgctgctgctgtccatcgccgtcctgattgctctgtcctgtatttgtcggattttcactaagtatattgggtgataa

SEQ ID NO: 55 - protein, 681 aa, Marburgvirus
  misc_feature (7)..(7): Xaa can be any naturally occurring amino acid
  misc_feature (200)..(200): Xaa can be any naturally occurring amino acid
MKTTCLXISLILIQGVKTLPILEIASNIQPQNVDSVCSGTLQKTEDVHLMGFTLSGQKVADSPLEASKRWAFRAGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTXSNGTQTNDTGCFGTLQEYNSTKNQTCAPSKKPLPLPTAHPEVKLTSTSTDATKLNTTDPNSDDEDLTTSGSGSGEQEPYTTSDAATKQGLSSTMPPTPSPQPSTPQQGGNNTNHSQGVVTEPGKTNTTAQPSMPPHNTTTISTNNTSKHNLSTPSVPIQNATNYNTQSTAPENEQTSAPSKTTLLPTENPTTAKSTNSTKSPTTTVPNTTNKYSTSPSPTPNSTAQHLVYFRRKRNILWREGDMFPFLDGLINAPIDFDPVPNTKTIFDESSSSGASAEEDQHASPNISLTLSYFPKVNENTAHSGENENDCDAELRIWSVQEDDLAAGLSWIPFFGPGIEGLYTAGLIKNQNNLVCRLRRLANQTAKSLELLLRVTTEERTFSLINRHAIDFLLARWGGTCKVLGPDCCIGIEDLSRNISEQIDQIKKDEQKEGTGWGLGGKWWTSDWGVLTNLGILLLLSIAVLIALSCICRIFTKYIG

SEQ ID NO: 56 - DNA, 2999 bp, Artificial sequence
  pVAX1 backbone sequence
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 57 - DNA, 1221 bp, Artificial sequence
  NC99_60mer_pVax
ggatccgccaccatggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcgcccctctgcagctgggaaactgctccgtggcaggatggattctgggcaatccagagtgtgagctgctgatctctaaggagtcctggtcttacatcgtggagaccccaaaccccgagaatggcacatgctttcccggctacttcgccgactatgaggagctgagggagcagctgagctccgtgtctagcttcgagagatttgagatcttccctaaggagtcctcttggccaaaccacaccgtgacaggcgtgagcgcctcctgttctcacaacggcaagagctccttttataggaatctgctgtggctgaccggcaagaacggcctgtaccctaatctgagcaagtcctatgtgaacaataaggagaaggaggtgctggtgctgtggggcgtgcaccaccctcccaacatcggcaatcagagggccctgtaccacaccgagaacgcctacgtgagcgtggtgtctagccactacagcaggagattcacacccgagatcgccaagaggcctaaggtgcgcgaccaggagggacggatcaattactattggaccctgctggagccaggcgatacaatcatctttgaggccaacggcaatctgatcgccccctggtatgccttcgccctgtcccgcggctgataactcgag

SEQ ID NO: 58 - protein, 399 aa, Artificial sequence
  NC99_60mer_pVax
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRG

SEQ ID NO: 59 - DNA, 1230 bp, Artificial sequence
  NC99_g6_60mer_pVax
ggatccgccaccatggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcgcccctctgcagctgggaaactgcagcgtggcaggatggattctgggcaatccagagtgtgagctgctgatctccaaggagtcctggtcttacatcgtggagaccccaaaccccgagaatggcacatgctttcccggcaacttctctgactatgaggagctgagggagcagctgagctccgtgtctagcttcgagagatttgagatcttccctaaggagtcctcttggccaaatcacaccgtgacaggcgtgagcgcctcctgttctcacaacggcaagagctccttttacaggaatctgctgtggctgaccggcaagaacggcctgtaccctaatctgagcaagtcctataacaatacaaaggagaaggaggtgctggtgctgtggggcgtgcaccaccctcccaacatcggcaatcagagggccctgtaccacaccgagaacgcctacgtgagcgtggtgtctagccactactctaggagattcacacccaacatcagcaagaggcctaaggtgcgcgaccaggagggacggatcaattactattggaccctgctggagccaggcgatacaatcatctttgaggccaacggcaatctgatcgccccctggtatgccttcgccctgtctcgcggcaacggcagctgataactcgag

SEQ ID NO: 60 - protein, 402 aa, Artificial sequence
  NC99_g6_60mer_pVax
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGAPLQLGNCSVAGWILGNPECELLISKESWSYIVETPNPENGTCFPGNFSDYEELREQLSSVSSFERFEIFPKESSWPNHTVTGVSASCSHNGKSSFYRNLLWLTGKNGLYPNLSKSYNNTKEKEVLVLWGVHHPPNIGNQRALYHTENAYVSVVSSHYSRRFTPNISKRPKVRDQEGRINYYWTLLEPGDTIIFEANGNLIAPWYAFALSRGNGS

SEQ ID NO: 61 - DNA, 1227 bp, Artificial sequence
  CA09(175L)_Ferritin_pVax
atggactggacttggattctgtttctggtcgccgctgccactcgcgtgcattctgccccactgcacctgggcaagtgcaacatcgccggctggattctgggcaatcccgagtgtgagagcctgtccaccgccagctcctggagctacatcgtggagaccccttctagcgacaacggcacatgctttccaggcgacttcatcgattatgaggagctgagggagcagctgtcctctgtgagctccttcgagagatttgagatcttccccaagacctctagctggcctaaccacgattccaataagggagtgacagcagcatgtcctcacgcaggcgccaagagcttttacaagaacctgatctggctggtgaagaagggcaattcctacccaaagctgtctaagagctatatcaacgacaagggcaaggaggtgctggtgctgtggggcatccaccacccatccacctctgccgaccagcagtctctgtaccagaatgccgatacatacgtgttcgtgggctcctctcggtactccaagaagttcaagccagagatcgccatcaggcccaaggtgagagaccaggagggccgcatgaattactattggacactggtggagcccggcgataagatcacctttgaggccacaggcaacctggtggtgcctcggtatgccttcgccatggagcgcaatgcaagcggggaaagccaggtgcgacagcagttctccaaagacatcgaaaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcaggaagc

SEQ ID NO: 62 - protein, 409 aa, Artificial sequence
  CA09(175L)_Ferritin_pVax
MDWTWILFLVAAATRVHSAPLHLGKCNIAGWILGNPECESLSTASSWSYIVETPSSDNGTCFPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 63 - DNA, 2112 bp, Artificial sequence
  H1_CA04/09_FL_HA_3BVE_pVAX
atggactggacttggattctgttcctggtcgccgccgcaacccgcgtgcattctatgaaggctattctggtcgtgctgctgtatactttcgccaccgccaacgccgacacactgtgcatcggctaccacgccaacaattctaccgacacagtggataccgtgctggagaagaatgtgaccgtgacacacagcgtgaacctgctggaggataagcacaatggcaagctgtgcaagctgaggggagtggcaccactgcacctgggcaagtgcaacatcgccggctggattctgggcaatcccgagtgtgagtccctgtctacagccagctcctggtcctacatcgtggagacaccctctagcgacaacggcacatgctaccctggcgactttatcgattatgaggagctgcgggagcagctgagcagcgtgagcagcttcgagaggttcgagatcttccccaagacctctagctggcctaaccacgatagcaataagggagtgacagcagcatgtccacacgcaggcgccaagagcttctataagaacctgatctggctggtgaagaagggcaattcctaccctaagctgagcaagtcctatatcaacgacaagggcaaggaggtgctggtgctgtggggcatccaccacccatctaccagcgccgaccagcagtccctgtaccagaatgccgatacatacgtgttcgtgggctcctctcggtactctaagaagttcaagccagagatcgccatcaggccaaaggtgagggaccaggagggacgcatgaactactattggaccctggtggagcccggcgataagatcacctttgaggccacaggcaacctggtggtgcctagatatgccttcgccatggagagaaatgccggctccggcatcatcatctctgacacccctgtgcacgattgcaacaccacatgtcagaccccaaagggcgccatcaacacatccctgccttttcagaatatccacccaatcacaatcggcaagtgccctaagtacgtgaagagcaccaagctgaggctggcaacaggcctgcgcaatatcccatctatccagagcaggggcctgtttggagcaatcgcaggcttcatcgagggaggatggaccggaatggtggacggctggtacggctatcaccaccagaacgagcagggcagcggatatgcagcagacctgaagtccacccagaatgccatcgatgagatcacaaacaaggtcaattccgtgatcgagaagatgaacacccagtttacagccgtgggcaaggagttcaatcacctggagaagagaatcgagaacctgaataagaaggtggacgatggcttcctggacatctggacctacaacgccgagctgctggtgctgctggagaatgagaggacactggactaccacgattccaacgtgaagaatctgtatgagaaggtgagatctcagctgaagaacaatgccaaggagatcggcaacggctgcttcgagttttaccacaagtgcgacaacacctgtatggagagcgtgaagaatggcacatacgattatcctaagtattccgaggaggccaagctgaaccgcgaggagatcgactctggcggcgatatcatcaagctgctgaacgagcaagtgaataaggagatgcagagctccaatctgtacatgtctatgtctagctggtgttatacccacagcctggacggagcaggcctgttcctgtttgatcacgccgccgaggagtacgagcacgccaagaagctgatcatctttctgaacgagaacaatgtgccagtgcagctgacctccatctctgcccccgagcacaagtttgagggcctgacacagatcttccagaaggcctacgagcacgagcagcacatcagcgagtccatcaacaatatcgtggaccacgccatcaagagcaaggatcacgccaccttcaactttctgcagtggtacgtggccgagcagcacgaggaggaggtgctgttcaaggacatcctggataagatcgagctgatcggcaacgagaatcacgggctgtacctggcagaccagtatgtcaagggcatcgcaaagtcacggaagagcgggagc

SEQ ID NO: 64 - protein, 704 aa, Artificial sequence
  H1_CA04/09_FL_HA_3BVE_pVAX
MDWTWILFLVAAATRVHSMKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 65 - protein, 327 aa, Influenza A virus
FTATYADTICIGYHANNSTDTVDTVLEKNVTVTHSVNLLEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIH

SEQ ID NO: 66 - protein, 566 aa, Influenza A virus
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 67 - protein, 566 aa, Influenza A virus
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

(19) United States
(12) Patent Application Publication
(10) Pub. No.: US 2022/0047695 A1
(43) Pub. Date: Feb. 17, 2022

(54) COMPOSITIONS COMPRISING SELF-ASSEMBLING VACCINES AND METHODS OF USING THE SAME

(71) Applicant: The Wistar Institute of Anatomy and Biology, Philadelphia, PA (US)

(72) Inventors: Ziyang Xu, Philadelphia, PA (US); Daniel W. Kulp, Moorestown, NJ (US); David B. Weiner, Merion, PA (US)

(21) Appl. No.: 17/417,096

(22) PCT Filed: Dec. 23, 2019
(86) PCT No.: PCT/US2019/068444

§ 371 Date: Jun. 21, 2021

Related U.S. Application Data
(60) Provisional application No. 62/784,318, filed on Dec. 21, 2018.

(51) Int. Cl. A61K 39/21; C12N 7/00

(52) U.S. Cl. CPC A61K 39/21; A61K 2039/53; C12Y 205/01078; C12N 7/00

(57) ABSTRACT

Disclosed are compositions comprising an expressible nucleic acid sequence comprising a first nucleic acid sequence comprising a leader sequence or a pharmaceutically acceptable salt thereof; and a second nucleic acid sequence comprising a sequence that encodes a self-assembling polypeptide or a pharmaceutically acceptable salt thereof. In some embodiments, the expressible nucleic acid sequence further comprises a nucleic acid sequence encoding at least one viral antigen or a pharmaceutically acceptable salt thereof. In some embodiments, the expressible nucleic acid sequence further comprises at least one nucleic acid sequence encoding a linker. Also disclosed are pharmaceutical compositions comprising these compositions and methods of using the disclosed compositions.

### CROSS-REFERENCE TO RELATED APPLICATIONS

This application is a national stage application filed under 35 U.S.C. § 371 of International Application No. PCT/US2019/068444, filed Dec. 23, 2019, which claims benefit of U.S. Provisional Application No. 62/784,318, filed Dec. 21, 2018, and the content of each of the aforementioned applications is hereby incorporated by reference in its entirety.

### STATEMENT REGARDING FEDERALLY SPONSORED RESEARCH

The present disclosure was made with government support under U19 A1109646-04 awarded by the National Institutes of Health. The government has certain rights in the present disclosure.

### SEQUENCE LISTING

This application is submitted with a Sequence Listing text file in ASCII format, which serves as both the computer readable form (CFR) and the paper copy as required under 37 C.F.R. § 1.821. Said Sequence Listing text file is: entitled “37925_0004P1_12_23_SL.txt”, 148,135 bytes in size, created on Jun. 19, 2021, and incorporated by reference in its entirety.

### BACKGROUND

Vaccination, a process in which antigenic materials are introduced in a host to elicit specific adaptive immunity, has proven to be an extremely efficacious prophylactic measure against various infectious diseases. While the vaccines currently approved by the FDA have remarkable public health value, significant improvements can still be made. For example, while the current quadrivalent inactivated influenza vaccines are efficient at inducing autologous neutralizing antibodies, they cannot efficiently induce humoral responses with broad coverage such that annual immunization with stocks that correspond the strains predicted to circulate is necessary. In the HIV space, induction of broadly neutralizing antibodies through active immunization has been identified as an extremely important approach to reduce global incidences of HIV-1 infections. While significant advances have been made in strategies that may result in elicitation of such antibodies in humans, with the use of either germline-targeting immunogens¹⁻⁴ or stabilized native-like trimers⁵⁻⁷ relevant humoral responses are generated typically after multiple immunizations that can span over an entire year, and often result in induction of inconsistent responses across animals studied. Further, native-like trimers induce low and short-lived antibody titers in rhesus macaques, an important pre-clinical animal models for HIV vaccine development. An approach that results in more rapid induction of relevant humoral immunity is of critical need to translate promising vaccine candidates into the clinics. In the past-decade, advances in material engineering has created opportunities for the explorations of novel vaccine concepts such as nanoparticle vaccines^{8,9}. Nanoparticles, ranging from 20-200 nm in diameter, present antigens in a repetitive fashion and can robustly stimulate long-lasting humoral responses¹⁰⁻¹². However, large-scale translation of these nanoparticle vaccines into the clinical space remains challenging due to difficulties in the synthesis and purification of these nanoparticles¹³. Physical/electrostatic adsorption techniques used to conjugate nanomaterials to antigens may lack substrate specificities and gather contaminants; chemical conjugations involving functionalization of the protein amine/thiol groups can potentially alter the antigenic profiles; protein-capsid based virus-like particle (VLP) vaccines produced from cell lines are difficult to purify, frequently requiring both disassembly and reassembly to achieve proper folding^{14,15} Technologies that would allow de novo nanoparticle assemblies in the hosts can potentially surpass the synthesis and purification steps, facilitate rapid translation of promising vaccine candidates into the clinic and therefore be of major advantage. In addition, current protein nanoparticles cannot efficiently induce CD8+ T-cell responses¹⁶, severely limiting their utility in clearing viral pathogens that require Cytotoxic T Lymphocyte (CTL)− such as reduction of HIV-1 reservoir in humans. In this patent, we have demonstrated a novel way to produce in vivo potent nanoparticle vaccines using advanced synthetic nucleic acid electroporation technology that bypass the need for cumbersome in vitro assembly/purification steps. In addition, the nucleic-acid launched nanoparticles quickly induce robust and durable humoral responses, and significantly stronger CTL responses in comparison to protein/adjuvant-based nanoparticle vaccines. We have also demonstrated that the nucleic-acid launched nanoparticle platform does not depend on extensive opsonization by components of the innate immune system, unlike protein/adjuvant-based nanoparticles, showing that engineering/design of nanoparticle vaccines in the nucleic acid platform can be significantly simpler and faster.

### SUMMARY OF EMBODIMENTS

There are significant limitations in administering therapeutically effective amounts of protein vaccines to subjects, including ensuring that appropriate levels of the vaccine become exposed to antigen presenting cells and ensuring that the magnitude of any immune response is sufficient after administration of a single bolus dose. Furthermore, protein vaccines are difficult to store for relatively long periods of time because of protein instability issues. DNA vaccination is an alternative vaccination technique but suffers from delivery difficulties. Encapsulation and delivery of DNA vaccines have been used in the context of gene therapy but are incredibly expensive processes that require extended periods of time for preparation and isolation of particles. Encapsulating the DNA vaccines with particle still has limitations with storage of the complex molecules.

To address these limitations and address the limitations associated with manufacturing nanoparticles, the present disclosure relates to nucleic acid sequences that encode self-assembling nanoparticles and peptide antigens and compositions comprising the same. In some embodiments, the disclosure relates to compositions comprising an expressible nucleic acid sequence comprising a first nucleic acid sequence comprising a leader sequence or a pharmaceutically acceptable salt thereof; and a second nucleic acid sequence comprising a sequence that encodes a self-assembling polypeptide or a pharmaceutically acceptable salt thereof. In some embodiments, the expressible nucleic acid sequence further comprises a third nucleic acid sequence encoding a viral antigen. When the nucleic acid sequence is adminstered to a subject in the context of a method of treatment or prevention of the viral infection, antigen presenting cells can be transduced or transfected with the antigens encoded by the expressible nucleic acid sequence.

Disclosed are compositions comprising an expressible nucleic acid sequence comprising a first nucleic acid sequence comprising at least about 70% sequence identity to SEQ ID NO:1 or encoding a polypeptide comprising at least about 70% sequence identity to SEQ ID NO:6, or a pharmaceutically acceptable salt thereof; and a second nucleotide sequence comprising at least about 70% sequence identity to SEQ ID NO:2 or encoding a polypeptide comprising at least about 70% sequence identity to SEQ ID NO:7, or a pharmaceutically acceptable salt thereof. Also disclosed are compositions comprising an expressible nucleic acid sequence comprising a nucleic acid sequence comprising at least about 70% sequence identity to SEQ ID NO:1 or encoding a polypeptide comprising at least about 70% sequence identity to SEQ ID NO:6, or a pharmaceutically acceptable salt thereof and a nucleotide sequence encoding a self-assembling polypeptide.

In some embodiments, the expressible nucleic acid sequence further comprises a nucleic acid sequence encoding at least one viral antigen or a pharmaceutically acceptable salt thereof. In some embodiments, the viral antigen is an antigen from a Retroviridae or Flavivirus or Nipah Virus or Influenza Virus or any virus disclosed in Table 1. In some embodiments, the viral antigen is an antigen from human immunodeficiency virus-1 (HIV-1). In some embodiments, the viral antigen comprises at least about 70% sequence identity to SEQ ID NO: 9 or a pharmaceutically acceptable salt thereof. In some embodiments, the viral antigen is an antigen from West Nile virus. In some embodiments, the viral antigen is an antigen from human papillomavirus. In some embodiments, the viral antigen is an antigen from respiratory syncytial virus. In some embodiments, the viral antigen is an antigen from filovirus. In some embodiments, the viral antigen is an antigen from Zaire ebolavirus. In some embodiments, the viral antigen is an antigen from Sudan ebolavirus. In some embodiments, the viral antigen is an antigen from marburgvirus. In some embodiments, the viral antigen is an antigen from influenza virus.

In some embodiments, the expressible nucleic acid sequence further comprises at least one nucleic acid sequence encoding a linker. In some embodiments, the at least one nucleic acid sequence encoding a linker comprises at least about 70% sequence identity to SEQ ID NO:3 or a pharmaceutically acceptable salt thereof. In some embodiments, the expressible nucleic acid sequence is operably linked to one or a plurality of regulatory sequences.

In some embodiments, the expressible nucleic acid sequence is comprised in a nucleic acid molecule. In some embodiments, the nucleic acid molecule is a plasmid. In some embodiments, the plasmid comprises an expressible nucleic acid sequence comprising at least about 70% sequence identity to SEQ ID NO:5, SEQ ID NO:11, SEQ ID NO:20, SEQ ID NO:24, SEQ ID NO:28, SEQ ID NO:33, SEQ ID NO:34, SEQ ID NO:37, SEQ ID NO:57, SEQ ID NO:59, SEQ ID NO:61 or SEQ ID NO:63, or a pharmaceutically acceptable salt thereof. In some embodiments, the plasmid comprises an expressible nucleic acid sequence encoding a polypeptide comprising at least about 70% sequence identity to SEQ ID NO:10, SEQ ID NO:12, SEQ ID NO:21, SEQ ID NO:25, SEQ ID NO:30, SEQ ID NO:35, SEQ ID NO:36, SEQ ID NO:38, SEQ ID NO:58, SEQ ID NO:60, SEQ ID NO:62, SEQ ID NO:64, SEQ ID NO:65, SEQ ID NO:66 or SEQ ID NO:67, or a pharmaceutically acceptable salt thereof.

The disclosure also relates to pharmaceutical compositions comprising any one or more of the disclosed compositions and a pharmaceutically acceptable carrier. In some embodiments, the pharmaceutical composition comprises from about 1 to about 100 micrograms of the disclosed composition. In some embodiments, the pharmaceutical composition comprises from about 1 to about 20 micrograms of the disclosed composition.

Disclosed are methods of vaccinating a subject comprising administering a therapeutically effective amount of any of the disclosed pharmaceutical compositions to the subject. The disclosure further relates to methods of inducing an immune response in a subject comprising administering to the subject any of the disclosed pharmaceutical compositions. Also disclosed are methods of neutralizing one or a plurality of viruses in a subject comprising administering to the subject any of the disclosed pharmaceutical compositions.

Disclosed are methods of stimulating a therapeutically effective antigen-specific immune response against a virus in a mammal infected with the virus comprising administering any of the disclosed pharmaceutical compositions. Also disclosed are methods of inducing expression of a self-assembling vaccine in a subject comprising administering any of the disclosed pharmaceutical compositions.

In some embodiments, the administering in any of the methods disclosed herein is accomplished by oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or combinations thereof. In some embodiments, the therapeutically effective amount is from about 20 to about 2000 micrograms of the expressible nucleic acid sequence. In some embodiments, the therapeutically effective amount is from about 0.3 micrograms of composition per kilogram of subject to about 30 micrograms per kilogram of subject. In some embodiments, any of the disclosed methods is free of activating any mannose-binding lectin or complement process. In some embodiments, the subject being administered is a human.

Disclosed are vaccines comprising a first amino acid sequence comprising at least about 70% sequence identity to SEQ ID NO:7; and/or a second amino acid sequence comprising at least about 70% sequence identity to SEQ ID NO:9. Disclosed are vaccines comprising a first amino acid sequence comprising at least about 70% sequence identity to SEQ ID NO:7; and/or a second amino acid sequence comprising at least about 70% sequence identity to any viral antigen disclosed herein. Disclosed are vaccines comprising a first amino acid sequence comprising at least about 70% sequence identity to any self-assembling polypeptide disclosed herein; and/or a second amino acid sequence comprising at least about 70% sequence identity to SEQ ID NO:9. In some embodiments, the vaccine comprises a leader sequence comprising at least about 70% sequence identity to SEQ ID NO:6. In some embodiments, the vaccine disclosed herein further comprises a linker fusing the first and second amino acid sequences. In some embodiments, the linker is an amino acid sequence comprising at least about 70% sequence identity to SEQ ID NO:8.

Also disclosed is a DNA vaccine comprising an expressible nucleic acid sequence encoding a polypeptide comprising at least about 70% sequence identity to SEQ ID NO:10, SEQ ID NO:21, SEQ ID NO:25, SEQ ID NO:30, SEQ ID NO:35, SEQ ID NO:36, SEQ ID NO:38, SEQ ID NO:58, SEQ ID NO:60, SEQ ID NO:62, SEQ ID NO:64, SEQ ID NO:65, SEQ ID NO:66 or SEQ ID NO:67, or a pharmaceutically acceptable salt thereof. In some embodiments, the expressible nucleic acid sequence comprises at least about 70% sequence identity to SEQ ID NO:5, SEQ ID NO:20, SEQ ID NO:24, SEQ ID NO:28, SEQ ID NO:33, SEQ ID NO:34, SEQ ID NO:37, SEQ ID NO:57, SEQ ID NO:59, SEQ ID NO:61 or SEQ ID NO:63, or a pharmaceutically acceptable salt thereof. In some embodiments, the DNA vaccine further comprises a pharmaceutically acceptable excipient. In some embodiments, the pharmaceutically acceptable excipient is an adjuvant.

In another aspect, the present disclosure relates to a composition comprising one or a plurality of expressible nucleic acid sequences, the plurality of expressible nucleic acid sequences comprising a first nucleic acid sequence encoding a self-assembling polypeptide and a second nucleic acid sequence encoding a viral antigen and a third nucleic acid sequence encoding a leader peptide. In some embodiments, the first nucleic acid sequence encoding a self-assembling polypeptide comprises at least about 70% sequence identity to SEQ ID NO:2, SEQ ID NO:13, SEQ ID NO:14 or SEQ ID NO:15. In some embodiments, the self-assembling polypeptide comprises at least about 70% sequence identity to SEQ ID NO:7, SEQ ID NO:23, SEQ ID NO:31 or SEQ ID NO:26. In some embodiments, the viral antigen encoded by the second nucleic acid sequence is an antigen from a retroviridae, flavivirus, Nipah Virus, West Nile virus, human papillomavirus, respiratory syncytial virus, filovirus, zaire ebolavirus, sudan ebolavirus, marburgvirus or influenza virus. In some embodiments, the second nucleic acid sequence encoding a viral antigen comprises at least about 70% sequence identity to SEQ ID NO:41, SEQ ID NO:42, SEQ ID NO:43, SEQ ID NO:44, SEQ ID NO:46, SEQ ID NO:48, SEQ ID NO:50, SEQ ID NO:52, or SEQ ID NO:54. In some embodiments, the viral antigen comprises at least about 70% sequence identity to SEQ ID NO:45, SEQ ID NO:47, SEQ ID NO:49, SEQ ID NO:51, SEQ ID NO:53, or SEQ ID NO:55. In some embodiments, the third nucleic acid sequence encoding a leader peptide comprises at least about 70% sequence identity to SEQ ID NO:1 or SEQ ID NO:39. In some embodiments, the leader peptide comprises at least about 70% sequence identity to SEQ ID NO:6 or SEQ ID NO:40.

In some embodiments, the expressible nucleic acid sequence further comprises at least one linker between the first and second nucleic acid sequences, the second and third nucleic acid sequences, or the first and the third nucleic acid sequences. In some embodiments, the expressible nucleic acid sequence further comprises at least one linker between the first and second nucleic acid sequences and the second and third nucleic acid sequences. In some embodiments, the expressible nucleic acid sequence comprises, in 5′ to 3′ direction, the third nucleic acid sequence, the first nucleic acid sequence, and the second nucleic acid sequence, and at least one linker between each of the first and third nucleic acid sequences and the first and second nucleic acid sequences. In some embodiments, the at least one linker comprises at least about 70% sequence identity to SEQ ID NO:3, SEQ ID NO:16, SEQ ID NO:17, SEQ ID NO:19 or SEQ ID NO:29. In some embodiments, the at least one linker encodes a polypeptide comprising at least about 70% sequence identity to SEQ ID NO:8, SEQ ID NO:18, SEQ ID NO:22, SEQ ID NO:27 or SEQ ID NO:32.

In some embodiments, at least one of the plurality of expressible nucleic acid sequences comprises at least about 70% sequence identity to SEQ ID NO:5, SEQ ID NO:20, SEQ ID NO:24, SEQ ID NO:28, SEQ ID NO:33, SEQ ID NO:34, SEQ ID NO:37, SEQ ID NO:57, SEQ ID NO:59, SEQ ID NO:61 or SEQ ID NO:63. In some embodiments, at least one of the plurality of expressible nucleic acid sequences encodes a polypeptide comprises at least about 70% sequence identity to SEQ ID NO:10, SEQ ID NO:21, SEQ ID NO:25, SEQ ID NO:30, SEQ ID NO:35, SEQ ID NO:36, SEQ ID NO:38, SEQ ID NO:58, SEQ ID NO:60, SEQ ID NO:62, SEQ ID NO:64, SEQ ID NO:65, SEQ ID NO:66 or SEQ ID NO:67. In some embodiments, at least one of the plurality of expressible nucleic acid sequences is operably linked to at least one regulatory sequence.

A further aspect of the present disclosure relates to a cell comprising an expressible nucleic acid sequence comprising a first nucleic acid sequence encoding a self-assembling polypeptide and a second nucleic acid sequence encoding a viral antigen and a third nucleic acid sequence encoding a leader peptide. In some embodiments, the first nucleic acid sequence encoding a self-assembling polypeptide comprises at least about 70% sequence identity to SEQ ID NO:2, SEQ ID NO:13, SEQ ID NO:14 or SEQ ID NO:15. In some embodiments, the self-assembling polypeptide comprises at least about 70% sequence identity to SEQ ID NO:7, SEQ ID NO:23, SEQ ID NO:31 or SEQ ID NO:26. the viral antigen encoded by the second nucleic acid sequence is an antigen from a retroviridae, flavivirus, Nipah Virus, West Nile virus, human papillomavirus, respiratory syncytial virus, filovirus, zaire ebolavirus, sudan ebolavirus, marburgvirus or influenza virus. the second nucleic acid sequence encoding a viral antigen comprises at least about 70% sequence identity to SEQ ID NO:41, SEQ ID NO:42, SEQ ID NO:43, SEQ ID NO:44, SEQ ID NO:46, SEQ ID NO:48, SEQ ID NO:50, SEQ ID NO:52, or SEQ ID NO:54. In some embodiments, the viral antigen comprises at least about 70% sequence identity to SEQ ID NO:45, SEQ ID NO:47, SEQ ID NO:49, SEQ ID NO:51, SEQ ID NO:53, or SEQ ID NO:55. In some embodiments, the third nucleic acid sequence encoding a leader peptide comprises at least about 70% sequence identity to SEQ ID NO:1 or SEQ ID NO:39. In some embodiments, the leader peptide comprises at least about 70% sequence identity to SEQ ID NO:6 or SEQ ID NO:40.

In some embodiments, the expressible nucleic acid sequence further comprises at least one linker between the first and second nucleic acid sequences, the second and third nucleic acid sequences, or the first and the third nucleic acid sequences. In some embodiments, the expressible nucleic acid sequence further comprises at least one linker between the first and second nucleic acid sequences and the second and third nucleic acid sequences. In some embodiments, the expressible nucleic acid sequence comprises, in 5′ to 3′ direction, the third nucleic acid sequence, the first nucleic acid sequence, and the second nucleic acid sequence, and at least one linker between each of the first and third nucleic acid sequences and the first and second nucleic acid sequences. In some embodiments, the at least one linker comprises at least about 70% sequence identity to SEQ ID NO:3, SEQ ID NO:16, SEQ ID NO:17, SEQ ID NO:19 or SEQ ID NO:29. In some embodiments, the at least one linker encodes a polypeptide comprising at least about 70% sequence identity to SEQ ID NO:8, SEQ ID NO:18, SEQ ID NO:22, SEQ ID NO:27 or SEQ ID NO:32.

In some embodiments, the expressible nucleic acid sequence comprises at least about 70% sequence identity to SEQ ID NO:5, SEQ ID NO:20, SEQ ID NO:24, SEQ ID NO:28, SEQ ID NO:33, SEQ ID NO:34, SEQ ID NO:37, SEQ ID NO:57, SEQ ID NO:59, SEQ ID NO:61 or SEQ ID NO:63. In some embodiments, the expressible nucleic acid sequence encodes a polypeptide comprises at least about 70% sequence identity to SEQ ID NO:10, SEQ ID NO:21, SEQ ID NO:25, SEQ ID NO:30, SEQ ID NO:35, SEQ ID NO:36, SEQ ID NO:38, SEQ ID NO:58, SEQ ID NO:60, SEQ ID NO:62, SEQ ID NO:64, SEQ ID NO:65, SEQ ID NO:66 or SEQ ID NO:67.

In some embodiments, the expressible nucleic acid sequence comprised in the disclosed cell further comprises at least one regulatory sequence operably linked to the expressible nucleic acid sequence.

Also disclosed are pharmaceutical compositions comprising (i) any of the compositions disclosed herein or any of the cells disclosed herein and (ii) a pharmaceutically acceptable carrier. In some embodiments, the pharmaceutically acceptable carrier is an adjuvant. In some embodiments, the pharmaceutical composition comprises from about 1 to about 100 micrograms of the expressible nucleic acid sequence. In some embodiments, the pharmaceutical composition comprises from about 1 to about 20 micrograms of the expressible nucleic acid sequence.

A yet another aspect of the present disclosure relates to methods of vaccinating a subject against viral infection comprising administering a therapeutically effective amount of any of the pharmaceutical compositions disclosed herein. In some embodiments, the viral infection is an infection of retroviridae, flavivirus, Nipah Virus, West Nile virus, human papillomavirus, respiratory syncytial virus, filovirus, zaire ebolavirus, sudan ebolavirus, marburgvirus or influenza virus.

In another aspect, the present disclosure relates to methods of inducing an immune response to a viral antigen in a subject comprising administering a therapeutically effective amount of any of the pharmaceutical compositions disclosed herein. In some embodiments, the viral antigen is an antigen from a retroviridae, flavivirus, Nipah Virus, West Nile virus, human papillomavirus, respiratory syncytial virus, filovirus, zaire ebolavirus, sudan ebolavirus, marburgvirus or influenza virus. In some embodiments, the immune response is a viral antigen-specific immune response.

In some embodiments, the pharmaceutical composition is administered in any of the disclosed methods by oral administration, parenteral administration, sublingual administration, intradermal, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or combinations thereof. In some embodiments, the therapeutically effective amount used in any of the disclosed methods is from about 20 to about 2000 micrograms of the expressible nucleic acid sequence. In some embodiments, the therapeutically effective amount used in any of the disclosed methods is from about 0.3 micrograms of the expressible nucleic acid sequence per kilogram of subject to about 30 micrograms of the expressible nucleic acid sequence per kilogram of subject. In some embodiments, any of the disclosed methods is free of activating any mannose-binding lectin or complement process. In some embodiments, the subject is a human.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the subject matter as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A, 1B, and 1C show the platform for optimization for DNA-launching of self-assembling nanoparticle vaccines. FIG. 1A depicts a study showing IgE leader sequences support trafficking of target protein into the secretory network. FIG. 1B depicts a strategy to enhance DNA cassettes encoding an IgE leader sequence with tRNA codon optimization, mRNA secondary structure optimization, and amino acid usage utilization. FIG. 1C depicts DNA cassettes that encode self assembling nanoparticles in vivo. IgE-GLT1 comprises an N-terminal IgE leader sequence to drive trafficking of antigens to cellular secretory pathway followed by eOD-GT8. IgE-GLT1-NP comprises an N-terminal IgE leader sequence, followed by C-term of Lumazine synthase enzyme to drive for spontaneous self-assembly of these nanoparticle vaccines, followed by eOD-GT8 at the C-terminus of this construct.

FIGS. 2A, 2B, 2C, 2D, and 2E shows that IgE-GLT1 and IgE-GLT1-NP are expressed in vitro. Transfected 293T cells were stained with VRC01 and Goat-anti-human 488 and DAPI. Optimization to incorporate N-terminal IgE-leader sequence significantly boosts in vitro expression. A) GLT1-NP w/o IgE-LS; B) GLT1 w/o IgE-LS; C) IgE-GLT1-NP; D) IgE-GLT1; E) pVAX.

FIGS. 3A, 3B, and 3C show that multimerized IgE-GLT1-NP can be detected from Expi293F transfection supernatant. A) Transfection Supernatant-SDS PAGE; B) Transfection supernatant-Native PAGE; C) Size Exclusion Chromatography. To screen for multimerization of IgE-GLT1-NP encoded by the DNA plasmid, transfection of Expi293F cells were carried out and cell culture supernatant was collected. Native gel electrophoresis was used to analyze multimerization pattern of IgE-GLT1-NP (with VRC01 as the primary antibody). IgE-GLT1-NP produce bands that migrate less than IgE-GLT1, giving evidence of in vitro multimerization of this construct. Size exclusion chromatography of transfections supernatant confirms that IgE-GLT1-NP was secreted in the 60mer form as predicted by the retention time (which allows for estimation of the molecular weight).

FIGS. 4A, 4B, 4C, and 4D show an example of in vivo expression of antigens by Confocal Microscopy. A) schematic diagram of injection schedule. Immunocompetent balb/c mice were injected with 100 ug of DNA-encoded IgE-GTL1-NP and sacrificed 7 d.p.i.; B) Naïve mice; C) IgE-GLT1; D) IgE-GLT1-NP. Using confocal microscopy, in vivo production of IgE-GLT1-NP in mice skeletal muscles is seen.

FIG. 5 shows a native PAGE Western providing first evidence of in vivo assembly/multimerization of nanoparticle vaccines. Using Native Page gel to analyze the muscle extracts of mice immunized with either IgE-GLT1-NP or IgE-GLT1, both immunogens can be detected in vivo. In addition, the IgE-GLT1-NP produce bands that migrate less than IgE-GLT1, giving first evidence of in vivo multimerization of IgE-GLT1-NP.

FIGS. 6A, 6B, and 6C depict microscopy images of vaccine encoded by transfected cells. FIG. 6A depicts low magnification of transmission electron microscopy (TEM) image of muscle section from mice immunized with IgE-GTL1-NP depicts. FIG. 6B depicts high magnification of IgE-GLT1-NP immunized muscle section showing formation of gold-labelled nanoparticles in the vesicles. FIG. 6C depicts high magnification of naïve mice muscle section demonstrating low background with the staining process.

FIGS. 7A, 7B depict extensive nanoparticle multimerization does not occur in the ER in vivo. FIGS. 7A and 7B depict TEM images of muscle sections from mice immunized with DNA-encoded IgE-GLT1-NP demonstrating the cellular endoplasmic reticulum is not the site for assembly of nanoparticles in vivo.

FIGS. 8A and 8B show well-formed DNA-launched nanoparticles observed in secretory vesicles in vivo. FIGS. 8A and 8B depict TEM images of muscle sections from mice immunized with DNA-encoded IgE-GLT1-NP, showing well-formed VRC01-gold labelled IgE-GLT1-NP nanoparticles in the cellular vesicles (11 and 17 visible copies of VRC01 binding are observed).

FIG. 9 shows DNA-launched nanoparticle traffics more efficiently to draining lymph nodes corresponding monomeric antigen. Nanoparticle antigen gets taken up into draining LN for presentation by FDCs more effectively than monomeric antigens.

FIGS. 10A, 10B, and 10C show IgE-GLT1-NP induces sero-conversion as designed DNA encoded molecule 1 week post injection. A) 7 d.p.i.; B) 14 d.p.i.; C) 21 d.p.i. Using ELISA, it was shown that mice injected with DNA-encoded IgE-GLT1-NP seroconverted 1 week post single injection. In contrast, IgE-GLT1 vaccinated mice do not have appreciable titers until 3 weeks post injection.

FIGS. 11A and 11B show IgE-GLT1-NP induces more potent humoral responses than IgE-GLT1mer. Strong and durable humoral responses are seen even with a single injection. 1 shot or 2 shots of DNA-encoded IgE-GLT1-NP resulted in higher antibody titers than DNA-encoded IgE-GLT1 for 19 weeks post injection (1-2 log difference).

FIGS. 12A, 12B, and 12C show SynDNA-launched IgE-GLT1-NP induces stronger responses in mouse strains with different haplotypes. A) schematic diagram of injection schedule; B) Balb/c responses; C) C57BL/6 responses. The same pattern holds across mice with different haplotypes (balb/C and C57BL/6). No seroconversion was seen in C57BL/6 mice with 2 shots of DNA-encoded IgE-GLT1 whereas high titers were observed for IgE-GLT1-NP.

FIGS. 13A, 13B, and 13C show extremely low dose of synDNA-encoded IgE-GLT1-NP induces robust humoral responses. A dose-dependent increase in humoral response to GT8 was observed only for IgE-GLT1. A) schematic diagram of injection schedule; B) Endpoint titers with monomers; C) Endpoint titers with nanoparticles.

FIGS. 14A and 14B show a competition binding ELISA assay and demonstrates that sera from mice immunized twice with DNA-encoded IgE-GLT1-NP can potently outcompete potent binding of VRC01 to monomeric IgE-GLT1, demonstrating proper in vivo folding of nanoparticles to expose the CD4 binding site on the immunogen for recognition. Both panels demonstrate that DNA-encoded IgE-GLT1-NP induces stronger epitope-specific responses than DNA-encoded IgE-GLT1.DNA-encoded IgE-GLT1-NP immunization was found to be able to induce functional responses in balb/C mice. Sera from mice immunized with 100 ug of either DNA-encoded IgE-GLT1 or DNA-encoded IgE-GLT1-NP 4 weeks post 2nd dose was used in a competition ELISA assay. Mice antibodies produced in IgE-GLT1-NP mice were found to be able to outcompete VRC01 binding to GT8 more efficiently than IgE-GLT1 immunized mice by 3 fold.

FIGS. 15A and 15B show improving cellular immunity is a unique challenge. Prior Nanoparticles have had little impact on cellular immunity. The engineered IgE-GTL1-NP induces more robust cellular responses than IgE-GLT1. A) schematic diagram of injection schedule; B) Elispot assay. IgE-GLT1-NP was found to induce stronger cellular responses than IgE-GLT1 as determined by IFN-g ELIspot assays. 25 ug of DNA-encoded IgE-GLT1-NP can induce 500× stronger IFNg responses than 25 ug of DNA-encoded IgE-GLT1

FIG. 16 shows DNA-launched nanoparticles induce more potent CD4-memory responses than corresponding monomers. Using Flow-base intracellular cytokine staining assay to look at memory CD4-T-cells responses (CD3+ C4+ CD44+ CD62L− subsets), IgE-GLT1-NP immunized mice were found to have stronger CD4 responses than IgE-GLT1 immunized mice. Remarkably, the LS domain provides more robust CD4-T-cell help than the GT8 domain.

FIG. 17 shows BALB/c CD8 memory T-cell responses to GT8 domain. DNA-encoded IgE-GLT1-NP can induce stronger CD8-T-cell responses to the antigenic domain in balb/c mice when we examine cytokine activation (IL-2, TNFa and IFNg) and degranulation marker CD107a than DNA-encoded IgE-GLT1. The cells are CD8+ CD44+ CD62L−.

FIG. 18 shows designed DNA launched nanoparticle assembly enhances CD8 response in BALB/c mice. DNA-launched nanoparticles induce 10× stronger CD8 responses than corresponding monomer.

FIG. 19 shows DNA-launched nanoparticles increase formation of CD8 memory T-cells.

FIG. 20 is a schematic diagram of the MBL complement pathway for enhanced antigen opsonization and uptake.

FIGS. 21A, and 21B show even DNA-launched nanoparticle can activate MBL in vivo, immunogenicity of DNA-based nanoparticles is independent of MBL, unlike proteins. A) Immunogenicity of protein nanoparticles is dependent on lectin-complement pathway. As responses induced in MBL and complement receptor knockouts mice are significantly lower than the wildtype controls. B) DNA-launched nanoparticles, however, can still robustly induce responses in these transgenic animals, showing an Independence of the lectin-complement pathway (even though DNA-launched nanoparticles are fully capable of binding to MBL as shown previously).

FIGS. 22A and 22B show In vitro produced IgE-GLT1-NP binds to Mannose-Binding Lectin with higher affinity than IgE-GLT1. A) VRC01 binding of in vitro purified antigens; B) MBL binding of in vitro purified antigens. Previous studies determined that activation of MBL-complement pathway is important for immunogenicity of protein-based eOD-GT8-60mer. Consistent with their reports, IgE-GLT1-NP expressed from the currently disclosed DNA-cassette can bind to MBL more strongly than monomeric IgE-GLT1. As a control, protein-based IgE-GLT1-NP and IgE-GLT1 bind to VRC01 equally strongly.

FIGS. 23A and 23B show DNA-launched in vivo expressed IgE-GLT1-NP also binds to MBL more strongly than IgE-GLT1. A) VRC01 binding of in vitro purified antigens; B) MBL binding of in vitro purified antigens. Similarly, the in vivo produced IgE-GLT1-NP in the muscle extract (7 d.p.i) was found to bind to MBL more strongly than in vivo produced IgE-GLT1. As a control, IgE-GLT1 was found to bind to VRC01 equally strongly.

FIGS. 24A, 24B, 24C, 24D show DNA-launched IgE-GLT1-NP binds to endogenous MBL in vivo with anti-MBL IHC. A) schematic diagram of experimental design; B) Naïve mice; C) IgE-GLT1; D) IgE-GLT1-NP. IHC-assay was used to determine in vivo labelling of muscles by MBL upon DNA-encoded IgE-GLT1-NP immunization. As compared to Naïve mice, IgE-GLT1-NP immunized mice demonstate strong labelling of muscles (at the injected site) by MBL, demonstrating DNA-launched nanoparticles are fully capable of activating the MBL-pathway in vivo.

FIGS. 25A, 25B, 25C, 25D, 25E compare immunogenicity induced by either protein-based nanoparticle or Nucleic acid launched nanoparticles in C57BL/6 mice. A) schematic diagram of vaccination schedule; B) protein-based and nucleic acid launched nanoparticles induce similar humoral responses in mice; C) nucleic acid launched nanoparticles induce 2.2-fold higher cellular responses than protein-based nanoparticles by IFNγ ELIspot assay; D) intracellular cytokine staining shows that protein and DNA-launched nanoparticles induce similar CD4 memory T-cell responses in mice; E) shows only DNA-launched nanoparticles, but not protein-based nanoparticles, can induce CD8 T-cell responses in mice.

FIG. 26 shows the design and evaluation of new DLnano GT8-vaccines with alternative scaffolds to determine generalizability of the system. a. nsEM image of SEC-purified fraction of in vitro produced 3BVE-GT8 nanoparticles. b. nsEM image of SEC-purified fraction of in vitro produced PfV-GT8 nanoparticles. c. In vivo expression of DLnano_3BVE_GT8 and DLnano_PfV_GT8 in transfected mouse muscles as determined by immunofluorescence; VRC01 labelling is shown in light gray and nuclei labelling shown in dark gray. d. Reducing SDS PAGE western analysis to determine in vivo expression of DLnano_3BVE_GT8 and DLnano_PfV_GT8 four d.p.i in muscle homogenates with VRC01 (in light gray); GAPDH (in dark gray) is used as the loading control. e. Humoral responses in BALB/c mice immunized with two 25 μg doses of DLmono_GT8, DLnano_3BVE_GT8, DLnano_LS_GT8 and DLnano PfV-GT8. f. CD8+ effector memory CD107a+ T-cell responses to GT8 domain in BALB/c mice immunized with DLmono_GT8, DLnano_3BVE_GT8, DLnano_LS_GT8 and DLnano_PfV-GT8 as in e. g. Humoral responses in BALB/c mice immunized with 2 μg doses of DLmono_GT8, DL_GT8_IMX313P, DLnano_3BVE_GT8, DLnano_LS_GT8 and DLnano_PfV-GT8 seven d.p.i. h. CD8+ effector memory CD107a+ T-cell responses to GT8 domain in BALB/c mice immunized twice with 2 μg DLmono_GT8, DL_GT8 IMX313P, DLnano_3BVE_GT8, DLnano_LS_GT8 and DLnano_PfV-GT8 three weeks apart. Each group contains five mice; each dot represents a mouse; error bar represents standard deviation; arrow below the plot represents an immunization; two-tailed Mann-Whitney Rank Test used to compare groups; p-values were adjusted for multiple comparison where appropriate; *, p<0.05.

FIG. 27 shows design and evaluation of new DLnano influenza hemagglutinin vaccine to determine generalizability of technology to other viral antigens. a. SECMAL trace of lectin and SEC purified LS_HA_NC99. b. nsEM image of SEC-purified fraction of in vitro produced protein LS_HA_NC99 nanoparticles. c. Humoral responses in BALB/c mice that received DLnano LS_HA_NC99 or DLmono_HA_NC99 at 1 μg dose. d. Autologous HAI titers against the H1 NC99 strain at D0, D42 (post-dose #2) and D56 (post-dose #3) for mice treated with 1 μg DLmono_HA_NC99 or DLnano_LS_HA_NC99. e. Heterologous HAI titers against the H1 SI06 strain at 56 d.p.i for mice treated with 1 μg DLmono_HA_NC99 or DLnano_LS_HA_NC99. f. CD8+ effector memory IFNγ+ T-cell responses to NC99 HA domain in naïve BALB/c mice or mice immunized with two doses of 10 μg DLmono_HA_NC99 or DLnano_LS_HA_NC99. Each group contains five mice; each dot represents a mouse; error bar represents standard deviation; arrow below the plot represents an immunization; two-tailed Mann-Whitney Rank Test used to compare groups; p-values were adjusted for multiple comparison where appropriate; *, p<0.05.

FIG. 28 shows functional evaluations of the technology in H1 A/California/07/09 lethal challenge model comparing responses induced by DNA-encoded monomer (DLmono_HA_CA09) versus DNA-launched nanoparticle DLnano_3BVE_HA_CA09. a. SEC trace for lectin-purified recombinantly produced 3BVE_HA_CA09 nanoparticles. b. nsEM image of SEC-purified 3BVE_HA_CA09 nanoparticles. c. Binding endpoint titers to HA(CA09) over time in BALB/c mice immunized with two 1 μg doses of pVAX, DLmono_HA_CA09 or DLnano_3BVE_HA_CA09 three weeks apart. d. HAI titers to the autologous A/California/07/09 strain in BALB/c mice immunized with pVAX, DLmono_HA_CA09 or DLnano_3BVE_HA_CA09 five weeks from their first vaccination. e. Percentages of vaccinated mice surviving the lethal 10LD₅₀ H1/A/California/07/09 challenge. f. Lung viral load in challenged mice at seven days post challenge or at the time of euthanasia as determined by RT-qPCR. g. Weight changes in mice immunized with pVAX, DLmono_HA_CA09 or DLnano_3BVE_HA_CA09 seven days following 10LD₅₀ H1/A/California/07/09 challenge. h. H&E stain for lung histo-pathology in mice seven days post viral challenge or at the time of euthanasia, normal lung histology is shown for comparison; scale bar represents 100 μm. Each group contains five mice; each dot represents a mouse; error bar represents standard deviation; arrow below the plot represents an immunization; two-tailed Mann-Whitney Rank Test used to compare groups; p-values were adjusted for multiple comparison where appropriate; *, p<0.05.

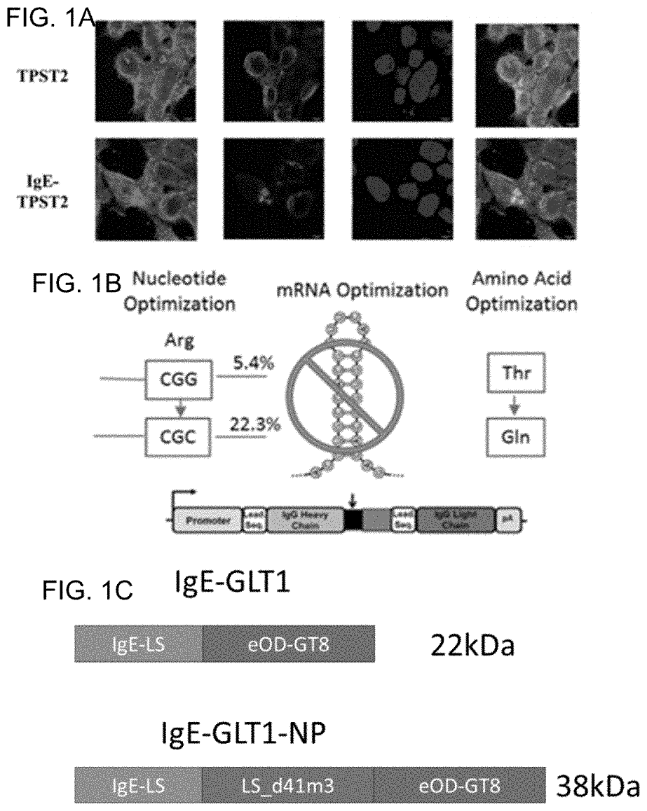

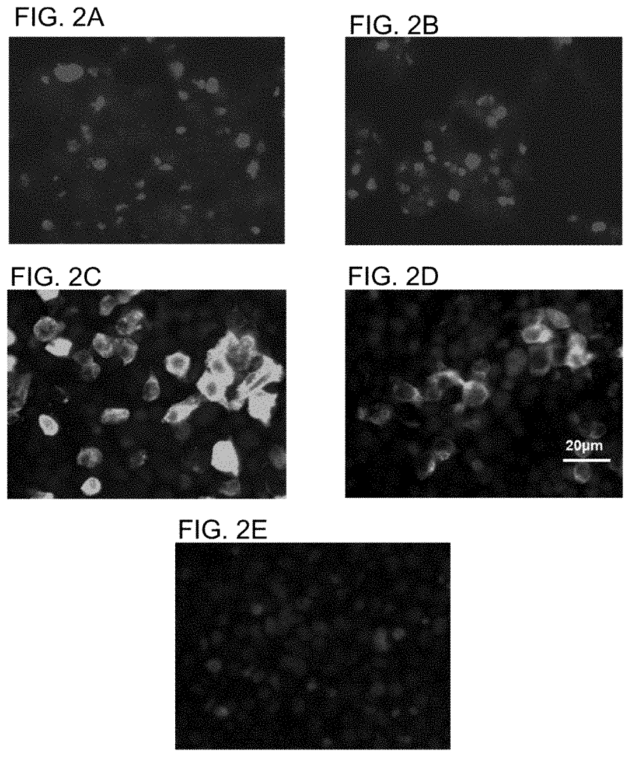

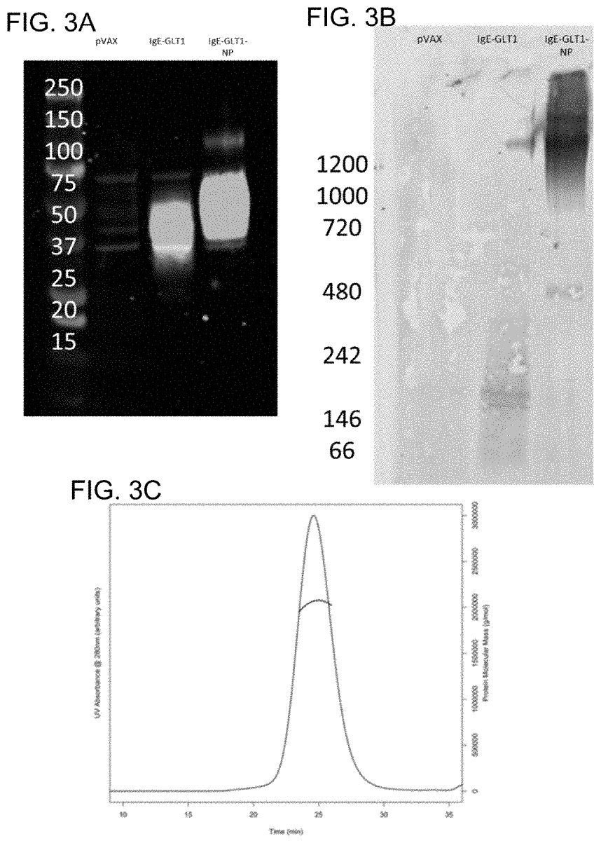

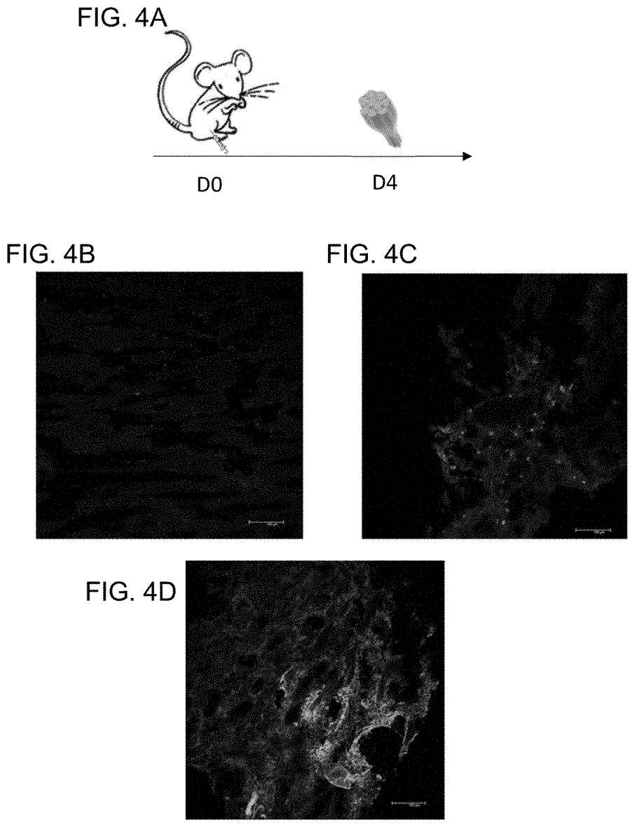

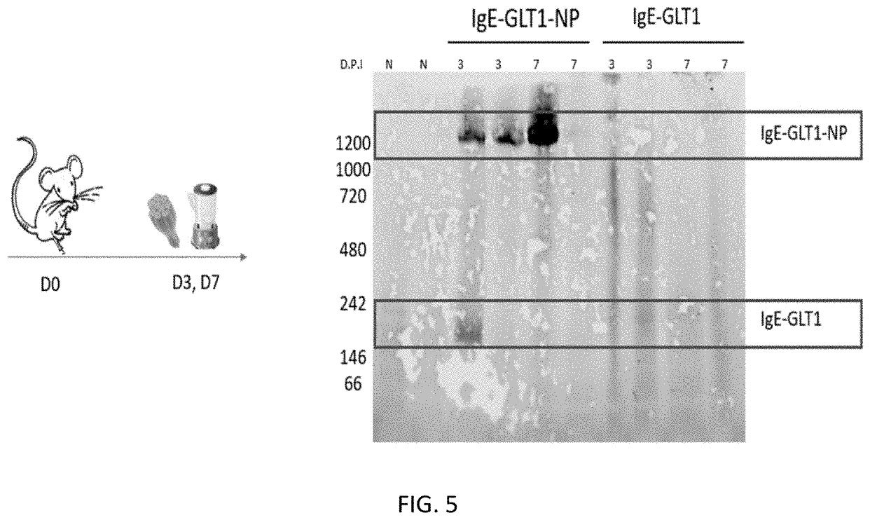

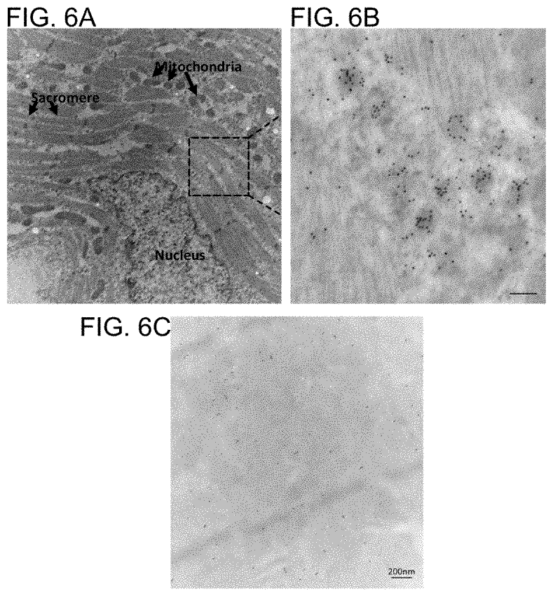

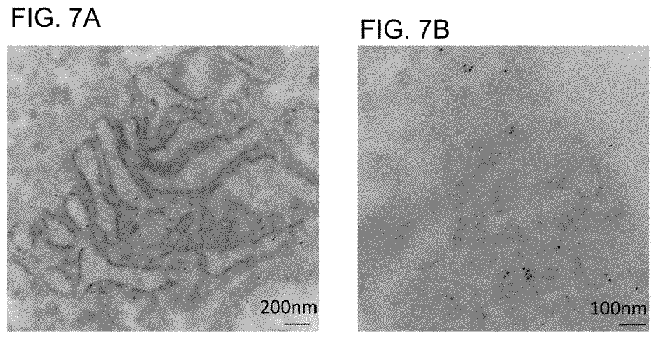

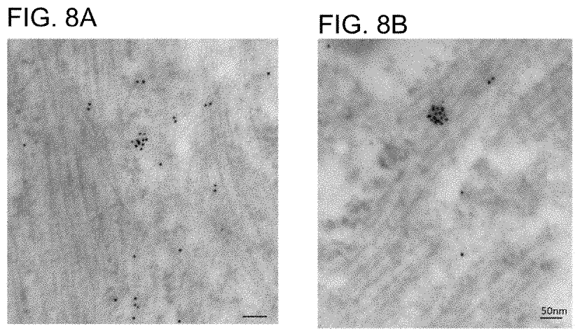

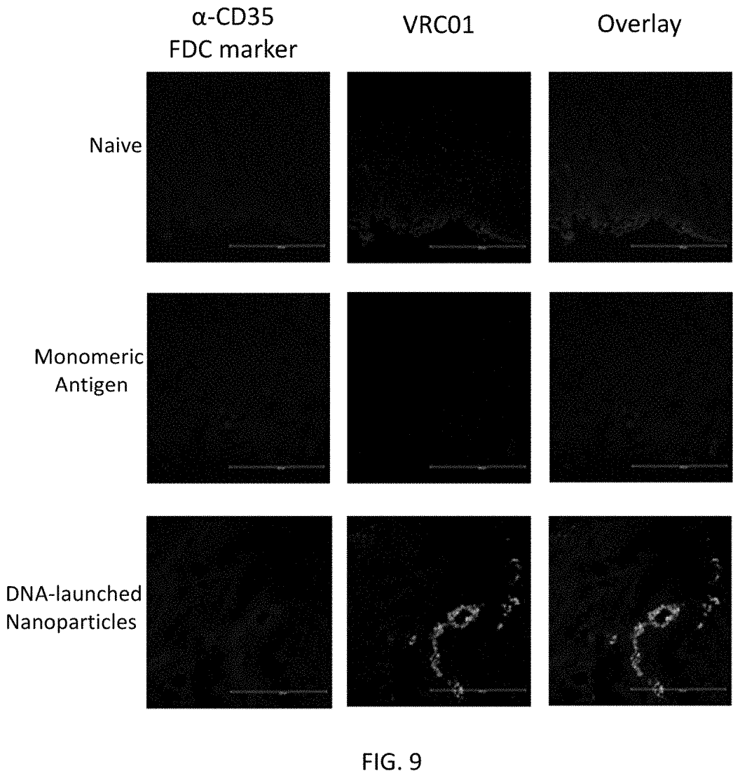

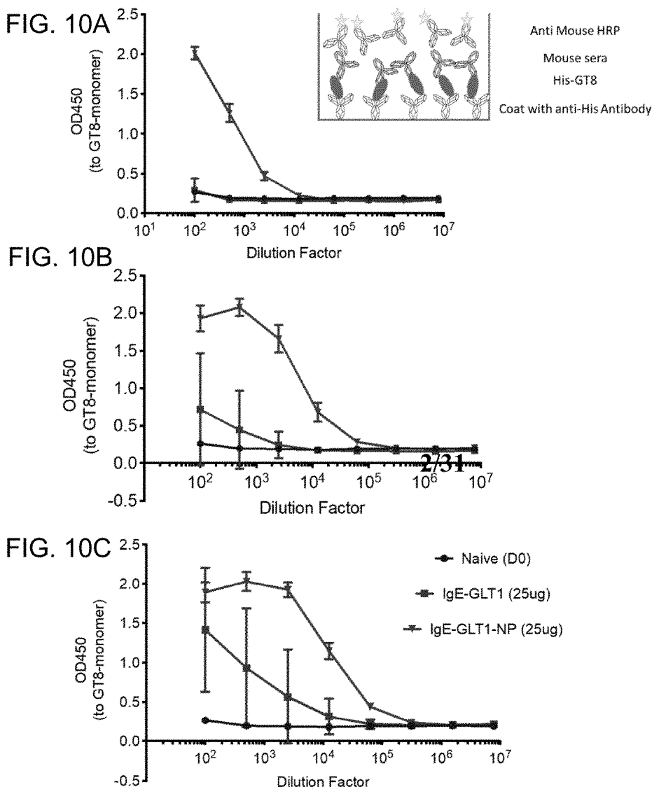

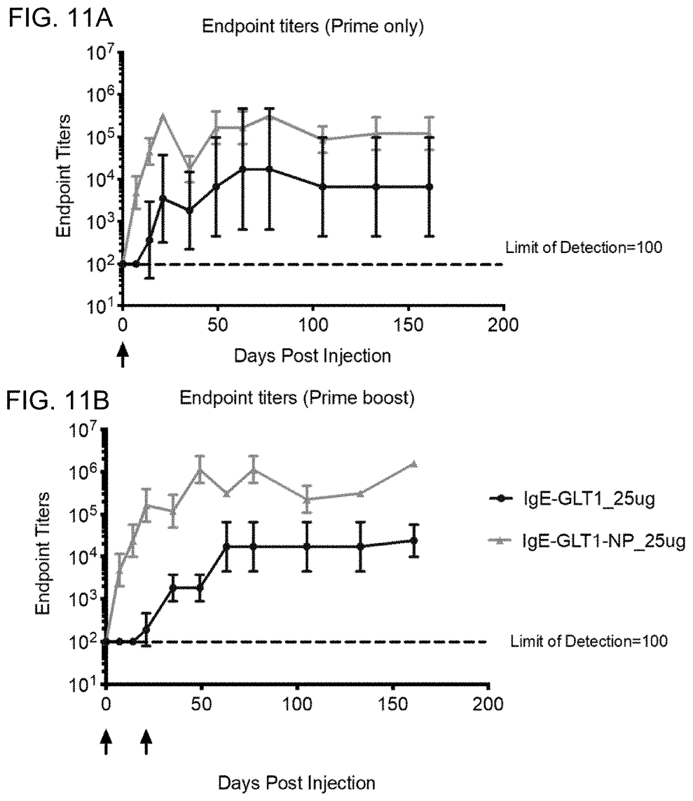

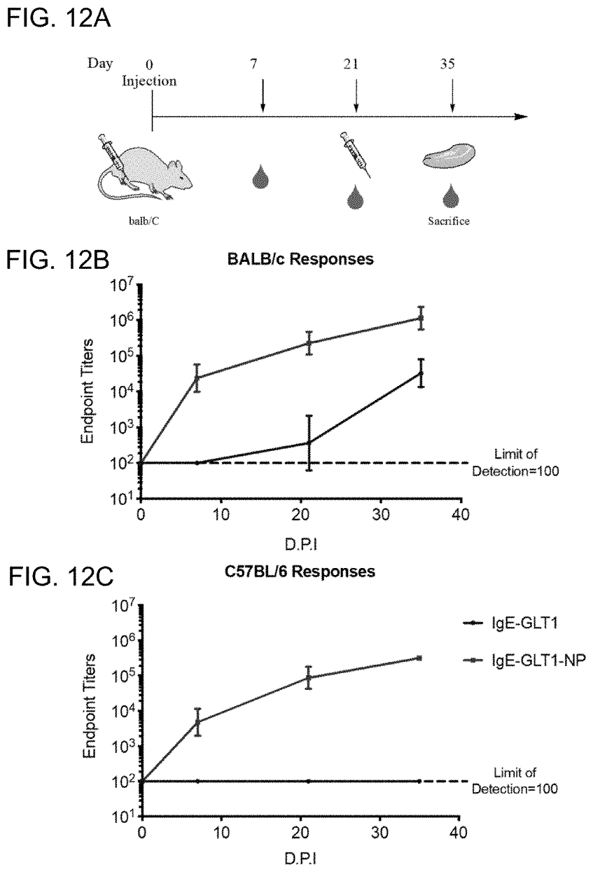

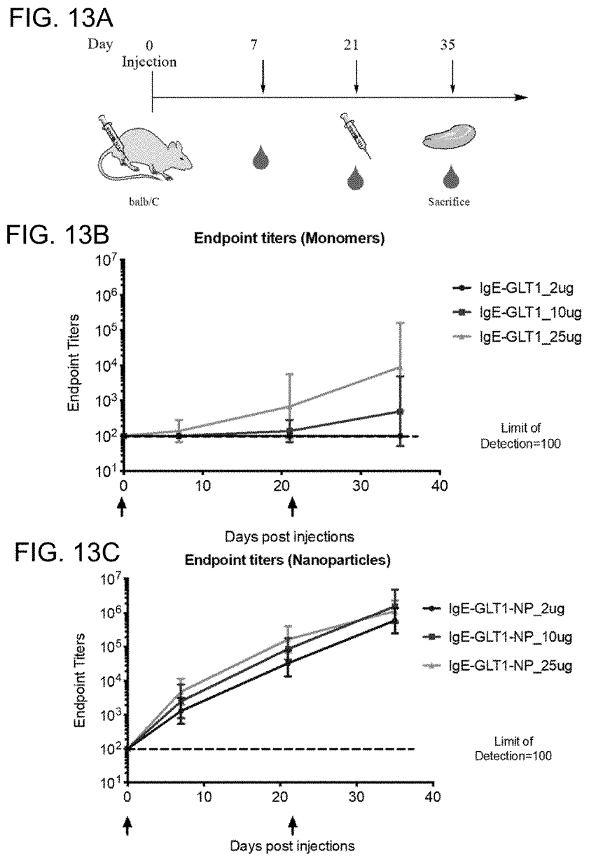

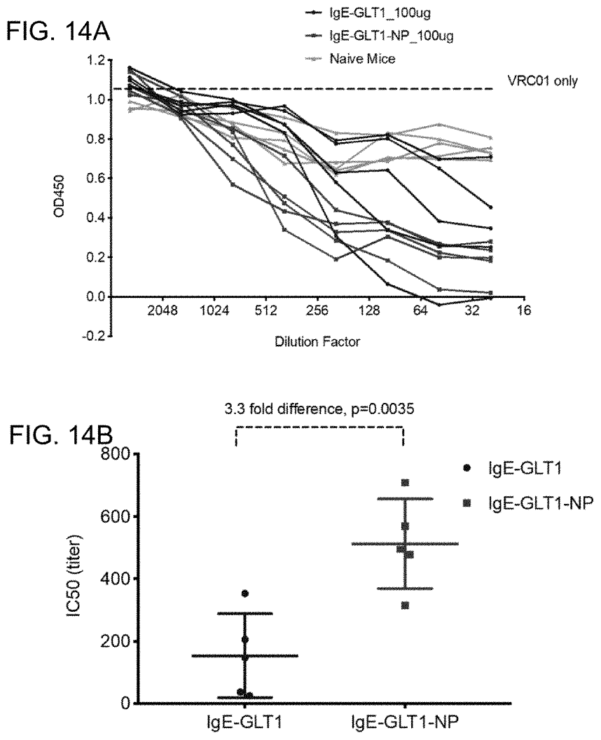

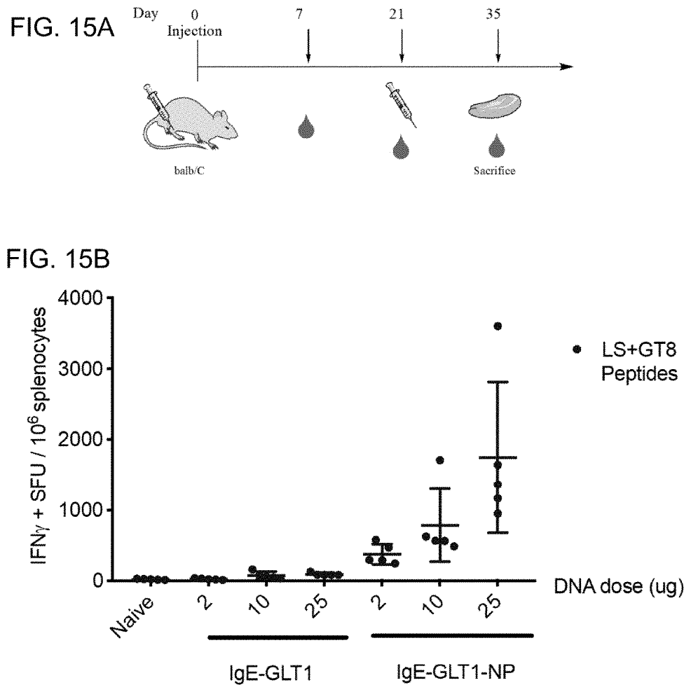

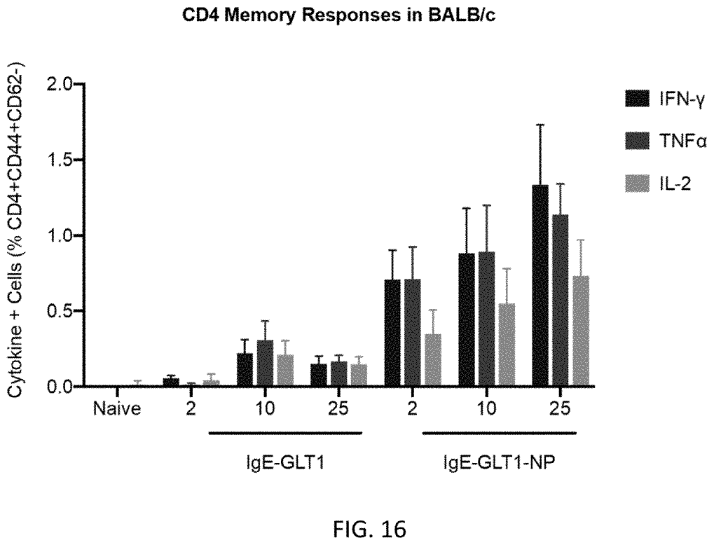

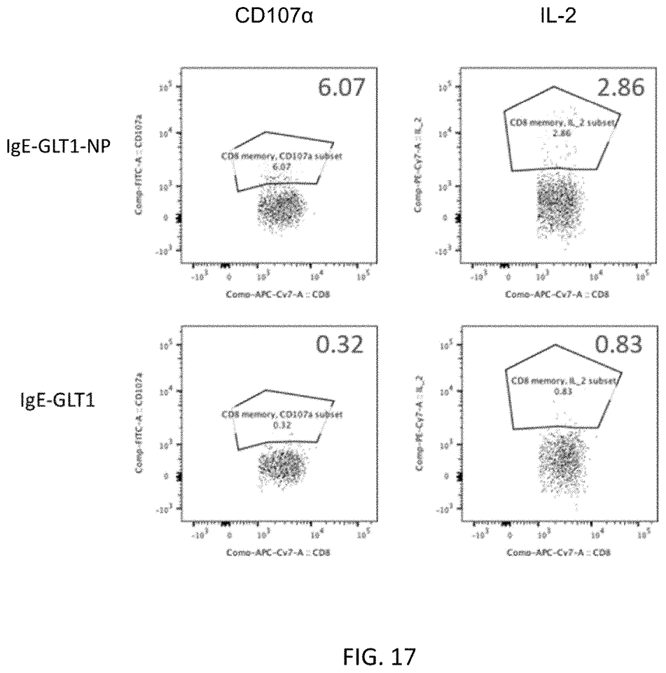

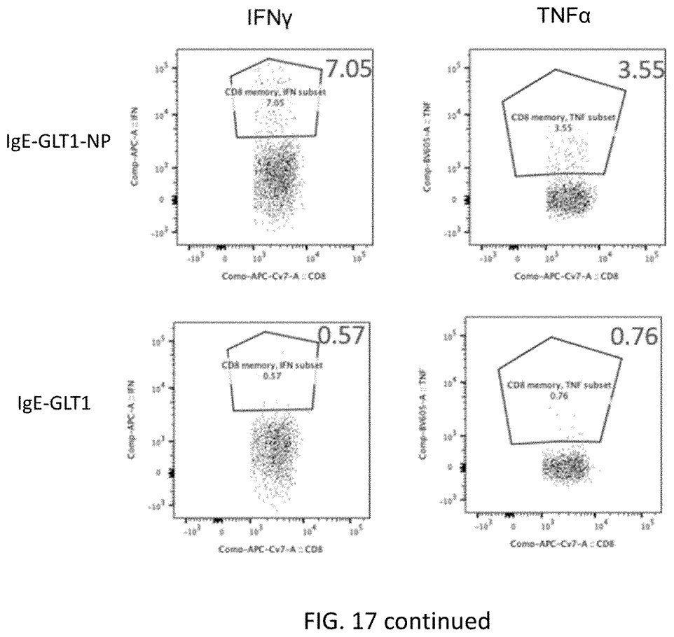

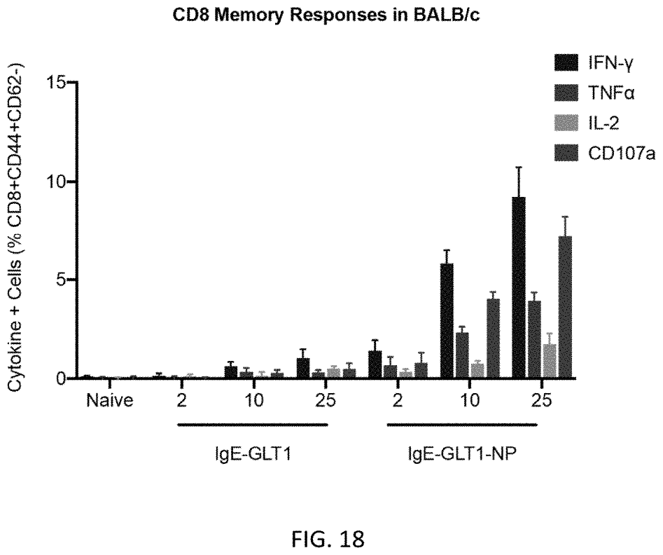

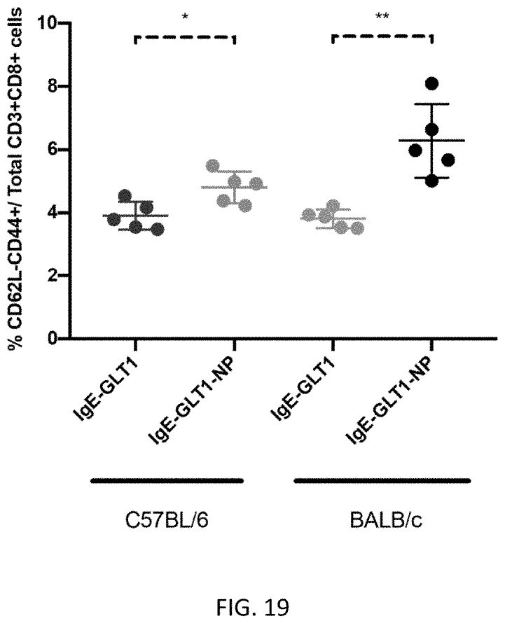

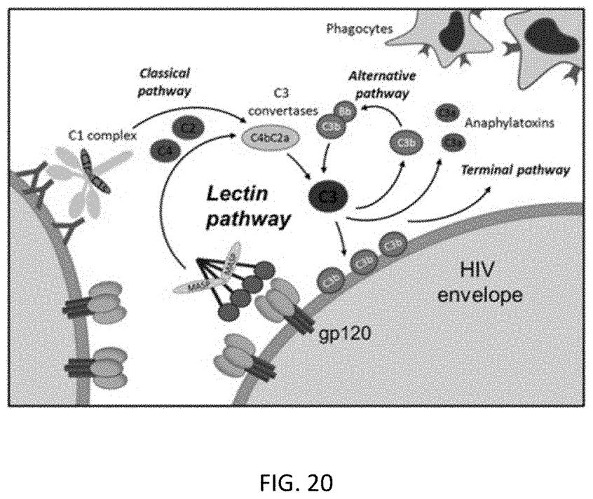

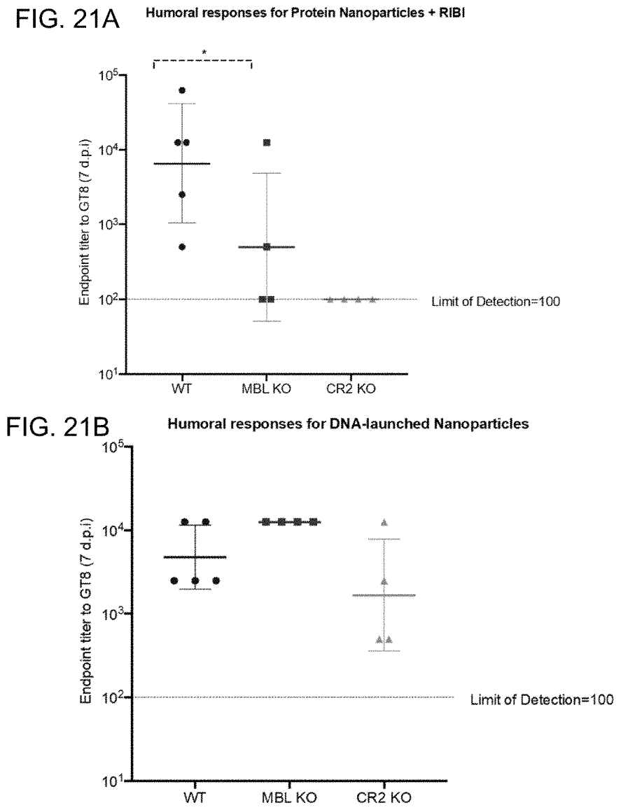

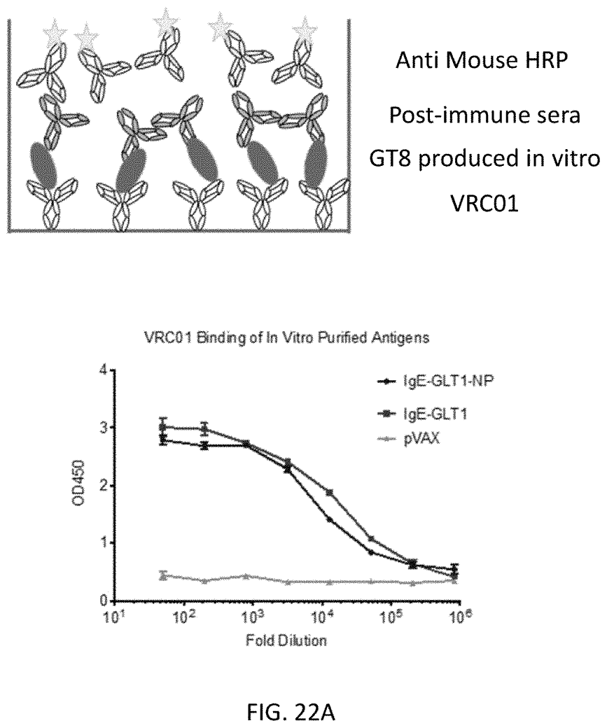

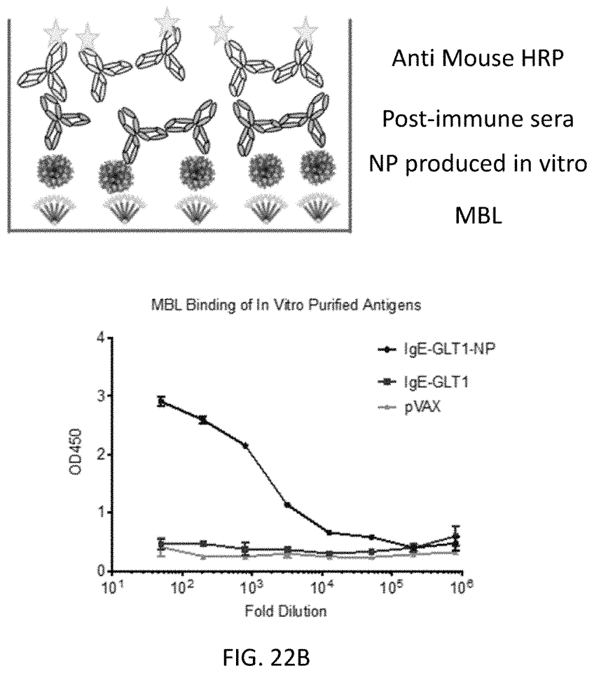

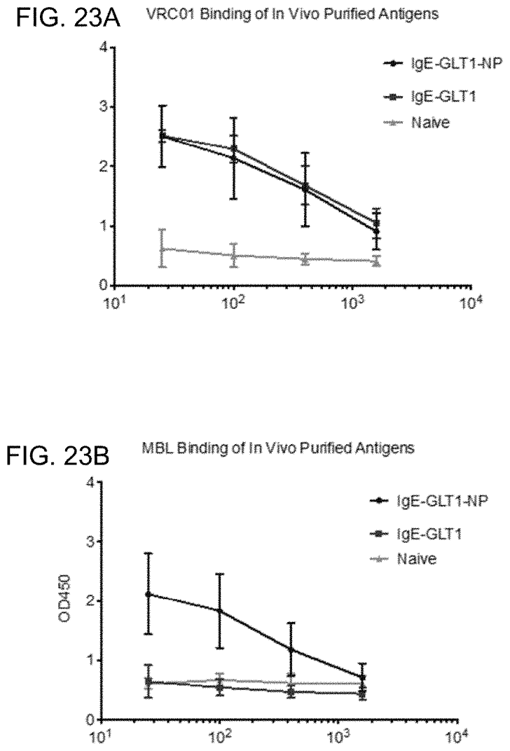

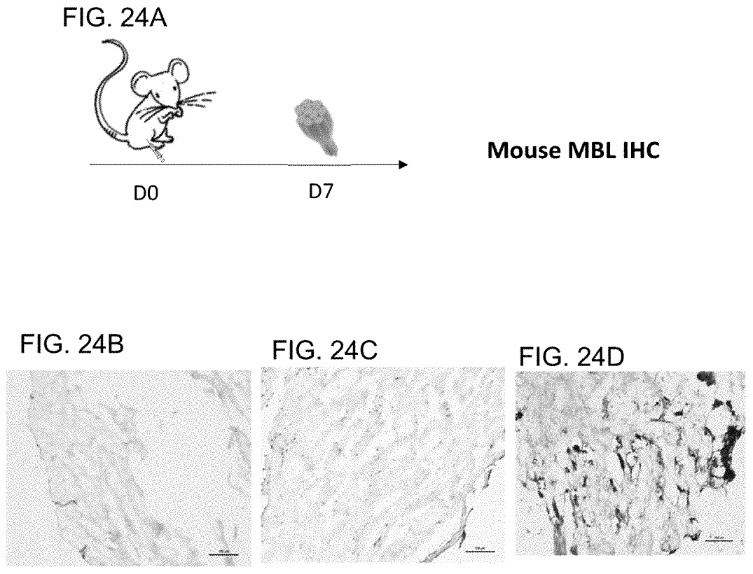

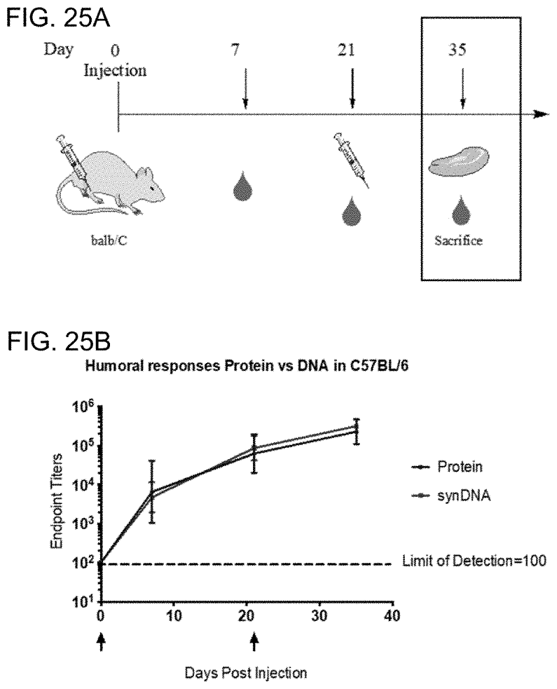

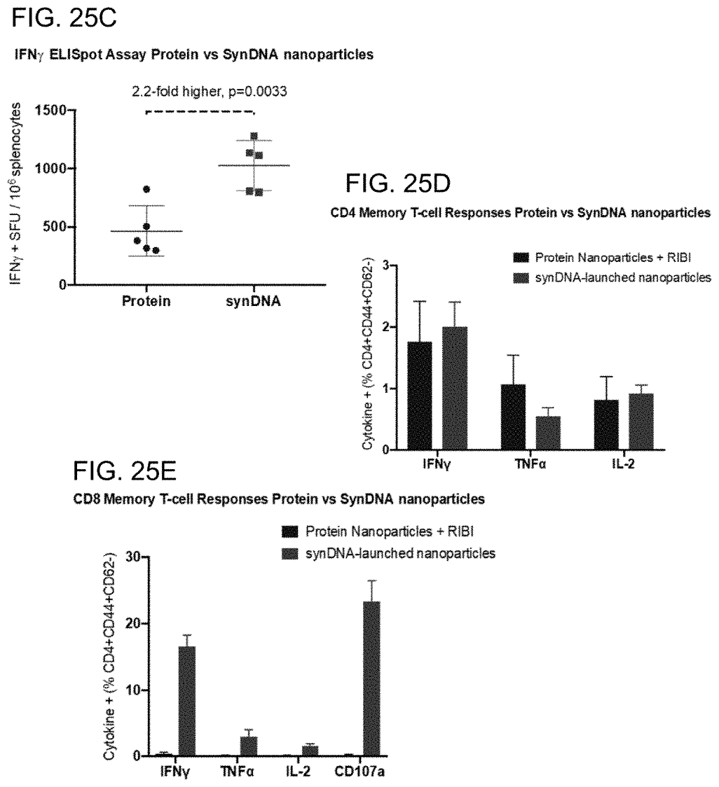

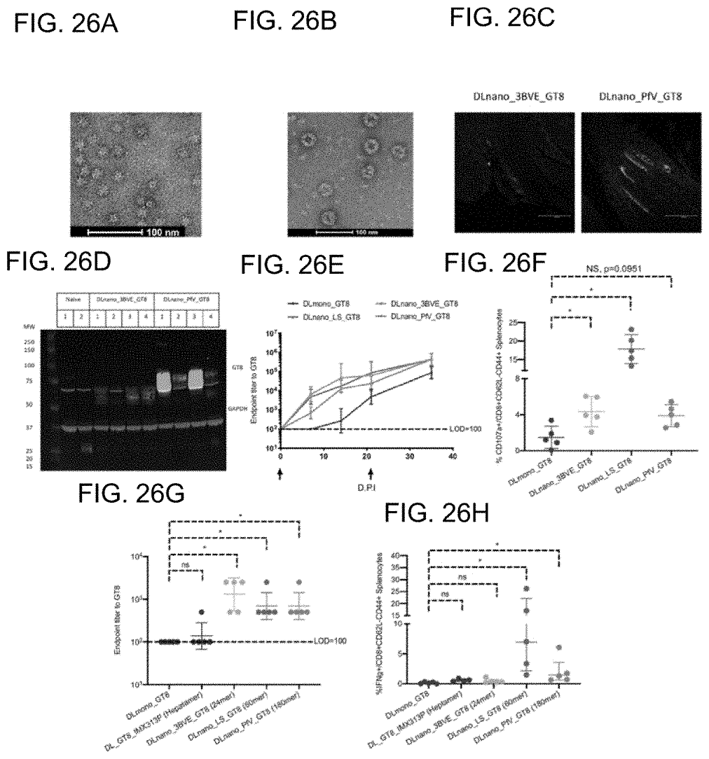

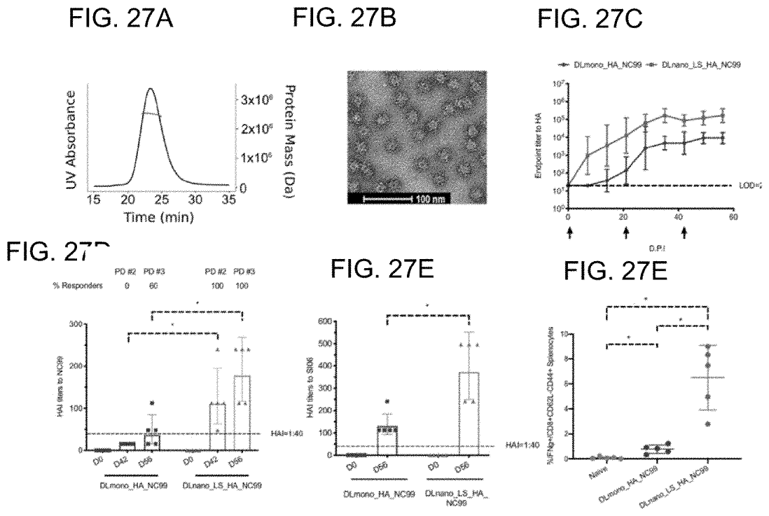

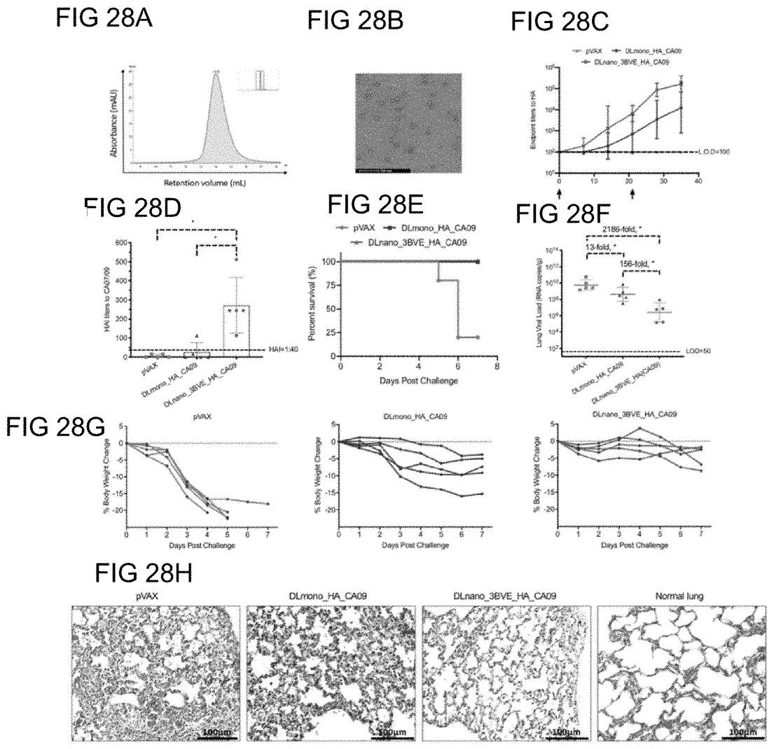

### DETAILED DESCRIPTION

The disclosed method and compositions may be understood more readily by reference to the following detailed description of particular embodiments and the Example included therein and to the FIGS. and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present disclosure which will be limited only by the appended claims.

It must be noted that as used herein and in the appended claims, the singular forms “a”, “an”, and “the” include plural reference unless the context clearly dictates otherwise. Thus, for example, reference to “a nucleic acid sequence” includes a plurality of such sequences, reference to “the nucleic acid sequence” is a reference to one or more nucleic acid sequences and equivalents thereof known to those skilled in the art, and so forth.

As used herein, the terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” in context of an immunotherapy refers to a primary response induced by ligation of a cell surface moiety. For example, in the context of receptors, such stimulation entails the ligation of a receptor and a subsequent signal transduction event. Further, the stimulation event may activate a cell and upregulate or downregulate expression or secretion of a molecule. Thus, indirect or direct ligation of cell surface moieties, even in the absence of a direct signal transduction event, may result in the reorganization of cytoskeletal structures, or in the coalescing of cell surface moieties, each of which could serve to enhance, modify, or alter subsequent cellular responses. As used herein, the terms “activating CD8+ T cells” or “CD8+ T cell activation” refer to a process (e.g., a signaling event) causing or resulting in one or more cellular responses of a CD8+ T cell (CTL), selected from: proliferation, differentiation, cytokine secretion, cytotoxic effector molecule release, cytotoxic activity, and expression of activation markers. As used herein, an “activated CD8+ T cell” refers to a CD8+ T cell that has received an activating signal, and thus demonstrates one or more cellular responses, selected from proliferation, differentiation, cytokine secretion, cytotoxic effector molecule release, cytotoxic activity, and expression of activation markers. Suitable assays to measure CD8+ T cell activation are known in the art and are described herein.

The term “combination therapy” as used herein is meant to refer to administration of one or more therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dose having a fixed ratio of each therapeutic agent or in multiple, individual doses for each of the therapeutic agents. For example, one combination of the present disclosure may comprise a pooled sample of one or more nucleic acid molecules comprising one or a plurality of expressible nucleic acid sequences and an adjuvant and/or an anti-viral agent administered at the same or different times. In some embodiments, the pharmaceutical composition of the disclosure can be formulated as a single, co-formulated pharmaceutical composition comprising one or more nucleic acid molecules comprising one or a plurality of expressible nucleic acid sequences and one or more adjuvants and/or one or more anti-viral agents. As another example, a combination of the present disclosure (e.g., DNA vaccines and anti-viral agent) may be formulated as separate pharmaceutical compositions that can be administered at the same or different time. As used herein, the term “simultaneously” is meant to refer to administration of one or more agents at the same time. For example, in certain embodiments, antiviral vaccine or immunogenic composition and antiviral agents are administered simultaneously). Simultaneously includes administration contemporaneously, that is during the same period of time. In certain embodiments, the one or more agents are administered simultaneously in the same hour, or simultaneously in the same day. Sequential or substantially simultaneous administration of each therapeutic agent can be effected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection while the other component(s) of the combination may be administered intramuscularly only. The components may be administered in any therapeutically effective sequence. A “combination” embraces groups of compounds or non-small chemical compound therapies useful as part of a combination therapy. In some embodiments, the therapeutic agent is an anti-retroviral therapy, (such as one or a combination of efavirenz, lamivudine and tenofovir disoproxil fumarate) or anti-flu therapy (such as TAMIFLU®). In some embodiments, the therapeutic agent is one or a combination of: abacavir/dolutegravir/lamivudine (Triumeq) dolutegravir/rilpivirine (Juluca), elvitegravir/cobicistat/emtricitabine/tenofovir disoproxil fumarate (Stribild), elvitegravir/cobicistat/emtricitabine/tenofovir alafenamide (Genvoya), efavirenz/emtricitabine/tenofovir disoproxil fumarate (Atripla), emtricitabine/rilpivirine/tenofovir disoproxil fumarate (Complera), emtricitabine/rilpivirine/tenofovir alafenamide (Odefsey), bictegravir, emtricitabine, and tenofovir alafenamide (Biktarvy). In some embodiments, the therapeutic agent is one or a combination of a reverse transcrioptase inhibitor of a retrovirus such as: efavirenz (Sustiva), etravirine (Intelence), nevirapine (Viramune), nevirapine extended-release (Viramune XR), rilpivirine (Edurant), delavirdine mesylate (Rescriptor). In some embodiments, the therapeutic agent is one or a combination of a protease inhibitor of a retrovirus, such as: atazanavir/cobicistat (Evotaz), darunavir/cobicistat (Prezcobix), lopinavir/ritonavir (Kaletra), ritonavir (Norvir), atazanavir (Reyataz), darunavir (Prezista), fosamprenavir (Lexiva), tipranavir (Aptivus).

As used herein, “expression” refers to the process by which a polynucleotide is transcribed from a DNA template (such as into and mRNA or other RNA transcript) and/or the process by which a transcribed mRNA (or administered mRNA) is translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “functional fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain 99%, 98%, 97%, 96%, 95%, 94%, 93%, 92%, 91%, or 90% sequence identity to the wild-type human sequence upon which the sequence is derived. In some embodiments, the functional fragment may retain 85%, 80%, 75%, 70%, 65%, or 60% sequence identity to the wild-type sequence upon which the sequence is derived.

By “fragment” is meant a portion of a polypeptide or nucleic acid molecule. This portion contains, preferably, at least about about 5%, 10%, 20%, 30%, 40%, 50%, 60%, 70%, 80%, or about 90% of the entire length of the reference nucleic acid molecule or polypeptide. A fragment may contain about 5, 10, 20, 30, 40, 50, 60, 70, 80, 90, or 100, 200, 300, 400, 500, 600, 700, 800, 900, 1000 or more nucleotides or amino acids.

“Optional” or “optionally” means that the subsequently described event, circumstance, or material may or may not occur or be present, and that the description includes instances where the event, circumstance, or material occurs or is present and instances where it does not occur or is not present.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, In some embodiments, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein an “antigen” is meant to refer to any substance that elicits an immune response.

As used herein, the term “electroporation,” “electro-permeabilization,” or “electro-kinetic enhancement” (“EP”), are used interchangeably and are meant to refer to the use of a transmembrane electric field pulse to induce microscopic pathways (pores) in a bio-membrane; their presence allows biomolecules such as plasmids, oligonucleotides, siRNA, drugs, ions, and/or water to pass from one side of the cellular membrane to the other. In some of the disclosed methods of treatment or prevention, the method comprises a step of elecoporation of a subject's tissue for a sufficient time and with a sufficient electrical field capable of inducing uptake of the pharmaceutical compositions disclosed herein into the antigen-presenting cells. In some embodiments, the cells are antigen presenting cells.

The term “pharmaceutically acceptable excipient, carrier or diluent” as used herein is meant to refer to an excipient, carrier or diluent that can be administered to a subject, together with an agent, and which does not destroy the pharmacological activity thereof and is nontoxic when administered in doses sufficient to deliver a therapeutic amount of the agent. The term “pharmaceutically acceptable salt” of nucleic acids as used herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, suifanilic, formic, toluenesulfonie, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenyiacetic, alkanoic such as acetic, HOOC—(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled viral specific antigens or polynucleotides provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the terms “prevent,” “preventing,” “prevention,” “prophylactic treatment,” and the like, are meant to refer to reducing the probability of developing a disease or condition in a subject, who does not have, but is at risk of or susceptible to developing a disease or condition.

As used herein, the term “purified” means that the polynucleotide or polypeptide or fragment, variant, or derivative thereof is substantially free of other biological material with which it is naturally associated, or free from other biological materials derived, e.g., from a recombinant host cell that has been genetically engineered to express the polypeptide of the present disclosure. That is, e.g., a purified polypeptide of the present disclosure is a polypeptide that is at least from about 70 to 100% pure, i.e., the polypeptide is present in a composition wherein the polypeptide constitutes from about 70 to about 100% by weight of the total composition. In some embodiments, the purified polypeptide of the present disclosure is from about 75% to about 99% by weight pure, from about 80% to about 99% by weight pure, from about 90 to about 99% by weight pure, or from about 95% to about 99% by weight pure.

As used herein, the terms “subject,” “individual,” “host,” and “patient,” are used interchangeably herein and refer to any mammalian subject for whom diagnosis, treatment, or therapy is desired, particularly humans. The methods described herein are applicable to both human therapy and veterinary applications. In some embodiments, the subject is a mammal, and in other embodiments the subject is a human.

The term “therapeutic effect” as used herein is meant to refer to some extent of relief of one or more of the symptoms of a disorder (e.g., HIV infection) or its associated pathology. A “therapeutically effective amount” as used herein is meant to refer to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. A “therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds of the present disclosure employed in a pharmaceutical composition at levels lower than that required in order to achieve the desired therapeutic effect and gradually increase the dosage until the desired effect is achieved.

The terms “treat,” “treated,” “treating,” “treatment,” and the like as used herein are meant to refer to reducing or ameliorating a disorder and/or symptoms associated therewith (e.g., a viral infection). “Treating” may refer to administration of the DNA vaccines described herein to a subject after the onset, or suspected onset, of a viral infection. “Treating” includes the concepts of “alleviating”, which refers to lessening the frequency of occurrence or recurrence, or the severity, of any symptoms or other ill effects related to a virus and/or the side effects associated with viral therapy. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

For any therapeutic agent described herein the therapeutically effective amount may be initially determined from preliminary in vitro studies and/or animal models. A therapeutically effective dose may also be determined from human data. The applied dose may be adjusted based on the relative bioavailability and potency of the administered agent Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below. Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained. Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In some embodiments, the nucleic acid molecules of the disclosure comprise a contiguous open reading frame encoding an antibody, or a fragment thereof, as described herein. “Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine Nucleic acids may be obtained by chemical synthesis methods or by recombinant methods. A nucleic acid will generally contain phosphodiester bonds, although nucleic acid analogs maybe included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference in their entireties. Nucleic acids containing one or more non-naturally occurring or modified nucleotides are also included within one definition of nucleic acids. The modified nucleotide analog may he located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH.sub.2, NHR, N.sub.2 or CN, wherein R is C.sub.1-C.sub.6 alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Patent Publication No. 20050107325, which are incorporated herein by reference in their entireties. Modified nucleotides and nucleic acids may also include locked nucleic acids (LNA), as described in U.S. Patent No. 20020115080, which is incorporated herein by reference. Additional modified nucleotides and nucleic acids are described in U.S. Patent Publication No. 20050182005, which is incorporated herein by reference in its entirety. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made. In some embodiments, the expressible nucleic acid sequence is in the form of DNA. In some embodiments, the expressible nucleic acid is in the form of RNA with a sequence that encodes the polypeptide sequences disclosed herein and, in some embodiments, the expressible nucleic acid sequence is an RNA/DNA hybrid molecule that encodes any one or plurality of polypeptide sequences disclosed herein.

As used herein, the term “nucleic acid molecule” is a molecule that comprises one or more nucleotide sequences that encode one or more proteins. In some embodiments, a nucleic acid molecule comprises initiation and termination signals operably linked to regulatory elements including a promoter and polyadenylation signal capable of directing expression in the cells of the individual to whom the nucleic acid molecule is administered. In some embodiments, the nucleic acid molecule also includes a plasmid containing one or more nucleotide sequences that encode one or a plurality of viral antigens. In some embodiments, the disclosure relates to a pharmaceutical composition comprising a first, second, third or more nucleic acid molecule, each of which encoding one or a plurality of viral antigens and at least one of each plasmid comprising one or more of the compositions disclosed herein.

The terms “polypeptide”, “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may he performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

By “substantially identical” is meant nucleic acid molecule (or polypeptide) exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06.

A “vector” is a nucleic acid that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide. The disclosure relates to any one or plurality of vectors that comprise nucleic acid sequences encoding any one or plurality of amino acid sequence disclosed herein.

The term “vaccine” as used herein is meant to refer to a composition for generating immunity for the prophylaxis and/or treatment of diseases (e.g., viral infections). Accordingly, vaccines are medicaments which comprise antigens in protein and/or nucleic acid forms and are intended to be used in humans or animals for generating specific defense and protective substance by vaccination. A “vaccine composition” or a “DNA vaccine composition” can include a pharmaceutically acceptable excipient, earner or diluent.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. The term “about” as used herein when referring to a measurable value such as an amount, a temporal duration, and the like, is meant to encompass variations of ±20%, ±10%, ±5%, ±1%, ±0.5%, or ±0.1% from the specified value, as such variations are appropriate to perform the disclosed methods.

“Variants” is intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule of the disclosure will have at least about 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. Variants of a particular nucleic acid molecule of the disclosure (i.e., the reference DNA sequence) can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as 1-15 amino acid residues, as few as 1-10, such as 6-10, as few as 5, as few as 4, 3, 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly.

Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

The terms “subject,” “individual,” and “patient” are used interchangeably herein to refer to a vertebrate, preferably a mammal, more preferably a human. Mammals include, but are not limited to, murines, simians, humans, farm animals, cows, pigs, goats, sheep, horses, dogs, sport animals, and pets. Tissues, cells and their progeny obtained in vivo or cultured in vitro are also encompassed by the definition of the term “subject.” The term “subject” is also used throughout the specification in some embodiments to describe an animal from which a cell sample is taken or an animal to which a disclosed cell or nucleic acid sequences have been administered. In some embodiment, the animal is a human. For treatment of those conditions which are specific for a specific subject, such as a human being, the term “patient” may be interchangeably used. In some instances in the description of the present disclosure, the term “patient” will refer to human patients suffering from a particular disease or disorder. In some embodiments, the subject may be a non-human animal from which an endothelial cell sample is isolated or provided. The term “mammal” encompasses both humans and non-humans and includes but is not limited to humans, non-human primates, canines, felines, murines, bovines, equines, caprines, and porcines.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present disclosure is not entitled to antedate such disclosure by virtue of prior disclosure. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### A. Nucleic Acid Compositions

Disclosed are compositions comprising an expressible nucleic acid sequence comprising a first nucleic acid sequence comprising a leader sequence or a pharmaceutically acceptable salt thereof; and a second nucleic acid sequence encoding a self-assembling polypeptide or a pharmaceutically acceptable salt thereof. Also disclosed are compositions comprising an expressible nucleic acid sequence comprising a first nucleic acid sequence comprising a viral antigen or a pharmaceutically acceptable salt thereof; and a second nucleic acid sequence encoding a self-assembling polypeptide or a pharmaceutically acceptable salt thereof. In some embodiments, the expressible nucleic acid comprises a leader sequence. In some embodiments, the leader is an IgE or IgG leader sequence. Additionally disclosed are compositions comprising one or a plurality of expressible nucleic acid sequences, the plurality of expressible nucleic acid sequences comprising a first nucleic acid sequence encoding a self-assembling polypeptide and a second nucleic acid sequence encoding a viral antigen and a third nucleic acid sequence encoding a leader peptide.

Disclosed are compositions comprising an expressible nucleic acid sequence comprising a first nucleic acid sequence comprising at least about 70% sequence identity to SEQ ID NO:1 or encoding a polypeptide comprising at least about 70% sequence identity to SEQ ID NO:6, or a pharmaceutically acceptable salt thereof, and a second nucleotide sequence comprising at least about 70% sequence identity to SEQ ID NO:2 or encoding a polypeptide comprising at least about 70% sequence identity to SEQ ID NO:7, or a pharmaceutically acceptable salt thereof.

Also disclosed are compositions comprising an expressible nucleic acid sequence comprising a nucleic acid sequence comprising at least about 70% sequence identity to SEQ ID NO:1 or encoding a polypeptide comprising at least about 70% sequence identity to SEQ ID NO:6, or a pharmaceutically acceptable salt thereof, and a nucleotide sequence encoding a self-assembling polypeptide. In some embodiments, the expressible nucleic acid sequence further comprises a nucleic acid sequence encoding at least one viral antigen or a pharmaceutically acceptable salt thereof. In some embodiments, the expressible nucleic acid sequence further comprises at least one nucleic acid sequence encoding a linker.

Thus, also disclosed are compositions comprising an expressible nucleic acid sequence comprising a first nucleic acid sequence comprising a leader sequence or a pharmaceutically acceptable salt thereof; and a second nucleic acid sequence comprising a nucleic acid sequence that encodes a self-assembling polypeptide or a pharmaceutically acceptable salt thereof; a third nucleic acid sequence comprising a linker sequence; and a fourth nucleic acid sequence comprising a sequence that encodes at least one viral antigen. And, in some embodiments, the expressible nucleic acid is operably linked to at least one regulatory sequence and/or forms part of a nucleic acid molecule, such as a plasmid.

In some embodiments, compositions of the disclosure relate to a composition comprising one or a plurality of expressible nucleic acid sequences, the plurality of expressible nucleic acid sequences comprising a first nucleic acid sequence encoding a self-assembling polypeptide and a second nucleic acid sequence encoding a viral antigen and, optionally, a third nucleic acid sequence encoding a leader peptide. In some embodiments the leader is an IgE or IgG leader. In some embodiments, the self-assembling polypeptide is a self-assembling peptide is expressed to envelope the antigen. Transformed or transfected cells exposed to the vaccine can produce the self-assembling peptide that envelopes the viral antigens, thereby stimulating an antigen-specific immune response against the antigen. In some embodiments, the antigen-specific immune response is a therapeutically effective immune response against the virus from which the antigen amino acid sequence is derived.

1. Leader Sequence

Disclosed are nucleic acid sequences comprising a leader sequence or a pharmaceutically acceptable salt thereof “Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

A non-limiting example of the leader sequence is the nucleic acid sequence of ATGGACTGGACCTGGATTCTGTTCCTGGTGGCCGCCGCCACAAGGGTGCACAGC (SEQ ID NO:1), which encodes the amino acid sequence of MDWTWILFLVAAATRVHS (SEQ ID NO:6). In some embodiments therefore, the leader sequence in the disclosed expressible nucleic acid sequence comprises at least about 60%, 65%, 70%, 75%, 80%, 85%, 90%, 95%, or 99% sequence identity to SEQ ID NO:1. In other embodiments, the leader sequence in the disclosed expressible nucleic acid sequence comprises the nucleic acid sequence of SEQ ID NO:1. In some embodiments therefore, the leader sequence in the disclosed expressible nucleic acid sequence encodes a polypeptide comprising at least about 60%, 65%, 70%, 75%, 80%, 85%, 90%, 95%, or 99% sequence identity to SEQ ID NO:6. In other embodiments, the leader sequence in the disclosed expressible nucleic acid sequence encodes a polypeptide comprising the amino acid sequence of SEQ ID NO:6.

Another non-limiting example of the leader sequence is the nucleic acid sequence Of ATGGACTGGACCTGGAGAATCCTGTTCCTGGTGGCCGCCGCCACCGGCACACA CGCCGATACACACTTCCCCATCTGCATCTTTTGCTGTGGCTGTTGCCATAGGTCCAAGTGTG GGATGTGCTGCAAAACT (SEQ ID NO:39). In some embodiments therefore, the leader sequence in the disclosed expressible nucleic acid sequence comprises at least about 60%, 65%, 70%, 75%, 80%, 85%, 90%, 95%, or 99% sequence identity to SEQ ID NO:39. In other embodiments, the leader sequence in the disclosed expressible nucleic acid sequence comprises the nucleic acid sequence of SEQ ID NO:39.

A yet another non-limiting example of the leader sequence is a polypeptide comprising MDWTWRILFLVAAATGTHA (SEQ ID NO:40). In some embodiments therefore, the leader sequence in the disclosed expressible nucleic acid sequence encodes a polypeptide comprising at least about 60%, 65%, 70%, 75%, 80%, 85%, 90%, 95%, or 99% sequence identity to SEQ ID NO: 40. In other embodiments, the leader sequence in the disclosed expressible nucleic acid sequence encodes a polypeptide comprising the amino acid sequence of SEQ ID NO:40.

2. Self-Assembling Polypeptide

The disclosure relates to an expressible nucleic acid sequence that encodes a self-assembling polypeptide. In some embodiments, the self assembling polypeptide is encoded by an antigen presenting cell that is transfected or transduced with a nucleic acid molecule comprising the expressible nucleic acid sequence that encodes the self-assembling polypeptide. In some embodiments, the nucleotide sequence encoding a self-assembling polypeptide comprises at least 70% sequence identity to SEQ ID NO:2 or a pharmaceutically acceptable salt thereof. SEQ ID NO:2 is the nucleic acid sequence encoding the lumazine synthase of hyperthermophilic bacterium Aquifex aeolicus and has the following sequence: ATGCAGATCTACGAAGGAAAACTGACCGCTGAGGGACTGAGGTTCGGAATTGTCGCAAGCCG CGCGAATCACGCACTGGTGGATAGGCTGGTGGAAGGCGCTATCGACGCAATTGTCCGGCACG GCGGGAGAGAGGAAGACATCACACTGGTGAGAGTCTGCGGCAGCTGGGAGATTCCCGTGGCA GCTGGAGAACTGGCTCGAAAGGAGGACATCGATGCCGTGATCGCTATTGGGGTCCTGTGCCG AGGAGCAACTCCCAGCTTCGACTACATCGCCTCAGAAGTGAGCAAGGGGCTGGCTGATCTGT CCCTGGAGCTGAGGAAACCTATCACTTTTGGCGTGATTACTGCCGACACCCTGGAACAGGCA ATCGAGGCGGCCGGCACCTGCCATGGAAACAAAGGCTGGGAAGCAGCCCTGTGCGCTATTGA GATGGCAAATCTGTTCAAATCTCTGCGA. The encoded polypeptide comprises the amino acid sequence of SEQ ID NO: 7. In some embodiments, other lumazine synthase sequences can be used. In some embodiments, the nucleotide sequence encoding a functional fragment of a self-assembling polypeptide comprising about 60%, 65%, 70%, 75%, 80%, 85%, 90%, 95%, or 99% sequence identity to SEQ ID NO:2. In some embodiments, the self-assembling polypeptide encoded by the expressible nucleic acid sequence of the present disclosure comprises at least about 60%, 65%, 70%, 75%, 80%, 85%, 90%, 95%, or 99% sequence identity to SEQ ID NO:7.

The disclosure also relates to an expressible nucleic acid sequence comprising one or a plurality of self-assembling polypeptides encoded by a first nucleic acid sequence comprising at least about 70%, 75%, 80%, 85%, 90%, 95%, or 99% sequence identity to SEQ ID NO:13 (3BVE):

GGGCTGAGTAAGGACATTATCAAGCTGCTGAACGAACAGGTGAACAAAGA
GATGCAGTCTAGCAACCTGTACATGTCCATGAGCTCCTGGTGCTATACCC
ACTCTCTGGACGGAGCAGGCCTGTTCCTGTTTGATCACGCCGCCGAGGAG
TACGAGCACGCCAAGAAGCTGATCATCTTCCTGAATGAGAACAATGTGCC
CGTGCAGCTGACCTCTATCAGCGCCCCTGAGCACAAGTTCGAGGGCCTGA
CACAGATCTTTCAGAAGGCCTACGAGCACGAGCAGCACATCTCCGAGTCT
ATCAACAATATCGTGGACCACGCCATCAAGTCCAAGGATCACGCCACATT
CAACTTTCTGCAGTGGTACGTGGCCGAGCAGCACGAGGAGGAGGTGCTGT
TTAAGGACATCCTGGATAAGATCGAGCTGATCGGCAATGAGAACCACGGG
CTGTACCTGGCAGATCAGTATGTCAAGGGCATCGCTAAGTCAAGGAAAAG
C.

The disclosure also relates to the expressible nucleic acid sequence comprising one or a plurality of self-assembling polypeptides encoded by a first nucleic acid sequence comprising at least about 70%, 75%, 80%, 85%, 90%, 95%, 99% or 100% sequence identity to SEQ ID NO:14 (RBE):

CTGAGCATTGCCCCCACACTGATTAACCGGGACAAACCCTACACCAAAGA
GGAACTGATGGAGATTCTGAGACTGGCTATTATCGCTGAGCTGGACGCCA
TCAACCTGTACGAGCAGATGGCCCGGTATTCTGAGGACGAGAATGTGCGC
AAGATCCTGCTGGATGTGGCCAGGGAGGAGAAGGCACACGTGGGAGAGTT
CATGGCCCTGCTGCTGAACCTGGACCCCGAGCAGGTGACCGAGCTGAAGG
GCGGCTTTGAGGAGGTGAAGGAGCTGACAGGCATCGAGGCCCACATCAAC
GACAATAAGAAGGAGGAGAGCAACGTGGAGTATTTCGAGAAGCTGAGATC
CGCCCTGCTGGATGGCGTGAATAAGGGCAGGAGCCTGCTGAAGCACCTGC
CTGTGACCAGGATCGAGGGCCAGAGCTTCAGAGTGGACATCATCAAGTTT
GAGGATGGCGTGCGCGTGGTGAAGCAGGAGTACAAGCCCATCCCTCTGCT
GAAGAAGAAGTTCTACGTGGGCATCAGGGAGCTGAACGACGGCACCTACG
ATGTGAGCATCGCCACAAAGGCCGGCGAGCTGCTGGTGAAGGACGAGGAG
TCCCTGGTCATCCGCGAGATCCTGTCTACAGAGGGCATCAAGAAGATGAA
GCTGAGCTCCTGGGACAATCCAGAGGAGGCCCTGAACGATCTGATGAATG
CCCTGCAGGAGGCATCTAACGCAAGCGCCGGACCATTCGGCCTGATCATC
AATCCCAAGAGATACGCCAAGCTGCTGAAGATCTATGAGAAGTCCGGCAA
GATGCTGGTGGAGGTGCTGAAGGAGATCTTCCGGGGCGGCATCATCGTGA
CCCTGAACATCGATGAGAACAAAGTGATCATCTTTGCCAACACCCCTGCC
GTGCTGGACGTGGTGGTGGGACAGGATGTGACACTGCAGGAGCTGGGACC
AGAGGGCGACGATGTGGCCTTTCTGGTGTCCGAGGCCATCGGCATCAGGA
TCAAGAATCCAGAGGCAATCGTGGTGCTGGAG.

The disclosure also relates to the expressible nucleic acid sequence comprising one or a plurality of self-assembling polypeptides encoded by a first nucleic acid sequence comprising at least about 70%, 75%, 80%, 85%, 90%, 95%, 99% or 100% sequence identity to SEQ ID NO:15 (I3):

GAGAAAGCAGCCAAAGCAGAGGAAGCAGCACGGAAGATGGAAGAACTGTT
CAAGAAGCACAAGATCGTGGCCGTGCTGAGGGCCAACTCCGTGGAGGAGG
CCAAGAAGAAGGCCCTGGCCGTGTTCCTGGGCGGCGTGCACCTGATCGAG
ATCACCTTTACAGTGCCCGACGCCGATACCGTGATCAAGGAGCTGTCTTT
CCTGAAGGAGATGGGAGCAATCATCGGAGCAGGAACCGTGACAAGCGTGG
AGCAGTGCAGAAAGGCCGTGGAGAGCGGCGCCGAGTTTATCGTGTCCCCT
CACCTGGACGAGGAGATCTCTCAGTTCTGTAAGGAGAAGGGCGTGTTTTA
CATGCCAGGCGTGATGACCCCCACAGAGCTGGTGAAGGCCATGAAGCTGG
GCCACACAATCCTGAAGCTGTTCCCTGGCGAGGTGGTGGGCCCACAGTTT
GTGAAGGCCATGAAGGGCCCCTTCCCTAATGTGAAGTTTGTGCCCACCGG
CGGCGTGAACCTGGATAACGTGTGCGAGTGGTTCAAGGCAGGCGTGCTGG
CAGTGGGCGTGGGCAGCGCCCTGGTGAAGGGCACACCCGTGGAAGTCGCT
GAGAAGGCAAAGGCATTCGTGGAAAAGATTAGGGGGTGTACTGAG.

In some embodiments, the expressible nucleic acid sequence comprises, consists essentially of, or consists of any one or plurality of the nucleic acid sequences encoding a self-assembling polypeptide comprising at least about 70%, 75%, 80%, 85%, 90%, 95%, or 99% sequence identity to SEQ ID NO:2, SEQ ID NO: 13, SEQ ID NO: 14 or SEQ ID NO:15. In some embodiments, the nucleic acid sequence encoding a self-assembling polypeptide comprises SEQ ID NO:2, SEQ ID NO: 13, SEQ ID NO: 14 or SEQ ID NO:15. In some embodiments, the expressible nucleic acid sequence of the present disclosure encodes a self-assembling polypeptide comprising at least about 70%, 75%, 80%, 85%, 90%, 95%, or 99% sequence identity to SEQ ID NO:7, SEQ ID NO:23, SEQ ID NO:31 or SEQ ID NO:26. In some embodiments, the expressible nucleic acid sequence of the present disclosure encodes a self-assembling polypeptide comprising SEQ ID NO:7, SEQ ID NO:23, SEQ ID NO:31 or SEQ ID NO:26.

3. Linker

The disclosure relates, in some embodiments, to an expressible nucleic acid sequence comprising a linker that fuses the self-assembling polypeptide to the viral antigen. In some embodiments, the expressible nucleic acid sequence comprises at least one nucleic acid sequence encoding a linker comprising at least 70% sequence identity to SEQ ID NO:3 or a pharmaceutically acceptable salt thereof. SEQ ID NO:3 is the nucleic acid sequence GGAGGCTCCGGAGGATCTGGAGGGAGTGGAGGCTCAGGAGGAGGC encoding the amino acid sequence of SEQ ID NO:8. In some embodiments, the at least one nucleic acid sequence, encoding a linker, comprises at least about 70%, 75%, 80%, 85%, 90%, 95%, or 99% sequence identity to SEQ ID NO:3 or a pharmaceutically acceptable salt thereof. In some embodiments, the at least one nucleic acid sequence, encoding a linker, comprises SEQ ID NO:3 or a pharmaceutically acceptable salt thereof. In some embodiments, the linker encoded by the expressible nucleic acid sequence of the present disclosure comprises at least about 70%, 75%, 80%, 85%, 90%, 95%, or 99% sequence identity to SEQ ID NO:8. In some embodiments, the linker encoded by the expressible nucleic acid sequence of the present disclosure comprises SEQ ID NO:8.

The disclosure also relates to an expressible nucleic acid sequence comprising one or a plurality of linker polypeptides encoded by a first nucleic acid sequence comprising at least 70%, 75%, 80%, 85%, 90%, 95%, 99% or 100% sequence identity to SEQ ID NO:16: GGCGGCTCTGGCGGAAGTGGCGGAAGTGGGGGAAGTGGAGGCGGCGGAAGCGG GGGAGGCAGCGGGGGAGGG. In some embodiments, the at least one nucleic acid sequence, encoding a linker, comprises at least about 70%, 75%, 80%, 85%, 90%, 95%, or 99% sequence identity to SEQ ID NO:16 or a pharmaceutically acceptable salt thereof. In some embodiments, the at least one nucleic acid sequence, encoding a linker, comprises SEQ ID NO:16 or a pharmaceutically acceptable salt thereof.

The disclosure also relates to an expressible nucleic acid sequence comprising one or a plurality of linker polypeptides encoded by a first nucleic acid sequence comprising at least 70%, 75%, 80%, 85%, 90%, 95%, 99% or 100% sequence identity to SEQ ID NO:17: GGCGGAAGCG GCGGAAGCGGCGGGTCT. In some embodiments, the at least one nucleic acid sequence, encoding a linker, comprises at least about 70%, 75%, 80%, 85%, 90%, 95%, or 99% sequence identity to SEQ ID NO:17 or a pharmaceutically acceptable salt thereof. In some embodiments, the at least one nucleic acid sequence, encoding a linker, comprises SEQ ID NO:17 or a pharmaceutically acceptable salt thereof.

The disclosure also relates to an expressible nucleic acid sequence comprising one or a plurality of linker polypeptides encoded by a first nucleic acid sequence comprising at least 70%, 75%, 80%, 85%, 90%, 95%, 99% or 100% sequence identity to SEQ ID NO:19: GGCGGCAGCGGCGGCAGCGGCGGGAGCGGAGGAAGT. In some embodiments, the at least one nucleic acid sequence, encoding a linker, comprises at least about 70%, 75%, 80%, 85%, 90%, 95%, or 99% sequence identity to SEQ ID NO:19 or a pharmaceutically acceptable salt thereof. In some embodiments, the at least one nucleic acid sequence, encoding a linker, comprises SEQ ID NO:19 or a pharmaceutically acceptable salt thereof.

The disclosure also relates to an expressible nucleic acid sequence comprising one or a plurality of linker polypeptides comprising at least 70%, 75%, 80%, 85%, 90%, 95%, 99% or 100% sequence identity to SEQ ID NO:22, SEQ ID NO:27 or SEQ ID NO:32. The disclosure also relates to an expressible nucleic acid sequence comprising one or a plurality of linker polypeptides comprising SEQ ID NO:22, SEQ ID NO:27 or SEQ ID NO:32.

A linker can be either flexible or rigid or a combination thereof Δn example of a flexible linker is a GGS repeat. In some embodiments, the GGS can be repeated about 1, 2, 3, 4, 5, 6, 7, 8, 9, or 10 times. An example of a rigid linker is 4QTL-115 Angstroms, single chain 3-helix bundle represented by the sequence

(SEQ ID NO: 18)
NEDDMKKLYKQMVQELEKARDRMEKLYKEMVELIQKAIELMRKIFQEVKQ
EVEKAIEEMKKLYDEAKKKIEQMIQQIKQGGDKQKMEELLKRAKEEMKKV
KDKMEKLLEKLKQIMQEAKQKMEKLLKQLKEEMKKMKEKMEKLLKEMKQR
MEEVKKKMDGDDELLEKIKKNIDDLKKIAEDLIKKAEENIKEAKKIAEQL
VKRAKQLIEKAKQVAEELIKKILQLIEKAKEIAEKVLKGLE.

In some embodiments, each linker is independently selectable from about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural nucleic acids in length.

In some embodiments, each linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length. In some embodiments, each linker is independently selectable from a linker that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length. In some embodiments, each linker is about 21 natural or non-natural nucleic acids in length.

In some embodiments, the length of each linker according to Formula I is different. For example, in some embodiments, the length of a first linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length, and the length of a second linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length, where the length of the first linker is different from the length of the second linker. Various configurations can be envisioned by the present disclosure, where Formula I comprises 1, 2, 3, 4, 5, 6, 7, 8, 9, 10 or more linkers wherein the linkers are of similar or different lengths.

In certain embodiments, two linkers can be used together, in a nucleotide sequence that encodes a fusion peptide. Accordingly, in some embodiments, the first linker is independently selectable from about 0 to about 25 natural or non-natural nucleic acids in length, about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural nucleic acids in length. In some embodiments, the second linker is independently selectable from about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural nucleic acids in length. In some embodiments, the first linker is independently selectable from a linker that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length. In some embodiments, the second linker is independently selectable from a linker that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length.

4. Viral Antigens

The disclosure relates to one or a plurality of nucleic acid molecules that comprise at least one expressible nucleic acid sequence, the expressible nucleic acid sequence comprises a first nucleic acid sequence encoding a self-assembling polypeptide and a second nucleic acid sequence encoding a viral antigen. In some embodiments, the nucleic acid molecule encodes a fusion peptide comprising one or a plurality of self-assembling peptides and one or a plurality of viral antigens. In some embodiments, upon administration to a subject, the composition comprising a nucleic acid comprising the expressible nucleic acid sequence is transfected or transduced into an antigen presenting cell which encodes the expressible nucleic acid sequence. After a plurality of expressible nucleic acid sequences are encoded, the self-assembling peptides assemble into a nanoparticle comprising the one or plurality of viral antigens. Antigen presenting cells expressing the one or plurality of viral antigens can elicit a therapeutically effective antigen-specific immune response against the virus in a subject. In some embodiments, the viral antigen can be an antigen from a Retroviridae or Flavivirus. For example, in some embodiments, the viral antigen can be an antigen from human immunodeficiency virus-1 (HIV-1). In some embodiments, the viral antigen comprises at least 70% sequence identity to SEQ ID NO: 4 or a pharmaceutically acceptable salt thereof. SEQ ID NO:4 is a fragment of gp120 represented by the nucleic acid sequence:

GACACCATCACACTGCCATGCCGCCCTGCACCACCTCCACATTGTAGCTC
CAACATCACCGGCCTGATTCTGACAAGACAGGGGGGATATAGTAACGATA
ATACCGTGATTTTCAGGCCCTCAGGAGGGGACTGGAGGGACATCGCACGA
TGCCAGATTGCTGGAACAGTGGTCTCTACTCAGCTGTTTCTGAACGGCAG
TCTGGCTGAGGAAGAGGTGGTCATCCGATCTGAAGACTGGCGGGATAATG
CAAAGTCAATTTGTGTGCAGCTGAACACAAGCGTCGAGATCAATTGCACT
GGCGCAGGGCACTGTAACATTTCTCGGGCCAAATGGAACAATACCCTGAA
GCAGATCGCCAGTAAACTGAGAGAGCAGTACGGCAATAAGACAATCATCT
TCAAGCCTTCTAGTGGAGGCGACCCAGAGTTCGTGAACCATAGCTTTAAT
TGCGGGGGAGAGTTCTTTTATTGTGATTCCACACAGCTGTTCAACAGCAC
TTGGTTTAATTCCACC

In some embodiments, disclosed are compositions comprising the IgE leader sequence and a fragment of gp120 viral antigen. For example, the nucleic acid sequence of the IgE leader sequence and the fragment of gp120 viral antigen can be:

GGATCCGCCACCATGGACTGGACCTGGATTCTGTTCCTGGTGGCCGCCGC
CACAAGGGTGCACAGCGACACCATCACACTGCCATGCCGCCCTGCACCAC
CTCCACATTGTAGCTCCAACATCACCGGCCTGATTCTGACAAGACAGGGG
GGATATAGTAACGATAATACCGTGATTTTCAGGCCCTCAGGAGGGGACTG
GAGGGACATCGCACGATGCCAGATTGCTGGAACAGTGGTCTCTACTCAGC
TGTTTCTGAACGGCAGTCTGGCTGAGGAAGAGGTGGTCATCCGATCTGAA
GACTGGCGGGATAATGCAAAGTCAATTTGTGTGCAGCTGAACACAAGCGT
CGAGATCAATTGCACTGGCGCAGGGCACTGTAACATTTCTCGGGCCAAAT
GGAACAATACCCTGAAGCAGATCGCCAGTAAACTGAGAGAGCAGTACGGC
AATAAGACAATCATCTTCAAGCCTTCTAGTGGAGGCGACCCAGAGTTCGT
GAACCATAGCTTTAATTGCGGGGGAGAGTTCTTTTATTGTGATTCCACAC
AGCTGTTCAACAGCACTIGGITTAATTCCACCTGATAACTCGAG (SEQ
ID NO: 11; refered to as IgE-GLT1).

In some embodiments, the disclosure relates to a composition or pharmaceutical composition comprising a nucleic acid molecule comprising at least one expressible nucleic acid sequence, the expressible nucleic acid sequence comprising, in the 5′ to 3′ orientation at least one leader sequence, at least one nucleic acid that encodes a self-assembling polypeptide and at least one nucleic acid that encodes at least one viral antigen, and, in each case, the nucleic acid sequences and/or nucleic acid sequence may be a pharmaceutically acceptable salt of the aforementioned molecule and/or sequence. In some embodiments, the disclosure relates to a pharmaceutical composition comprising: (i) a therapeutically effective amount of a nucleic acid molecule comprising at least one expressible nucleic acid sequence, the expressible nucleic acid sequence comprising, in the 5′ to 3′ orientation, at least one leader sequence, at least one nucleic acid that encodes a self-assembling polypeptide and at least one nucleic acid that encodes at least one viral antigen; and (ii) a pharmaceutically acceptable carrier. In any of the above embodiments, the nucleic acid sequences may also include a nucleic acid sequence that encodes a linker. The disclosure also relates to one or more prokaryotic or eukaryotic cells comprising any of the disclosed nucleic acid molecules disclosed herein.

Viral antigens are known for several genera of viruses and viral strains. In some embodiments, the compositions comprise any one or plurality of nucleic acid sequence encoding any one or plurality, in 5′ to 3′ orientation, of viral antigens that are at least 70%, 80%, 90%, 95%, or 100% sequence identity to the sequences in Table 1.

TABLE 1
West Nile Virus capsid (SEQ ID NO: 41)
ATGTCTAAGA AACCAGGAGG GCCCGGCAAG AGCCGGGCTG TCAATATGCT
AAAACGCGGAATGCCCCGCG TGTTGTCCTT GATTGGACTG AAGAGGGCTA
TGTTGAACCT GATCGACGGCAAGGGGCCAA TACGATTTGT GTTGGCTCTC
TTGGCGTTCT TCAGGTTCAC AGCAATTGCTCCGACCCGAG CAGTGCTGGA
TCGATGGAGA GGTGTGAACA AACAAACAGC GATGAAACACCTTTTGAGTT
TTAAGAAGGA ACTAGGGACC TTGACCAGTG CTATCAATCG
GCGGAGCTCAAAACAAAAGA AAAGA
HPV major capsid (SEQ ID NO: 42)
ATGTCACTTT GGCTTCCATC AGAAGCTACT GTTTACCTTC CACCAGTTCC
AGTTTCAAAA GTTGTTTCAA CTGATGAATA CGTTGCTAGG ACTAATATTT
ACTACCATGC TGGAACTTCA AGGCTTCTTG CTGTTGGACA TCCATACTTT
CCAATTAAAA AACCAAATAA TAATAAAATT CTTGTTCCAA AAGTTTCAGG
ACTTCAATAC AGGGTTTTTA GGATTCATCT TCCAGATCCA AATAAATTTG
GATTTCCAGA TACTTCATTT TACAATCCAG ATACTCAAAG
GCTTGTTTGGGCTTGTGTTG GAGTTGAAGT TGGAAGGGGA CAACCACTTG
GAGTTGGAAT TTCAGGACAT CCACTTCTTA ATAAACTTGA TGATACTGAA
AATGCTTCAG CTTACGCTGC TAATGCTGGA GTTGATAATA GGGAATGTAT
TTCAATGGAT TACAAACAAA CTCAACTTTG TCTTATTGGA TGTAAACCAC
CAATTGGAGA ACATTGGGGA AAAGGATCAC CATGTACTAA TGTTGCTGTT
AATCCAGGAG ATTGTCCACC ACTTGAACTT ATTAATACTG TTATTCAAGA
TGGAGATATGGTTGATACTG GATTTGGAGC TATGGATTTT ACTACTCTTC
AAGCTAATAA ATCAGAAGTT CCACTTGATA TCTGTACTTC AATTTGTAAA
TACCCAGATT ACATTAAAAT GGTTTCAGAA CCATACGGAG ATTCACTTTT
TTTTTACCTT AGGAGGGAAC AAATGTTTGT TAGGCATCTT TTTAATAGGG
CTGGAGCTGT TGGAGAAAAT GTTCCAGATG ATCTTTACAT TAAAGGATCA
GGATCAACTG CTAATCTTGC TTCATCAAAT TACTTTCCAA CTCCATCAGG
ATCAATGGTTACTTCAGATG CTCAAATTTT TAATAAACCA TACTGGCTTC
AAAGGGCTCA AGGACATAAT AATGGAATTT GTTGGGGAAA TCAACTTTTT
GTTACTGTTG TTGATACTAC TAGGTCAACT AATATGTCAC TTTGTGCTGC
TATTTCAACT TCAGAAACTA CTTACAAAAA TACTAATTTT AAAGAATACC
TTAGGCATGG AGAAGAATAC GATCTTCAAT TTATTTTTCA ACTTTGTAAA
ATTACTCTTA CTGCTGATGT TATGACTTAC ATTCATTCAA TGAATTCAAC
TATTCTTGAAGATTGGAATT TTGGACTTCA ACCACCACCA GGAGGAACTC
TTGAAGATAC TTACAGGTTT GTTACTTCAC AAGCTATTGC TTGTCAAAAA
CATACTCCAC CAGCTCCAAA AGAGGATCCA CTTAAAAAAT ACACTTTTTG
GGAAGTTAAT CTTAAAGAAA AATTTTCAGC AGATCTTGAT CAATTTCCAC
TTGGAAGGAA ATTTCTTCTT CAAGCTGGAC TTAAAGCTAA ACCAAAATTT
ACTCTTGGAA AAAGGAAAGC TACTCCAACT ACTTCATCAA CTTCAACTAC
TGCTAAAAGGAAAAAAAGGA AACTTTGA
HPV minor capsid (SEQ ID NO: 43)
ATGAGGCACA AGAGGAGCGC CAAGAGGACC AAGAGGGCCA GCGCCACCCA
GCTGTACAAGACCTGCAAGC AGGCCGGCAC CTGCCCCCCC GACATCATCC
CCAAGGTGGA GGGCAAGACCATCGCCGACC AGATCCTGCA GTACGGCAGC
ATGGGCGTGT TCTTCGGCGG CCTGGGCATCGGCACCGGCA GCGGCACCGG
CGGCAGGACC GGCTACATCC CCCTGGGCAC CAGGCCCCCC ACCGCCACCG
ACACCCTGGC CCCCGTGAGG CCCCCCCTGA CCGTGGACCC CGTGGGCCCC
AGCGACCCCA GCATCGTGAG CCTGGTGGAG GAGACCAGCT TCATCGACGC
CGGCGCCCCCACCAGCGTGC CCAGCATCCC CCCCGACGTG AGCGGCTTCA
GCATCACCAC CAGCACCGACACCACCCCCG CCATCCTGGA CATCAACAAC
ACCGTGACCA CCGTGACCAC CCACAACAAC CCCACCTTCA CCGACCCCAG
CGTGCTGCAG CCCCCCACCC CCGCCGAGAC CGGCGGCCAC TTCACCCTGA
GCAGCAGCAC CATCAGCACC CACAACTACG AGGAGATCCC CATGGACAC
CTTCATCGTGA GCACCAACCC CAACACCGTG ACCAGCAGCA CCCCCATCCC
CGGCAGCAGG CCCGTGGCCA GGCTGGGCCT GTACAGCAGG ACCACCCAGC
AGGTGAAGGT GGTGGACCCC GCCTTCGTGA CCACCCCCAC CAAGCTGATC
ACCTACGACA ACCCCGCCTA CGAGGGCATC GACGTGGACA ACACCCTGTA
CTTCAGCAGC AACGACAACA GCATCAACAT CGCCCCCGAC CCCGACTTCC
TGGACATCGT GGCCCTGCAC AGGCCCGCCC TGACCAGCAG GAGGACCGGC
ATCAGGTACA GCAGGATCGG CAACAAGCAG ACCCTGAGGA CCAGGAGCGG
CAAGAGCATC GGCGCCAAGG TGCACTACTA CTACGACCTG AGCACCATCG
ACCCCGCCGA GGAGATCGAGCTGCAGACCA TCACCCCCAG CACCTACACC
ACCACCAGCC ACGCCGCCAG CCCCACCAGCATCAACAACG GCCTGTACGA
CATCTACGCC GACGACTTCA TCACCGACAC CAGCACCAC CCCCGTGCCCA
GCGTGCCCAG CACCAGCCTG AGCGGCTACA TCCCCGCCAA CACCACCATC
CCCTTCGGTG GCGCCTACAA CATCCCCCTG GTGAGCGGCC CCGACATCCC
CATCAACATCACCGACCAGG CCCCCAGCCT GATCCCCATC GTGCCCGGCA
GCCCCCAGTA CACCATCATCGCCGACGCCG GCGACTTCTA CCTGCACCCC
AGCTACTACA TGCTGAGGAA GAGGAGGAA GAGGCTGCCCT ACTTCTTCAG
CGACGTGAGC CTGGCCGCCT GA

Influenza HA protein (from past patent US20180344842A1, which is incorporated by reference in its entirety)
The accession numbers are as follows: GQ323579.1 (ACS72657.1), GQ323564.1 (ACS72654.1), GQ323551.1 (ACS72652.1), GQ323530.1 (ACS72651.1), GQ323520.1 (ACS72650.1), GQ323495.1 (ACS72648.1), GQ323489.1 (ACS72647.1), GQ323486.1 (ACS72646.1), GQ323483.1 (ACS72645.1), GQ323455.1 (ACS72641.1), GQ323451.1 (ACS72640.1), GQ323443.1 (ACS72638.1), GQ293077.1 (ACS68822.1), GQ288372.1 (ACS54301.1), GQ287625.1 (ACS54262.1), GQ287627.1 (ACS54263.1), GQ287623.1 (ACS54261.1), GQ287621.1 (ACS54260.1), GQ286175.1 (ACS54258.1), GQ283488.1 (ACS50088.1), GQ280797.1 (ACS45035.1), GQ280624.1 (ACS45017.1), GQ280121.1 (ACS45189.1), GQ261277.1 (ACS34968.1), GQ253498.1 (ACS27787.1), GQ323470.1 (ACS72643.1), GQ253492.1 (ACS27780.1), FJ981613.1 (ACQ55359.1), FJ971076.1 (ACP52565.1), FJ969540.1 (ACP44189.1), FJ969511.1 (ACP44150.1), FJ969509.1 (ACP44147.1), GQ255900.1 (ACS27774.1), GQ255901.1 (ACS27775.1), FJ966974.1 (ACP41953.1), GQ261275.1 (ACS34967.1), FJ966960.1 (ACP41935.1), FJ966952.1 (ACP41926.1), FJ966082.1 (ACP41105.1), GQ255897.1 (ACS27770.1), CY041645.1 (ACS27249.1), CY041637.1 (ACS27239.1), CY041629 (ACS27229.1), GQ323446.1 (ACS72639.1), CY041597.1 (ACS27189.1), CY041581.1 (ACS14726.1), CY040653.1 (ACS14666.1), CY041573.1 (ACS14716.1), CY041565.1 (ACS14706.1), CY041541.1 (ACS14676.1), GQ258462.1 (ACS34667.1), CY041557.1 (ACS14696.1), CY041549.1 (ACS14686.1), GQ283484.1 (ACS50084.1), GQ283493.1 (ACS50095.1), GQ303340.1 (ACS71656.1), GQ287619.1 (ACS54259.1), GQ267839.1 (ACS36632.1), GQ268003.1 (ACS36645.1), CY041621.1 (ACS27219.1), CY041613.1 (ACS27209.1), CY041605.1 (ACS27199.1), FJ966959.1 (ACP41934.1), FJ966982.1 (ACP41963.1), CY039527.2 (ACQ45338.1), FJ981612.1 (ACQ55358.1), FJ981615.1 (ACQ55361.1), FJ982430.1 (ACQ59195.1), FJ998208.1 (ACQ73386.1), GQ259909.1 (ACS34705.1), GQ261272.1 (ACS34966.1), GQ287621.1 (ACS54260.1), GQ290059.1 (ACS66821.1), GQ323464.1 (ACS72642.1), GQ323473.1 (ACS72644.1), GQ323509.1 (ACS72649.1), GQ323560.1 (ACS72653.1), GQ323574.1 (ACS72655.1), and GQ323576.1 (ACS72656.1).

Hemagglutinin (partial) from Influenza A virus (A/New CaLedonia/20/1999(H1N1))
(SEQ ID NO: 65)
FTATYADTICIGYHANNSTDTVDTVLEKNVTVTHSVNLLEDSHNGKLCLLKGIAPLQLGNCS
VAGWILGNPECELLISKESWSYIVETPNPENGTCYPGYFADYEELREQLSSVSSFERFEIFP
KESSWPNHTVTGVSASCSHNGKSSFYRNLLWLTGKNGLYPNLSKSYVNNKEKEVLVLWGVHH
PPNIGNQRALYHTENAYVSVVSSHYSRRFTPELAKRPKVRDQEGRINYYWTLLEPGDTIIFE
ANGNLIAPWYAFALSRGFGSGIITSNAPMDECDAKCQTPQGAINSSLPFQNVHPVTIGECPK
YVRSAKLRMVTGLRNIH
Influenza A virus (A/West Virginia/01/2009(H1N1)) segment 4 hemagglutinin (HA)
(SEQ ID NO: 66)
MKAILVVLLYTFATANADTLCIGYHANNSTDTVDTVLEKNVTVTHSVNLLEDKHNGKLCKLR
GVAPLHLGKCNIAGWILGNPECESLSTASSWSYIVETSSSDNGTCYPGDFIDYEELREQLSS
VSSFERFEIFPKTSSWPNHDSNKGVTAACPHAGAKSFYKNLIWLVKKGNSYPKLSKSYINDK
GKEVLVLWGIHHPSTSADQQSLYQNADAYVFVGTSRYSKKFKPEIAIRPKVRDQEGRMNYYW
TLVEPGDKITFEATGNLVVPRYAFAMERNAGSGIIISDTPVHDCNTTCQTPKGAINTSLPFQ
NIHPITIGKCPKYVKSTKLRLATGLRNVPSIQSRGLFGAIAGFIEGGWTGMVDGWYGYHHQN
EQGSGYAADLKSTQNAIDEITNKVNSVIEKMNTQFTAVGKEFNHLEKRIENLNKKVDDGFLD
IWTYNAELLVLLENERTLDYHDSNVKNLYEKVRSQLKNNAKEIGNGCFEFYHKCDNTCMESV
KNGTYDYPKYSEEAKLNREEIDGVKLESTRIYQILAIYSTVASSLVLVVSLGAISFWMCSNG
SLQCRICI
Hemagglutinin [Influenza A virus (A/California/04/2009(H1N1))]
(SEQ ID NO: 67)
MKAILVVLLYTFATANADTLCIGYHANNSTDTVDTVLEKNVTVTHSVNLLEDKHNGKLCKLR
GVAPLHLGKCNIAGWILGNPECESLSTASSWSYIVETPSSDNGTCYPGDFIDYEELREQLSS
VSSFERFEIFPKTSSWPNHDSNKGVTAACPHAGAKSFYKNLIWLVKKGNSYPKLSKSYINDK
GKEVLVLWGIHHPSTSADQQSLYQNADTYVFVGSSRYSKKFKPEIAIRPKVRDQEGRMNYYW
TLVEPGDKITFEATGNLVVPRYAFAMERNAGSGIIISDTPVHDCNTTCQTPKGAINTSLPFQ
NIHPITIGKCPKYVKSTKLRLATGLRNIPSIQSRGLFGAIAGFIEGGWTGMVDGWYGYHHQN
EQGSGYAADLKSTQNAIDEITNKVNSVIEKMNTQFTAVGKEFNHLEKRIENLNKKVDDGFLD
IWTYNAELLVLLENERTLDYHDSNVKNLYEKVRSQLKNNAKEIGNGCFEFYHKCDNTCMESV
KNGTYDYPKYSEEAKLNREEIDGVKLESTRIYQILAIYSTVASSLVLVVSLGAISFWMCSNG
SLQCRICI
RSV (from US20180346522A1)
F immunogen DNA sequence
(SEQ ID NO: 44)
GAGCTGCCCATCCTGAAAACAAACGCCATCACCACCATCCTGGCCGCCGTGACCCTGTGCTT
CGCCAGCAGCCAGAACATCACCGAGGAATTCTACCAGAGCACCTGTAGCGCCGTGTCCAAGG
GCTACCTGTCTGCCCTGCGGACCGGCTGGTACACCAGCGTGATCACCATCGAGCTGAGCAAC
ATCAAAGAAAACAAGTGCAACGGCACCGACGCCAAAGTGAAGCTGATCAAGCAGGAACTGGA
CAAGTACAAGAACGCCGTGACCGAGCTGCAGCTGCTGATGCAGAGCACCCCTGCCGCCAACA
ACAGAGCCAGACGCGAGCTGCCCCGGTTCATGAACTACACCCTGAACAACACCAAGAACACC
AACGTGACCCTGAGCAAGAAGCGGAAGCGGCGGTTCCTGGGATTCCTGCTGGGCGTGGGCAG
CGCCATTGCCTCTGGAATCGCTGTGTCTAAGGTGCTGCACCTGGAAGGCGAAGTGAACAAGA
TCAAGTCCGCCCTGCTGAGCACCAACAAGGCCGTGGTGTCCCTGAGCAACGGCGTGTCCGTG
CTGACCAGCAAGGTGCTGGATCTGAAGAACTACATCGACAAGCAGCTGCTGCCTATCGTGAA
CAAGCAGAGCTGCAGCATCAGCAACATCGAGACAGTGATCGAGTTCCAGCAGAAGAACAACC
GGCTGCTGGAAATCACCCGCGAGTTCAGCGTGAACGCCGGCGTGACCACCCCCGTGTCCACC
TACATGCTGACCAACAGCGAGCTGCTGAGCCTGATCAACGACATGCCCATCACCAACGACCA
GAAAAAGCTGATGAGCAACAACGTGCAGATCGTGCGGCAGCAGAGCTACTCCATCATGTCCA
TCATCAAAGAAGAGGTGCTGGCCTACGTGGTGCAGCTGCCCCTGTACGGCGTGATCGACACC
CCCTGCTGGAAGCTGCACACCAGCCCCCTGTGCACCACCAACACCAAAGAGGGCAGCAACAT
CTGCCTGACCCGGACCGACCGGGGCTGGTACTGEGATAATGCCGGCAGCGTGTCATTCTTTC
CACAGGCEGAGACATGCAAGGTGCAGAGCAACCGGGTGTTCTGCGACACCATGAACAGCCTG
ACCCTGCCCTCCGAAGTGAACCTGTGCAACATCGACATCTTCAACCCTAAGTACGACTGCAA
GATOATGACCTCCAAGACCGACGTGTCCAGCTCCGTGATCACCTCCCTGGGCGCCATCGTGT
CCTGCTACGGCAAGACCAAGTGCACCGCCAGCAACAAGAACCGGGGCATCATCAAGACCTTC
AGCAACGGCTGCGACTACGTGTCCAACAAGGGGGTGGACACCGTGTCCGTGGGCAACACCCT
GTACTACGTGAACAAACAGGAAGGCAAGAGCCTGTACGTGAAGGGCGAGCCCATCATCAACT
TCTACGACCCCCTGGTGTTCCCCAGCGACGAGTTCGACGCCAGCATCAGCCAGGTGAACGAG
AAGATCAACCAGAGCCTGGCCTTCATCAGAAAGAGCGACGAGCTGCTGCACAATGTGAATGC
CGGCAAGAGCACCACCAATATCATGATCACCACAATCATCATCGTGATCATTGTGATCCTGC
TGTCCCTGATCGCCGTGGGCCTGCTGCTGTACTGCAAGGCCCGGTCCACCCCTGTGACCCTG
TCCAAGGACCAGCTGAGCGGAATCATCAACAATATCGCCTTCTCCAACTQA
Encoded protein sequence
(SEQ ID NO: 45)
MQSTPAANNRARRELPRFMNYTLNNTKNTNVTLSKKRKRRFLGFLLGVGSAIASGIAVSKVL
HLEGEVNKIKSALLSTNKAVVSLSNGVSVLTSKVLDLKNYIDKQLLPIVNKQSCSISNIETV
IEFQQKNNRLLEITREFSVNAGVTTPVSTYMLTNSELLSLINDMPITNDQKKLMSNNVQIVR
QQSYSIMSIIKEEVLAYVVQLPLYGVIDTPCWKLHTSPLCTTNTKEGSNICLTRTDRGWYXD
NAGSVSFFPQXETCKVQSNRVFCDTMNSLTLPSEVNLCNIDIFNPKYDCKXMTSKTDVSSSV
ITSLGAIVSCYGKTKCTASNKNRGIIKTFSNGCDYVSNKGVDTVSVGNTLYYVNKQEGKSLY
VKGEPIINFYDPLVFPSDEFDASISQVNEKINQSLAFIRKSDELLHNVNAGKSTTNIMITTI
IIVIIVILLSLIAVGLLLYCKARSTPVTLSKDQLSGIINNIAFSN
RSV Ga
DNA sequence of Ga
(SEQ ID NO: 46)
ATGTCCAAGAATAAGGATCAGAGGACCGCGAAAACGCTTGAGAGGACGTGGGACACGCTGAA
CCACCTCCTGTTCATCTCCTCGTGTCTCTACAAGCTCAACCTTAAGTCCATCGCGCAGATCA
CCTTGAGCATTCTCGCCATGATCATCTCCACCAGCCTTATCATTGCCGCAATCATCTTCATC
GCATCCGCCAACCATAAGGTGACATTGACTACAGCGATTATCCAAGACGCTACTAGCCAGAT
CAAGAATACCACGCCGACCTATTTGACGCAAAATCCTCAGTTGGGAATTAGCTTCTCGAATC
TCTCGGAAACCACGTCGCAGCCGACTACAATTCTTGCGTCAACGACTCCATCGGCCAAATCA
ACACCACAATCGACTACCGTAAAAACGAAGAACACGACTACAACACAGATTCAGCCTTCAAA
GCCCACGACCAAACAGAGACAGAATAAGCCGCCCAACAAGCCCAACAATGATTTTCACTTCG
AGGTGTTTAACTTCGTGCCCTGTTCGATTTGCAGCAATAACCCCACGTGCTGGGCGATTTGC
AAGCGAATCCCGAATAAGAAGCCCGGGAAAAAGACCACGACGAAACCGACAAAGAAGCCGAC
AATCAAGACAACGAAAAAGGATCTTAAACCTCAGACGACAAAGCCTAAGGAAGTCTTGACAA
CGAAGCCTACGGAAAAACCCACTATCAATACTACCAAGACTAACATCCGGACAACACTGCTG
ACGAGCAATACCACGGGAAACCCGGAGCTCACATCGCAGAAAGAGACACTCCATTCGACATC
CTCCGAGGGTAACCCTTCGCCCAGCCAGGTGTATACGACGTCAGAATACCCTAGCCAACCCT
CATCGCCCTCAAATACGACCCGGCAATGA
Protein sequence for Ga
(SEQ ID NO: 47)
MSKNKDQRTAKTLERTWDTLNHLLFISSCLYKLNLKSIAQITLSILAMIISTSLIIAAIIFI
ASANHKVTLTTAIIQDATSQIKNTTPTYLTQNPQLGISFSNLSETTSQPTTILASTTPSAKS
TPQSTTVKTKNTTTTQIQPSKPTTKQRQNKPPNKPNNDFHFEVFNFVPCSICSNNPTCWAIC
KRIPNKKPGKKTTTKPTKKPTIKTTKKDLKPQTTKPKEVLTTKPTEKPTINTTKTNIRTTLL
TSNTTGNPELTSQKETLHSTSSEGNPSPSQVYTTSEYPSQPSSPSNTTRQ
RSV Gb
DNA sequence of Gb
(SEQ ID NO: 48)
ATGAGCAAAAACAAAAACCAAAGGACGGCTCGGACGOTTGAGAAAACATGGGACACGCTTAA
TCACCTTATTGTGATCTCATCGTGTTTGTACCGGTTGAATCTCAAGAGCATCGCCCAGATTG
CGCTGTCAGTCCTGGCCATGATTATCTCGACATCACTCATCATCGCAGOCATCATCTTTATC
ATTTCAGCGAATCACAAGGTAACGCTTACAACAGTCACGGTGCAGACCATCAAGAATCATAC
CGAAAAGAATATCACAACCTACCTCACCCAAGTCAGOCCGGAGAGAGTAAGCCCCTCAAAAC
AGCCTACTACGACACCTCCCATCCACACGAACTCGGCGACCATCTCACCGAATACCAAATCA
GAAACGCATCATACGACCGCACAGACAAAGGGACGAACCACTACACCCACACAGAACAACAA
ACCCAGCACCAAGCCGAGGCCAAAGAATCCGCCCAAGAAGCCGAAAGATGACTATCACTTTG
AAGTGTTCAACTTCGTACCGTGTTCGATTTGCGGGAATAATCAGTTGTGCAAATCCATTTGC
AAGACGATCCCATCCAACAAACCGAAGAAGAAACCTACCATCAAGCCCACAAACAAGCCAAC
GACAAAAACAACGAACAAGCGCGATCCCAAAACGCTCGCGAAAACGttGAAGAAGGAAACGA
CGACAAACCCTACGAAGAAACCCACGCCCAAGACCACTGAGAGAGACACCTCCACCTCGCAA
TCGACGGTACTTGACACGACTACGAGCAAGCACACTATCCAGCAACAGTCCCTGCACTCAAC
CACGCCCGAGAATACACCAAACTCAACACAGACTCCGACAGCTTCAGAGCCTTCCACTTCGA
ATTCCACATGA
Protein sequence of Gb
(SEQ ID NO: 49)
MSKNKNQRTARTXEKTWDTXNHLIVISSCLYRLNLKSIAQIALSVLAMIISTSLIIAXIIFI
ISANHKVTLTTVTVQTIKNHTEKNITTYLTQVXPERVSPSKQPTTTPPIHTNSATISPNTKS
ETHHTTAQTKGRTTTPTQNNKPSTKPRPKNPPKKPKDDYHFEVFNFVPCSICGNNQLCKSIC
KTIPSNKPKKKPTIKPINKPTTKTTNKRDPKTLAKTLKKETTTNPTKKPTPKTTERDTSTSQ
STVLDTTTSKHTIQQQSLHSTTPENTPNSTQTPTASEPSTSNST
Filoviruses (from US20180344840A1, which is incorporated by reference in its entirety)
DNA sequence of Zaire ebolavirus glycoprotein consensus
(SEQ ID NO: 50)
ATGGGGGTCACTGGGATTCTGCAGCTGCCTAGAGATCGCTTCAAGCGAACCTCTTTCTTTCT
GTGGGTCATCATTCTGTTCCAGAGGACTTTTAGTATCCCTCTGGGCGTCATTCACAATTCTA
CCCTGCAGGTGAGTGACGTCGATAAGCTGGTGTGTCGGGACAAACTGAGCTCCACCAACCAG
CTGAGATCTGTCGGCCTGAATCTGGAGGGGAACGGAGTGGCTACCGATGTCCCAAGTGCAAC
AAAGAGATGGGGGTTTCGCTCAGGAGTGCCCCCTAAAGTGGTCAATTACGAGGCCGGGGAAT
GGGCTGAGAATTGCTATAACCTGGAAATCAAGAAACCCGACGGATCAGAGTGTCTGCCAGCC
GCTCCCGATGGGATTCGCGGATTCCCTAGATGCAGATACGTGCACAAGGTCAGCGGCACCGG
GCCATGTGCAGGAGACTTCGCCTTTCATAAAGAAGGCGCCTTCTTTCTGTACGATAGACTGG
CTTCCACCGTGATCTATAGGGGGACCACATTCGCCGAGGGAGTGGTCGCTTTTCTGATTCTG
CCTCAGGCCAAGAAAGACTTCTTTTCTAGTCATCCTCTGCGGGAACCAGTGAACGCTACCGA
GGACCCCAGCAGCGGCTACTATTCCACTACCATCAGATACCAGGCCACAGGATTEGGCACCA
ATGAGACAGAATACCTGTTTGAAGTGGACAACCTGACATATGTCCAGCTGGAGTCTAGGTTC
ACTCCCCAGTTTCTGCTGCAGCTGAATGAAACTATCTATACCAGTGGCAAGCGCTCAAATAC
AACTGGGAAGCTGATTTGGAAAGTGAACCCTGAGATCGATACCACAATTGGCGAATGGGCCT
TTTGGGAGACCAAGAAAAACCTGACACGGAAGATCAGAAGCGAGGAACTGTCCTTCACCGCA
GTGAGTAATAGGGCCAAAAACATTTCAGGCCAGAGCCCAGCACGAACTTCCTCTGACCCCGG
GACCAATACTACCACAGAAGATCACAAGATCATGGCCAGCGAGAACAGTTCAGCTATGGTGC
AGGTCCACTCCCAGGGAAGGGAGGCAGCCGTGTCTCATCTGACTACCCTGGCCACAATCTCT
ACTAGTCCCCAGAGCCCCACAACTAAGCCCGGGCCTGACAATAGCACCCATAACACACCTGT
GTACAAACTGGATATCTCCGAAGCCACCCAGGTCGAGCAGCACCATCGGAGAACAGACAATG
ATTCCACTGCATCTGACACCCCTCCAGCAACCACAGCTGCAGGACCCCCCAAGGCTGAGAAT
ACTAACACCAGCAAAAGCACCGACCTGCTGGACCCCGCAACTACCACATCACCACAGAACCA
CAGCGAGACAGCCGGGAACAATAACACTCACCATCAGGACACCGGAGAGGAATCCGCCAGCT
CCGGCAAGCTGGGGCTGATCACAAATACTATTGCTGGAGTGGCAGGACTGATCACAGGCGGG
AGGEGAACTCGACGAGAAGCTATTGTGAACGCACAGCCCAAATGCAATCCTAACCTGCACTA
TTGGACTACCCAGGACGAGGGAGCAGCTATCGGACTGGCATGGATTCCATACTTTGGGCCCG
CAGCCGAAGGAATCTATACCGAGGGCCTGATGCATAATCAGGATGGACTGATCTGTGGCCTG
CGGCAGCTGGCTAACGAAACAACTCAGGCACTGCAGCTGTTCCTGCGAGCTACCACAGAGCT
GCGGACCTTTAGCATECTGAATCGCAAGGCAATTGACTTCCTGCTGCAGCGATGGGGAGGCA
CATOCCACATCCTGGGACCAGACTGCTGTATTGAGCCTCATGATTGGACAAAGAACATCACT
GACAAAATTGATCAGATCATTCACGACTTCGTGGATAAAACACTGCCAGATCAGGGGGACAA
TGATAACTGGTGGACTGGATGGAGACAGTGGATTCCCGCCGGCATTGGCGTCACCGGCGTCA
TTATTGCCGTCATTGCTCTGTTCTGTATTTGTAAGTTCGTGTTCTGATAA
Protein sequence of Zaire ebolavirus glycoprotein consensus
(SEQ ID NO: 51)
MGVTGILQLPRDRFKRTSFFLWVIILFQRTFSIPLGVIHNSTLQVSDVDKLVCRDKLSSTNQ
LRSVGLNLEGNGVATDVPSATKRWGFRSGVPPKVVNYEAGEWAENCYNLEIKKPDGSECLPA
APDGIRGFPRCRYVHKVSGTGPCAGDFAFHKEGAFFLYDRLASTVIYRGTTFAEGVVAFLIL
PQAKKDFFSSHPLREPVNATEDPSSGYYSTTIRYQATGXGTNETEYLFEVDNLTYVQLESRF
TPQFLLQLNETIYTSGKRSNTIGKLIWKVNPEIDTTIGEWAFWETKKNLTRKIRSEELSFTA
VSNRAKNISGQSPARTSSDPGTNTTTEDHKIMASENSSAMVQVHSQGREAAVSHLTTLATIS
TSPQSPTTKPGPDNSTHNTPVYKLDISEATQVEQHHRRTDNDSTASDTPPATTAAGPPKAEN
TNTSKSTDLLDPATTTSPQNHSETAGNNNTHHQDTGEESASSGKLGLITNTIAGVAGLITGG
RXTRREAIVNAQPKCNPNLHYWTTQDEGAAIGLAWIPYFGPAAEGIYTEGLMHNQDGLICGL
RQLANETTQALQLFLRATTELRTFSXLNRKAIDFLLQRWGGTXHILGPDCCIEPHDWTKNIT
DKIDQIIHDFVDKTLPDQGDNDNWWTGWRQWIPAGIGVTGVIIAVIALFCICKFVF
Sudan Ebolavirus Glycoprotein consensus
DNA sequence
(SEQ ID NO: 52)
ATGGAGGGACTGTCACTGCTGCAGCTGCCTAGAGATAAGTTCAGGAAAAGCTCCTTCTTTGT
GTGGGTCATCATTCTGTTCCAGAAGGCCTTTTCAATGCCCCTGGGCGTGGTCACTAATAGCA
CCCTGGAAGTGACAGAGATCGATCAGCTGGTCTGTAAGGACCACCTGGCTTCAACTGATCAG
CTGAAAAGCGTGGGGOTGAACCTGGAGGGATCAGGCGTCAGCACTGATATTCCTTCTGCAAC
CAAGAGATGGGGATTTCGCAGCGGAGTGCCCCCTAAAGTGGTCTCCTACGAAGCAGGGGAGT
GGGCCGAAAATTGCTATAACCTGGAGATCAAGAAACCAGATGGCAGCGAATGTCTGCCACCC
CCTCCAGACGGGGTGCGCGGATTCCCCAGATGCAGATACGTCCACAAGGOCCAGGGGACCGG
ACCTTGTCCAGGAGACTATGCCTTTCATAAAGATGGCGCTTTCTTTCTGTACGACCGCCTGG
CTAGTACAGTGATETATCGAGGCGTCAATTTCGCCGAGGGCGTGATCGCTTTTCTGATTCTG
GCAAAGCCAAAAGAAACCTTCCTGCAGAGCCCTCCCATTAGGGAGGCCGTGAACTACACAGA
AAACACTTCTAGTTACTACGCTACATCCTACCTGGAGTATGAAATCGAGAACTTTGGCGCTC
AGCACTCTACCACACTGTTCAAGATTAACAATAACACOTTTGTGCTGCTGGATCGCCCTCAT
ACACCACAGTTCCTGTTTCAGCTGAACGACACTATCCACCTGCATCAGCAGCTGAGCAATAC
TACCGGAAAACTGATTTGGACACTGGACGCTAATATCAACGCAGATATTGGCGAGTGGGCCT
TCTGGGAAAATAAGAAAAACCTGTCCGAGCAGCTGCGGGGAGAGGAACTGAGCTTTGAAACA
CTGTCCCTGAATGAAACTGAGGACGATGACGCCACCTCAAGCCGAACAACTAAGGGCCGGAT
CTCTGATCGGGCTACCAGAAAGTACAGTGATCTGGTGCCAAAAGACTCTOCCGGCATGGTGA
GTCTGCACGTCCCTGAAGGGGAGACCACACTGCCATCCCAGAACTCTACTGAGGGCCGGAGA
GTGGACGTCAATACCCAGGAGACTATCACCGAAACTACCGCAACAATCATTGGCACTAACGG
GAATAACATGCAGATCAGCACCATTGGCACAGGGCTGTCCTCTAGTCAGATTCTGTCAAGCT
CCCCTACCATGGCCCCCTCCCCTGAGACACAGACTTCTACAACTTATACACCCAAGCTGCCT
GTGATGACCACAGAGGAACCCACTACCCCACCCAGAAACAGTCCTGGGTCAACAACTGAGGC
ACCCACCCTGACCACACCTGAAAATATCACTACCGCCGTGAAAACAGTCCTGCCTCAGGAGT
CTACTAGTAACGGACTGATCACCAGCACAGTGACTGGAATTCTGGGCAGTCTGGGGCTGCGC
AAGCGATCAAGGCGCCAAGTGAATACTCGGGCTACCGGCAAATGCAATCCAAACCTGCACTA
CTGGACCGCACAGGAGCAGCATAACGCCGCTGGGATCGCTTGGATTCCTTACTTCGGACCAG
GCGCAGAGGGGATCTATACCGAAGGACTGATGCATAATCAGAACGCCCTGGTGTGTGGCCTG
AGACAGCTGGCAAATGAGACAACTCAGGCCCTGCAGCTGTTCCTGAGAGCAACCACAGAACT
GAGGACCTATACAATCCTGAACCGGAAGGCCATTGATTTTCTGCTGCGACGATGGGGCGGGA
CCTGCAGAATCCTGGGACCAGACTGCTGTATTGAGCCCCACGATTGGACCAAGAACATCACA
GACAAGATCAACCAGATCATTCATGATTTCATCGACAACCCACTGCCCAATCAGGACAACGA
TGACAATTGGTGGACCGGATGGCGACAGTGGATTCCCGCAGGAATTGGAATCACCGGAATTA
TTATTGCCATTATTGCTCTGCTGTGTGTCTGTAAGCTGCTGTGTTGATAA
Protein sequence
(SEQ ID NO: 53)
MEGLSLLQLPRDKFRKSSFFVWVIILFQKAFSMPLGVVTNSTLEVTEIDQLVCKDHLASTDQ
LKSVGXNLEGSGVSTDIPSATKRWGFRSGVPPKVVSYEAGEWAENCYNLEIKKPDGSECLPP
PPDGVRGFPRCRYVHKXQGTGPCPGDYAFHKDGAFFLYDRLASTVXYRGVNFAEGVIAFLIL
AKPKETFLQSPPIREAVNYTENTSSYYATSYLEYEIENFGAQHSTTLFKINNNXFVLLDRPH
TPQFLFQLNDTIHLHQQLSNTTGKLIWTLDANINADIGEWAFWENKKNLSEQLRGEELSFET
LSLNETEDDDATSSRTTKGRISDRATRKYSDLVPKDSXGMVSLHVPEGETTLPSQNSTEGRR
VDVNTQETITETTATIIGTNGNNMQISTIGTGLSSSQILSSSPTMAPSPETQTSTTYTPKLP
VMTTEEPTTPPRNSPGSTTEAPTLTTPENITTAVKTVLPQESTSNGLITSTVTGILGSLGLR
KRSRRQVNTRATGKCNPNLHYWTAQEQHNAAGIAWIPYFGPGAEGIYTEGLMHNQNALVCGL
RQLANETTQALQLFLRATTELRTYTILNRKAIDFLLRRWGGTCRILGPDCCIEPHDWTKNIT
DKINQIIHDFIDNPLPNQDNDDNWWTGWRQWIPAGIGITGIITATIALLCVCKLLC
Marburgvirus glycoprotein consensus
DNA sequence
(SEQ ID NO: 54)
ATGAAAACCACTTGTCTGCTPATCTCACTGATTCTGATTCAGGGCGTCAAAACACTGCCCAT
TCTGGAAATTGCCTCTAACATCCAGCCACAGAACGTGGACTCCGTCTGTTCTGGGACCCTGC
AGAAGACAGAGGATGTGCACCTGATGGGCTTCACCCTGAGCGGGCAGAAGGTCGCAGACTCA
CCCCTGGAAGCCAGCAAACGATGGGCATTTCGGGCCGGAGTGCCCCCTAAGAACGTCGAGTA
CACCGAAGGCGAGGAAGCCAAAACATGCTATAATATCTCCGTGACTGATCCTAGTGGCAAGT
CACTGCTGCTGGACCCACCCACCAACATTAGGGATTACCCTAAGTGTAAAACAATCCACCAT
ATTCAGGGCCAGAATCCACACGCTCAGGGGATCGCACTGCATCTGTGGGGAGCCTTCTTTCT
GTACGACAGGATTGCTAGCACCACAATGTATCGCGGGAAAGTGTTCACCGAGGGAAACATCG
CCGCTATGATTGTGAATAAGACAGTCCACAAAATGATCTTTTCTCGCCAGGGCCAGGGGTAC
CGACATATGAACCTGACCAGTACAAATAAGTATTGGACCAGCTCCAACGGCACTCAGACCAA
TGACACTGGGTGCTTCGGAACCCTGCAGGAGTACAACAGTACTAAAAATCAGACCTGTGCTC
CATCAAAGAAACCACTGCCACTGCCTACCGCACACCCAGAGGTGAAGCTGACAAGTACTTCA
ACCGACGCCACAAAACTGAACACTACCGACCCCAATAGTGACGATGAAGATCTGACAACTAG
CGGATCCGGCTCTGGGGAGCAGGAACCTTATACCACATCCGATGCAGCCACCAAGCAGGGCC
TGTCTAGTACAATGCCTCCAACTCCATCTCCCCAGCCTAGTACTCCCCAGCAGGGCGGGAAC
AATACCAACCATTCCCAGGGCGTGGTCACAGAGCCAGGGAAGACTAACACTACCGCCCAGCC
CTCTATGCCCCCTCACAATACAACTACCATCTCCACCAACAATACATCTAAACATAACCTGA
GCACACCTTCCGTGCCAATCCAGAACGCTACTAACTACAACACTCAGTCTACCGCACCCGAG
AATGAACAGACTTCTGCCCCTAGTAAGACAACTCTGCTGCCCACCGAGAACCCTACCACAGC
CAAGTCAACAAATAGCACTAAATCCCCTACTACCACAGTGCCAAACACTACCAATAAGTACA
GTACCTCACCAAGCCCCACCCCTAACTCCACAGCACAGCACCTGGTCTATTTCCGGAGAAAA
AGAAATATCCTGTGGAGGGAGGGCGACATGTTCCCTTTTCTGGATGGGCTGATCAACGCTCC
AATTGACTTCGATCCAGTGCCCAATACAAAGACTATCTTTGACGAATCAAGCTCCTCTGGCG
CCTCTGCTGAGGAAGATCAGCACGCCTCACCCAACATTAGCCTGACACTGTCCTACTTTCCT
AAAGTGAACGAGAATACTGCCCATAGCGGGGAGAACGAAAATGACTGCGATGCTGAGCTGCG
GATCTGGAGCGTCCAGGAAGACGATCTGGCTGCAGGACTGTCCTGGATCCCATTCTTTGGAC
CCGGCATTGAGGGACTGTATACCGCCGGCCTGATTAAGAACCAGAACAACCTGGTGTGCAGA
CTGAGGCGCCTGGCCAATCAGACCGCTAAATCACTGGAACTGCTGCTGCGGGTCACAACTGA
GGAAAGAACATTCAGCCTGATCAACCGACATGCTATTGACTTTCTGCTGGCACGCTGGGGAG
GCACCTGCAAGGTGCTGGGACCAGACTGCTGTATCGGCATTGAGGATCTGTCTCGCAATATC
AGTGAACAGATCGACCAGATTAAGAAAGATGAGCAGAAGGAAGGAACCGGATGGGGACTGGG
CGGCAAGTGGTGGACCAGCGATTGGGGCGTGCTGACAAACCTGGGAATCCTGCTGCTGCTGT
CCATCGCCGTCCTGATTGCTCTGTCCTGTATTTGTCGGATTTTCACTAAGTATATT
GGGTGATAA
Protein sequence
(SEQ ID NO: 55)
MKTTCLXISLILIQGVKTLPILEIASNIQPQNVDSVCSGTLQKTEDVHLMGFTLSGQKVADS
PLEASKRWAFRAGVPPKNVEYTEGEEAKTCYNISVTDPSGKSLLLDPPTNIRDYPKCKTIHH
IQGQNPHAQGIALHLWGAFFLYDRIASTTMYRGKVFTEGNIAAMIVNKTVHKMIFSRQGQGY
RHMNLTSTNKYWTXSNGTQTNDTGCFGTLQEYNSTKNQTCAPSKKPLPLPTAHPEVKLTSTS
TDATKLNTTDPNSDDEDLTTSGSGSGEQEPYTTSDAATKQGLSSTMPPTPSPQPSTPQQGGN
NTNHSQGVVTEPGKTNTTAQPSMPPHNTTTISTNNTSKHNLSTPSVPIQNATNYNTQSTAPE
NEQTSAPSKTTLLPTENPTTAKSTNSTKSPTTTVPNTTNKYSTSPSPTPNSTAQHLVYFRRK
RNILWREGDMFPFLDGLINAPIDFDPVPNTKTIFDESSSSGASAEEDQHASPNISLTLSYFP
KVNENTAHSGENENDCDAELRIWSVQEDDLAAGLSWIPFFGPGIEGLYTAGLIKNQNNLVCR
LRRLANQTAKSLELLLRVTTEERTFSLINRHAIDFLLARWGGTCKVLGPDCCIGIEDLSRNI
SEQIDQIKKDEQKEGTGWGLGGKWWTSDWGVLTNLGILLLLSIAVLIALSCICRIFTKYIG

5. Regulatory Sequences

In some embodiments, the expressible nucleic acid sequence can be operably linked to one or a plurality of regulatory sequences.

B. Nucleic Acid Molecule

In one aspect, the present disclosure also relates to a nucleic acid molecule that comprises any of the disclosed expressible nucleic acid sequences. For example, the nucleic acid molecule can be a plasmid. Provided herein is a vector or plasmid that is capable of expressing a at least a monomer of a self-assembling nanoparticle and a viral antigen construct or constructs in the cell of a mammal in a quantity effective to elicit an immune response in the mammal. The vector may comprise heterologous nucleic acid encoding the one or more viral antigens (such as HIV-1 antigens). The vector may be a plasmid. The plasmid may be useful for transfecting cells with nucleic acid encoding a viral antigen, which the transformed host cell is cultured and maintained under conditions wherein expression of the viral antigen takes place and wherein the structure of the nanoparticle with the antigen elicits an immune response of a magnitude greater than and/or more therapeutically effective than the immune response elicited by the antigen alone. The plasmid may further comprise an initiation codon, which may be upstream of the expressible sequence, and a stop codon, which may be downstream of the coding sequence. The initiation and termination codon may be in frame with the expressible sequence.

The plasmid may also comprise a promoter that is operably linked to the coding sequence The promoter operably linked to the coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, or human metalothionein. The promoter may also be a tissue specific promoter, such as a muscle or skin specific promoter, natural or synthetic. Examples of such promoters are described in US patent application publication no. US20040175727, the contents of which are incorporated herein in its entirety. The plasmid may also comprise a polyadenylation signal, which may be downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, Calif.).

The plasmid may also comprise an enhancer upstream of the coding sequence. The enhancer may be human actin, human myosin, human hemoglobin, human muscle creatine or a viral enhancer such as one from CMV, FMDV, RSV or EBV. Polynucleotide function enhancers are described in U.S. Pat. Nos. 5,593,972, 5,962,428, and WO94/016737, the contents of each are fully incorporated by reference. The plasmid may also comprise a mammalian origin of replication in order to maintain the plasmid extrachromosomally and produce multiple copies of the plasmid in a cell. The plasmid may be pVAX1, pCEP4 or pREP4 from ThermoFisher Scientific (San Diego, Calif.), which may comprise the Epstein Barr virus origin of replication and nuclear antigen EBNA-1 coding region, which may produce high copy episomal replication without integration. The vector can be pVAX1 or a pVax1 variant with changes such as the variant plasmid described herein. The variant pVax1 plasmid is a 2998 basepair variant of the backbone vector plasmid pVAX1 (Invitrogen, Carlsbad Calif.). The CMV promoter is located at bases 137-724. The T7 promoter/priming site is at bases 664-683. Multiple cloning sites are at bases 696-811. Bovine GH polyadenylation signal is at bases 829-1053. The Kanamycin resistance gene is at bases 1226-2020. The pUC origin is at bases 2320-2993. The vaccine may comprise the consensus antigens and plasmids at quantities of from about 1 nanogram to 100 milligrams; about 1 microgram to about 10 milligrams; or preferably about 0.1 microgram to about 10 milligrams; or more preferably about 1 milligram to about 2 milligram. In some embodiments, pharmaceutical compositions according to the present disclosure comprise from about 1 nanogram to about 1000 micrograms of DNA. The nucleic acid sequence for the pVAX1 backbone sequence is as follows:

(SEQ ID NO: 56)
GACTCTTCGCGATGTACGGGCCAGATATACGCGTTGACATTGATTATTGACTAGTTATTAAT
AGTAATCAATTACGGGGTCATTAGTTCATAGCCCATATATGGAGTTCCGCGTTACATAACTT
ACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGACTATTTAC
GGTAAACTGCCCACTTGGCAGTACATCAAGTGTATCATATGCCAAGTACGCCCCCTATTGAC
GTCAATGACGGTAAATGGCCCGCCTGGCATTATGCCCAGTACATGACCTTATGGGACTTTCC
TACTTGGCAGTACATCTACGTATTAGTCATCGCTATTACCATGGTGATGCGGTTTTGGCAGT
ACATCAATGGGCGTGGATAGCGGTTTGACTCACGGGGATTTCCAAGTCTCCACCCCATTGAC
GTCAATGGGAGTTTGTTTTGGCACCAAAATCAACGGGACTTTCCAAAATGTCGTAACAACTC
CGCCCCATTGACGCAAATGGGCGGTAGGCGTGTACGGTGGGAGGTCTATATAAGCAGAGCTC
TCTGGCTAACTAGAGAACCCACTGCTTACTGGCTTATCGAAATTAATACGACTCACTATAGG
GAGACCCAAGCTGGCTAGCGTTTAAACTTAAGCTTGGTACCGAGCTCGGATCCACTAGTCCA
GTGTGGTGGAATTCTGCAGATATCCAGCACAGTGGCGGCCGCTCGAGTCTAGAGGGCCCGTT
TAAACCCGCTGATCAGCCTCGACTGTGCCTTCTAGTTGCCAGCCATCTGTTGTTTGCCCCTC
CCCCGTGCCTTCCTTGACCCTGGAAGGTGCCACTCCCACTGTCCTTTCCTAATAAAATGAGG
AAATTGCATCGCATTGTCTGAGTAGGTGTCATTCTATTCTGGGGGGTGGGGTGGGGCAGGAC
AGCAAGGGGGAGGATTGGGAAGACAATAGCAGGCATGCTGGGGATGCGGTGGGCTCTATGGC
TTCTACTGGGCGGTTTTATGGACAGCAAGCGAACCGGAATTGCCAGCTGGGGCGCCCTCTGG
TAAGGTTGGGAAGCCCTGCAAAGTAAACTGGATGGCTTTCTCGCCGCCAAGGATCTGATGGC
GCAGGGGATCAAGCTCTGATCAAGAGACAGGATGAGGATCGTTTCGCATGATTGAACAAGAT
GGATTGCACGCAGGTTCTCCGGCCGCTTGGGTGGAGAGGCTATTCGGCTATGACTGGGCACA
ACAGACAATCGGCTGCTCTGATGCCGCCGTGTTCCGGCTGTCAGCGCAGGGGCGCCCGGTTC
TTTTTGTCAAGACCGACCTGTCCGGTGCCCTGAATGAACTGCAAGACGAGGCAGCGCGGCTA
TCGTGGCTGGCCACGACGGGCGTTCCTTGCGCAGCTGTGCTCGACGTTGTCACTGAAGCGGG
AAGGGACTGGCTGCTATTGGGCGAAGTGCCGGGGCAGGATCTCCTGTCATCTCACCTTGCTC
CTGCCGAGAAAGTATCCATCATGGCTGATGCAATGCGGCGGCTGCATACGCTTGATCCGGCT
ACCTGCCCATTCGACCACCAAGCGAAACATCGCATCGAGCGAGCACGTACTCGGATGGAAGC
CGGTCTTGTCGATCAGGATGATCTGGACGAAGAGCATCAGGGGCTCGCGCCAGCCGAACTGT
TCGCCAGGCTCAAGGCGAGCATGCCCGACGGCGAGGATCTCGTCGTGACCCATGGCGATGCC
TGCTTGCCGAATATCATGGTGGAAAATGGCCGCTTTTCTGGATTCATCGACTGTGGCCGGCT
GGGTGTGGCGGACCGCTATCAGGACATAGCGTTGGCTACCCGTGATATTGCTGAAGAGCTTG
GCGGCGAATGGGCTGACCGCTTCCTCGTGCTTTACGGTATCGCCGCTCCCGATTCGCAGCGC
ATCGCCTTCTATCGCCTTCTTGACGAGTTCTTCTGAATTATTAACGCTTACAATTTCCTGAT
GCGGTATTTTCTCCTTACGCATCTGTGCGGTATTTCACACCGCATACAGGTGGCACTTTTCG
GGGAAATGTGCGCGGAACCCCTATTTGTTTATTTTTCTAAATACATTCAAATATGTATCCGC
TCATGAGACAATAACCCTGATAAATGCTTCAATAATAGCACGTGCTAAAACTTCATTTTTAA
TTTAAAAGGATCTAGGTGAAGATCCTTTTTGATAATCTCATGACCAAAATCCCTTAACGTGA
GTTTTCGTTCCACTGAGCGTCAGACCCCGTAGAAAAGATCAAAGGATCTTCTTGAGATCCTT
TTTTTCTGCGCGTAATCTGCTGCTTGCAAACAAAAAAACCACCGCTACCAGCGGTGGTTTGT
TTGCCGGATCAAGAGCTACCAACTCTTTTTCCGAAGGTAACTGGCTTCAGCAGAGCGCAGAT
ACCAAATACTGTCCTTCTAGTGTAGCCGTAGTTAGGCCACCACTTCAAGAACTCTGTAGCAC
CGCCTACATACCTCGCTCTGCTAATCCTGTTACCAGTGGCTGCTGCCAGTGGCGATAAGTCG
TGTCTTACCGGGTTGGACTCAAGACGATAGTTACCGGATAAGGCGCAGCGGTCGGGCTGAAC
GGGGGGTTCGTGCACACAGCCCAGCTTGGAGCGAACGACCTACACCGAACTGAGATACCTAC
AGCGTGAGCTATGAGAAAGCGCCACGCTTCCCGAAGGGAGAAAGGCGGACAGGTATCCGGTA
AGCGGCAGGGTCGGAACAGGAGAGCGCACGAGGGAGCTTCCAGGGGGAAACGCCTGGTATCT
TTATAGTCCTGTCGGGTTTCGCCACCTCTGACTTGAGCGTCGATTTTTGTGATGCTCGTCAG
GGGGGCGGAGCCTATGGAAAAACGCCAGCAACGCGGCCTTTTTACGGTTCCTGGGCTTTTGC
TGGCCTTTTGCTCACATGTTCTT

In some embodiments, the disclosure relates to a composition comprising a nucleic acid molecule comprising at least about 70%, 75%, 80%, 85%, 90%, 91%, 92%, 93%, 94%, 95%, 96%, 97%, 99% or 100% sequence identity to SEQ ID NO:56 or a pharmaceutically acceptable salt thereof. In some embodiments, the disclosure relates to a composition comprising a nucleic acid molecule that is a pVax variant or pharmaceutically acceptable salt thereof. In some embodiments, the disclosure relates to a composition comprising a nucleic acid molecule comprising at least about 70%, 75%, 80%, 85%, 90%, 91%, 92%, 93%, 94%, 95%, 96%, 97%, 99% or 100% sequence identity to SEQ ID NO:56 or a pharmaceutically acceptable salt thereof and an expressible nucleic acid sequence comprising at least about 70%, 75%, 80%, 85%, 90%, 91%, 92%, 93%, 94%, 95%, 96%, 97%, 99% or 100% sequence identity to SEQ ID NO:5, SEQ ID NO:20, SEQ ID NO:24, SEQ ID NO:28, SEQ ID NO:33, SEQ ID NO:34, SEQ ID NO:37, SEQ ID NO:57, SEQ ID NO:59, SEQ ID NO:61 or SEQ ID NO:63. In some embodiments, the disclosure relates to a composition comprising a nucleic acid molecule comprising at least about 70%, 75%, 80%, 85%, 90%, 91%, 92%, 93%, 94%, 95%, 96%, 97%, 99% or 100% sequence identity to SEQ ID NO:56 or a pharmaceutically acceptable salt thereof and an expressible nucleic acid sequence encoding a polypeptide comprising at least about 70%, 75%, 80%, 85%, 90%, 91%, 92%, 93%, 94%, 95%, 96%, 97%, 99% or 100% sequence identity to SEQ ID NO:10, SEQ ID NO:21, SEQ ID NO:25, SEQ ID NO:30, SEQ ID NO:35, SEQ ID NO:36, SEQ ID NO:38, SEQ ID NO:58, SEQ ID NO:60, SEQ ID NO:62, SEQ ID NO:64, SEQ ID NO:65, SEQ ID NO:66 or SEQ ID NO:67. In some embodiments, the disclosure relates to a composition comprising a nucleic acid molecule comprising at least about 70%, 75%, 80%, 85%, 90%, 91%, 92%, 93%, 94%, 95%, 96%, 97%, 99% or 100% sequence identity to SEQ ID NO:56 or a pharmaceutically acceptable salt thereof and an expressible nucleic acid sequence comprising at least about 70%, 75%, 80%, 85%, 90%, 91%, 92%, 93%, 94%, 95%, 96%, 97%, 99% or 100% sequence identity to SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:10, SEQ ID NO:11 or SEQ ID NO:12. In some embodiments, the disclosure relates to nucleic acid molecules comprising a plasmid comprising a regulatory sequence operably linked one or more expressible nucleic acid sequences, wherein the expressible nucleic acid sequences comprise at least a first nucleic acid sequence that is a self-assembling polypeptide, a second nucleic acid sequence that encodes any one or plurality of viral antigens disclosed herein. In some embodiments, the first and second nucleic acids are linked by a linker disclosed herein. In some embodiments, the first and second nucleic acids are in a 5′ to 3′ orientation and fused to an IgE or IgG linker positioned 5′ of the 5′ end of the first and/or second nucleic acid sequence. In some embodiments, the disclosure relates to a composition comprising a nucleic acid molecule comprising at least about 70%, 75%, 80%, 85%, 90%, 91%, 92%, 93%, 94%, 95%, 96%, 97%, 99% or 100% sequence identity to SEQ ID NO:56 or a pharmaceutically acceptable salt thereof and positioned within a multiple cloning site are one or more expressible nucleic acid sequences.

In some embodiments, the plasmid comprises an expressible nucleic acid sequence comprising at least about 70%, 75%, 80%, 85%, 90%, 91%, 92%, 93%, 94%, 95%, 96%, 97%, 99% or 100% sequence identity to SEQ ID NO:5 or a pharmaceutically acceptable salt thereof.

(SEQ ID NO: 5)
ATGGACTGGACCTGGATTCTGTTCCTGGTGGCCGCCGCCACAAGGGTGCA
CAGCATGCAGATCTACGAAGGAAAACTGACCGCTGAGGGACTGAGGTTCG
GAATTGTCGCAAGCCGCGCGAATCACGCACTGGTGGATAGGCTGGTGGAA
GGCGCTATCGACGCAATTGTCCGGCACGGCGGGAGAGAGGAAGACATCAC
ACTGGTGAGAGTCTGCGGCAGCTGGGAGATTCCCGTGGCAGCTGGAGAAC
TGGCTCGAAAGGAGGACATCGATGCCGTGATCGCTATTGGGGTCCTGTGC
CGAGGAGCAACTCCCAGCTTCGACTACATCGCCTCAGAAGTGAGCAAGGG
GCTGGCTGATCTGTCCCTGGAGCTGAGGAAACCTATCACTTTTGGCGTGA
TTACTGCCGACACCCTGGAACAGGCAATCGAGGCGGCCGGCACCTGCCAT
GGAAACAAAGGCTGGGAAGCAGCCCTGTGCGCTATTGAGATGGCAAATCT
GTTCAAATCTCTGCGAGGAGGCTCCGGAGGATCTGGAGGGAGTGGAGGCT
CAGGAGGAGGCGACACCATCACACTGCCATGCCGCCCTGCACCACCTCCA
CATTGTAGCTCCAACATCACCGGCCTGATTCTGACAAGACAGGGGGGATA
TAGTAACGATAATACCGTGATTTTCAGGCCCTCAGGAGGGGACTGGAGGG
ACATCGCACGATGCCAGATTGCTGGAACAGTGGTCTCTACTCAGCTGTTT
CTGAACGGCAGTCTGGCTGAGGAAGAGGTGGTCATCCGATCTGAAGACTG
GCGGGATAATGCAAAGTCAATTTGTGTGCAGCTGAACACAAGCGTCGAGA
TCAATTGCACTGGCGCAGGGCACTGTAACATTTCTCGGGCCAAATGGAAC
AATACCCTGAAGCAGATCGCCAGTAAACTGAGAGAGCAGTACGGCAATAA
GACAATCATCTTCAAGCCTTCTAGTGGAGGCGACCCAGAGTTCGTGAACC
ATAGCTTTAATTGCGGGGGAGAGTTCTTTTATTGTGATTCCACACAGCTG
TTCAACAGCACTTGGTTTAATTCCACCTGATAA

Thus, in some embodiments, the disclosed compositions can be vectors comprising a DNA backbone with an expressible insert comprising one or more of the disclosed leader sequences, self-assembling polypeptides, linkers and viral antigens.

### C. Polypeptide Sequences

Disclosed are the polypeptide sequences encoded by the disclosed nucleic acid sequences. In some embodiments, the disclosure relates to compositions comprising polypeptide sequences encoded by the leader sequence, self-assembling polypeptide encoded by a nucleotide sequence, polypeptide sequences encoded by the linker, and viral antigens encoded by a nucleotide sequence. The disclosure also relates to cells expressing one or more polypeptides disclosed in the application.

In some embodiments, the polypeptide encoded by the leader sequence can be the IgE amino acid sequence MDWTWILFLVAAATRVHS (SEQ ID NO:6) encoded by SEQ ID NO:1, or a functional fragment thereof comprising at least about 70%, 75%, 80%, 85%, 90%, 91%, 92%, 93%, 94%, 95%, 96%, 97% or 99% sequence identity to SEQ ID NO:6. In some embodiments, the polypeptide encoded by the leader sequence comprises the amino acid sequence of SEQ ID NO:40, or a functional fragment thereof comprising at least about 70%, 75%, 80%, 85%, 90%, 91%, 92%, 93%, 94%, 95%, 96%, 97% or 99% sequence identity to SEQ ID NO:40.

In some embodiments, the self-assembling polypeptide can be MQIYEGKLTAEGLRFGIVASRANHALVDRLVEGAIDAIVRHGGREEDITLVRVCGSWEIPVA AGELARKEDIDAVIAIGVLCRGATPSFDYIASEVSKGLADLSLELRKPITFGVITADTLEQA IEAAGTCHGNKGWEAALCAIEMANLFKSLR (SEQ ID NO:7) encoded by SEQ ID NO:2, or a functional fragment thereof comprising at least about 70%, 75%, 80%, 85%, 90%, 91%, 92%, 93%, 94%, 95%, 96%, 97% or 99% sequence identity to SEQ ID NO:7. In some embodiments, the self-assembling polypeptide comprises the amino acid sequence of SEQ ID NO:23, SEQ ID NO:26 or SEQ ID NO:31, or a functional fragment thereof comprising at least about 70%, 75%, 80%, 85%, 90%, 91%, 92%, 93%, 94%, 95%, 96%, 97% or 99% sequence identity to SEQ ID NO:23, SEQ ID NO:26 or SEQ ID NO:31.

In some embodiments, the polypeptide sequences encoded by the linker sequence comprises GGSGGSGGSGGSGGG (SEQ ID NO:8) encoded by SEQ ID NO:3, or a functional fragment thereof comprising at least about 70%, 75%, 80%, 85%, 90%, 91%, 92%, 93%, 94%, 95%, 96%, 97% or 99% sequence identity to SEQ ID NO:3. In some embodiments, the polypeptide sequences encoded by the linker sequence comprises the amino acid sequence of SEQ ID NO:18, SEQ ID NO:22, SEQ ID NO:27 or SEQ ID NO:32, or a functional fragment thereof comprising at least about 70%, 75%, 80%, 85%, 90%, 91%, 92%, 93%, 94%, 95%, 96%, 97% or 99% sequence identity to SEQ ID NO:18, SEQ ID NO:22, SEQ ID NO:27 or SEQ ID NO:32.

In some embodiments, the viral antigen comprises

(SEQ ID NO: 9)
DTITLPCRPAPPPHCSSNITGLILTRQGGYSNDNTVIFRPSGGDWRDIAR
CQTAGTVVSTQLFLNGSLAEEEVVIRSEDWRDNAKSICVQLNTSVEINCT
GAGHCNISRAKWNNTLKQIASKLREQYGNKTIIFKPSSGGDPEFVNHSFN
CGGEFFYCDSTQLFNSTWFNST

encoded by SEQ ID NO: 4, or a functional fragment thereof comprising at least about 70%, 75%, 80%, 85%, 90%, 91%, 92%, 93%, 94%, 95%, 96%, 97% or 99% sequence identity to SEQ ID NO:4. In some embodiments, the viral antigen comprises the amino acid sequence of SEQ ID NO:45, SEQ ID NO:47, SEQ ID NO:49, SEQ ID NO:51, SEQ ID NO:53 or SEQ ID NO:55, or a functional fragment thereof comprising at least about 70%, 75%, 80%, 85%, 90%, 91%, 92%, 93%, 94%, 95%, 96%, 97% or 99% sequence identity to SEQ ID NO:45, SEQ ID NO:47, SEQ ID NO:49, SEQ ID NO:51, SEQ ID NO:53 or SEQ ID NO:55.

In some embodiments, the nucleic acid molecule of the present disclosure encodes a polypeptide comprising

(SEQ ID NO: 10)
MDWTWILFLVAAATRVHSMQIYEGKLTAEGLRFGIVASRANHALVDRLVE
GAIDAIVRHGGREEDITLVRVCGSWEIPVAAGELARKEDIDAVIAIGVLC
RGATPSFDYIASEVSKGLADLSLELRKPITFGVITADTLEQAIEAAGTCH
GNKGWEAALCAIEMANLFKSLRGGSGGSGGSGGSGGGDTITLPCRPAPPP
HCSSNITGLILTRQGGYSNDNTVIFRPSGGDWRDIARCQIAGTVVSTQLF
LNGSLAEEEVVIRSEDWRDNAKSICVQLNTSVEINCTGAGHCNISRAKWN
NTLKQIASKLREQYGNKTIIFKPSSGGDPEFVNHSFNCGGEFFYCDSTQL
FNSTWFNST

encoded by SEQ ID NO:5, or a functional fragment thereof comprising at least about 70%, 75%, 80%, 85%, 90%, 91%, 92%, 93%, 94%, 95%, 96%, 97% or 99% sequence identity to SEQ ID NO:10. In some embodiments, the nucleic acid molecule of the present disclosure encodes a polypeptide comprising the amino acid sequence of SEQ ID NO:21, SEQ ID NO:25, SEQ ID NO:30, SEQ ID NO:35, SEQ ID NO:36, SEQ ID NO:38, SEQ ID NO:58, SEQ ID NO:60, SEQ ID NO:62, SEQ ID NO:64, SEQ ID NO:65, SEQ ID NO:66 or SEQ ID NO:67, or a functional fragment thereof comprising at least about 70%, 75%, 80%, 85%, 90%, 91%, 92%, 93%, 94%, 95%, 96%, 97% or 99% sequence identity to SEQ ID NO:21, SEQ ID NO:25, SEQ ID NO:30, SEQ ID NO:35, SEQ ID NO:36, SEQ ID NO:38, SEQ ID NO:58, SEQ ID NO:60, SEQ ID NO:62, SEQ ID NO:64, SEQ ID NO:65, SEQ ID NO:66 or SEQ ID NO:67.

Also disclosed is the polypeptide comprising the IgE leader sequence and a gp120 variant viral antigen comprising the sequence

(SEQ ID NO: 12)
MDWTWILFLVAAATRVHSDTITLPCRPAPPPHCSSNITGLILTRQGGYSN
DNTVIFRPSGGDWRDIARCQIAGTVVSTQLFLNGSLAEEEVVIRSEDWRD
NAKSICVQLNTSVEINCTGAGHCNISRAKWNNTLKQIASKLREQYGNKTI
IFKPSSGGDPEFVNHSFNCGGEFFYCDSTQLFNSTWFNST

and encoded for by the nucleic acid sequence of SEQ ID NO: 11

Recitation of Sequences.

IgE-GLT1-3BVE
Entire Expressible Nucleic Acid Sequence expressing 3BVE
(SEQ ID NO: 20)
ATGGACTGGACCTGGATTCTGTTCCTGGTGGCCGCCGCCACAAGGGTGCACAGCGACACCAT
CACACTGCCATGCCGCCCTGCACCACCTCCACATTGTAGCTCCAACATCACCGGCCTGATTC
TGACAAGACAGGGGGGATATAGTAACGATAATACCGTGATTTTCAGGCCCTCAGGAGGGGAC
TGGAGGGACATCGCACGATGCCAGATTGCTGGAACAGTGGTCTCTACTCAGCTGTTTCTGAA
CGGCAGTCTGGCTGAGGAAGAGGTGGTCATCCGATCTGAAGACTGGCGGGATAATGCAAAGT
CAATTTGTGTGCAGCTGAACACAAGCGTCGAGATCAATTGCACTGGCGCAGGGCACTGTAAC
ATTTCTCGGGCCAAATGGAACAATACCCTGAAGCAGATCGCCAGTAAACTGAGAGAGCAGTA
CGGCAATAAGACAATCATCTTCAAGCCTTCTAGTGGAGGCGACCCAGAGTTCGTGAACCATA
GCTTTAATTGCGGGGGAGAGTTCTTTTATTGTGATTCCACACAGCTGTTCAACAGCACTTGG
TTTAATTCCACCGGCGGAAGCGGCGGAAGCGGCGGGTCTGGGCTGAGTAAGGACATTATCAA
GCTGCTGAACGAACAGGTGAACAAAGAGATGCAGTCTAGCAACCTGTACATGTCCATGAGCT
CCTGGTGCTATACCCACTCTCTGGACGGAGCAGGCCTGTTCCTGTTTGATCACGCCGCCGAG
GAGTACGAGCACGCCAAGAAGCTGATCATCTTCCTGAATGAGAACAATGTGCCCGTGCAGCT
GACCTCTATCAGCGCCCCTGAGCACAAGTTCGAGGGCCTGACACAGATCTTTCAGAAGGCCT
ACGAGCACGAGCAGCACATCTCCGAGTCTATCAACAATATCGTGGACCACGCCATCAAGTCC
AAGGATCACGCCACATTCAACTTTCTGCAGTGGTACGTGGCCGAGCAGCACGAGGAGGAGGT
GCTGTTTAAGGACATCCTGGATAAGATCGAGCTGATCGGCAATGAGAACCACGGGCTGTACC
TGGCAGATCAGTATGTCAAGGGCATCGCTAAGTCAAGGAAAAGCTGATAA
Linker sequence
(SEQ ID NO: 17)
GGCGGAAGCG GCGGAAGCGGCGGGTCT
3BVE
(Forms 24 mer - SEQ ID NO: 13)
GGGCTGAGTAAGGACATTATCAAGCTGCTGAACGAACAGGTGAACAAAGAGATGCAGTCTAG
CAACCTGTACATGTCCATGAGCTCCTGGTGCTATACCCACTCTCTGGACGGAGCAGGCCTGT
TCCTGTTTGATCACGCCGCCGAGGAGTACGAGCACGCCAAGAAGCTGATCATCTTCCTGAAT
GAGAACAATGTGCCCGTGCAGCTGACCTCTATCAGCGCCCCTGAGCACAAGTTCGAGGGCCT
GACACAGATCTTTCAGAAGGCCTACGAGCACGAGCAGCACATCTCCGAGTCTATCAACAATA
TCGTGGACCACGCCATCAAGTCCAAGGATCACGCCACATTCAACTTTCTGCAGTGGTACGTG
GCCGAGCAGCACGAGGAGGAGGTGCTGTTTAAGGACATCCTGGATAAGATCGAGCTGATCGG
CAATGAGAACCACGGGCTGTACCTGGCAGATCAGTATGTCAAGGGCATCGCTAAGTCAAGGA
AAAGC
Entire Expressed amino acid sequence
(SEQ ID NO: 21)
MDWTWILFLVAAATRVHSDTITLPCRPAPPPHCSSNITGLILTRQGGYSNDNTVIFRPSGGD
WRDIARCQIAGTVVSTQLFLNGSLAEEEVVIRSEDWRDNAKSICVQLNTSVEINCTGAGHCN
ISRAKWNNTLKQIASKLREQYGNKTIIFKPSSGGDPEFVNHSFNCGGEFFYCDSTQLFNSTW
FNSTGGSGGSGGSGLSKDIIKLLNEQVNKEMQSSNLYMSMSSWCYTHSLDGAGLFLFDHAAE
EYEHAKKLIIFLNENNVPVQLTSISAPEHKFEGLTQIFQKAYEHEQHISESINNIVDHAIKS
KDHATFNFLQWYVAEQHEEEVLFKDILDKIELIGNENHGLYLADQYVKGIAKSRKS
Linker
(SEQ ID NO: 22)
GGSGGSGGS
3BVE Scaffold
(SEQ ID NO: 23)
GLSKDIIKLLNEQVNKEMQSSNLYMSMSSWCYTHSLDGAGLFLFDHAAEEYEHAKKLIIFLN
ENNVPVQLTSISAPEHKFEGLTQIFQKAYEHEQHISESINNIVDHAIKSKDHATFNFLQWYV
AEQHEEEVLFKDILDKIELIGNENHGLYLADQYVKGIAKSRKS
IgE-GLT1-I3
Entire Expressible Nucleic Acid Sequence expressing 13
(SEQ ID NO: 24)
ATGGACTGGACCTGGATTCTGTTCCTGGTGGCCGCCGCCACAAGGGTGCACAGCGACACCAT
CACACTGCCATGCCGCCCTGCACCACCTCCACATTGTAGCTCCAACATCACCGGCCTGATTC
TGACAAGACAGGGGGGATATAGTAACGATAATACCGTGATTTTCAGGCCCTCAGGAGGGGAC
TGGAGGGACATCGCACGATGCCAGATTGCTGGAACAGTGGTCTCTACTCAGCTGTTTCTGAA
CGGCAGTCTGGCTGAGGAAGAGGTGGTCATCCGATCTGAAGACTGGCGGGATAATGCAAAGT
CAATTTGTGTGCAGCTGAACACAAGCGTCGAGATCAATTGCACTGGCGCAGGGCACTGTAAC
ATTTCTCGGGCCAAATGGAACAATACCCTGAAGCAGATCGCCAGTAAACTGAGAGAGCAGTA
CGGCAATAAGACAATCATCTTCAAGCCTTCTAGTGGAGGCGACCCAGAGTTCGTGAACCATA
GCTTTAATTGCGGGGGAGAGTTCTTTTATTGTGATTCCACACAGCTGTTCAACAGCACTTGG
TTTAATTCCACCGGCGGCAGCGGCGGCAGCGGCGGGAGCGGAGGAAGTGAGAAAGCAGCCAA
AGCAGAGGAAGCAGCACGGAAGATGGAAGAACTGTTCAAGAAGCACAAGATCGTGGCCGTGC
TGAGGGCCAACTCCGTGGAGGAGGCCAAGAAGAAGGCCCTGGCCGTGTTCCTGGGCGGCGTG
CACCTGATCGAGATCACCTTTACAGTGCCCGACGCCGATACCGTGATCAAGGAGCTGTCTTT
CCTGAAGGAGATGGGAGCAATCATCGGAGCAGGAACCGTGACAAGCGTGGAGCAGTGCAGAA
AGGCCGTGGAGAGCGGCGCCGAGTTTATCGTGTCCCCTCACCTGGACGAGGAGATCTCTCAG
TTCTGTAAGGAGAAGGGCGTGTTTTACATGCCAGGCGTGATGACCCCCACAGAGCTGGTGAA
GGCCATGAAGCTGGGCCACACAATCCTGAAGCTGTTCCCTGGCGAGGTGGTGGGCCCACAGT
TTGTGAAGGCCATGAAGGGCCCCTTCCCTAATGTGAAGTTTGTGCCCACCGGCGGCGTGAAC
CTGGATAACGTGTGCGAGTGGTTCAAGGCAGGCGTGCTGGCAGTGGGCGTGGGCAGCGCCCT
GGTGAAGGGCACACCCGTGGAAGTCGCTGAGAAGGCAAAGGCATTCGTGGAAAAGATTAGGG
GGTGTACTGAGTGATAA
Linker
(SEQ ID NO: 19)
GGCGGCAGCGGCGGCAGCGGCGGGAGCGGAGGAAGT
I3 Scaffold
(SEQ ID NO: 15)
GAGAAAGCAGCCAAAGCAGAGGAAGCAGCACGGAAGATGGAAGAACTGTTCAAGAAGCACAA
GATCGTGGCCGTGCTGAGGGCCAACTCCGTGGAGGAGGCCAAGAAGAAGGCCCTGGCCGTGT
TCCTGGGCGGCGTGCACCTGATCGAGATCACCTTTACAGTGCCCGACGCCGATACCGTGATC
AAGGAGCTGTCTTTCCTGAAGGAGATGGGAGCAATCATCGGAGCAGGAACCGTGACAAGCGT
GGAGCAGTGCAGAAAGGCCGTGGAGAGCGGCGCCGAGTTTATCGTGTCCCCTCACCTGGACG
AGGAGATCTCTCAGTTCTGTAAGGAGAAGGGCGTGTTTTACATGCCAGGCGTGATGACCCCC
ACAGAGCTGGTGAAGGCCATGAAGCTGGGCCACACAATCCTGAAGCTGTTCCCTGGCGAGGT
GGTGGGCCCACAGTTTGTGAAGGCCATGAAGGGCCCCTTCCCTAATGTGAAGTTTGTGCCCA
CCGGCGGCGTGAACCTGGATAACGTGTGCGAGTGGTTCAAGGCAGGCGTGCTGGCAGTGGGC
GTGGGCAGCGCCCTGGTGAAGGGCACACCCGTGGAAGTCGCTGAGAAGGCAAAGGCATTCGT
GGAAAAGATTAGGGGGTGTACTGAG
Entire Expressed Amino Acid sequence with I3 - linker- Antigen
(SEQ ID NO: 25)
MDWTWILFLVAAATRVHSDTITLPCRPAPPPHCSSNITGLILTRQGGYSNDNTVIFRPSGGD
WRDIARCQTAGTVVSTQLFLNGSLAEEEVVIRSEDWRDNAKSICVQLNTSVEINCTGAGHCN
ISRAKWNNTLKQIASKLREQYGNKTIIFKPSSGGDPEFVNHSFNCGGEFFYCDSTQLFNSTW
FNSTGGSGGSGGSGGSEKAAKAEEAARKMEELFKKHKIVAVLRANSVEEAKKKALAVFLGGV
HLIEITFTVPDADTVIKELSFLKEMGAIIGAGTVTSVEQCRKAVESGAEFIVSPHLDEEISQ
FCKEKGVFYMPGVMTPTELVKAMKLGHTILKLFPGEVVGPQFVKAMKGPFPNVKFVPTGGVN
LDNVCEWFKAGVLAVGVGSALVKGTPVEVAEKAKAFVEKIRGCTE
I3 Scaffold
(SEQ ID NO: 26)
EKAAKAEEAARKMEELFKKHKIVAVLRANSVEEAKKKALAVFLGGVHLIEITFTVPDADTVI
KELSFLKEMGAIIGAGTVTSVEQCRKAVESGAEFIVSPHLDEEISQFCKEKGVFYMPGVMTP
TELVKAMKLGHTILKLFPGEVVGPQFVKAMKGPFPNVKFVPTGGVNLDNVCEWFKAGVLAVG
VGSALVKGTPVEVAEKAKAFVEKIRGCTE
Linker
(SEQ ID NO: 27)
GGSGGSGGSGGS
IgE-GLT1-RBE
Entire Expressible Nucleic Acid Sequence expressing RBE
(SEQ ID NO: 28)
ATGGACTGGACCTGGATTCTGTTCCTGGTGGCCGCCGCCACAAGGGTGCACAGCCTGAGCAT
TGCCCCCACACTGATTAACCGGGACAAACCCTACACCAAAGAGGAACTGATGGAGATTCTGA
GACTGGCTATTATCGCTGAGCTGGACGCCATCAACCTGTACGAGCAGATGGCCCGGTATTCT
GAGGACGAGAATGTGCGCAAGATCCTGCTGGATGTGGCCAGGGAGGAGAAGGCACACGTGGG
AGAGTTCATGGCCCTGCTGCTGAACCTGGACCCCGAGCAGGTGACCGAGCTGAAGGGCGGCT
TTGAGGAGGTGAAGGAGCTGACAGGCATCGAGGCCCACATCAACGACAATAAGAAGGAGGAG
AGCAACGTGGAGTATTTCGAGAAGCTGAGATCCGCCCTGCTGGATGGCGTGAATAAGGGCAG
GAGCCTGCTGAAGCACCTGCCTGTGACCAGGATCGAGGGCCAGAGCTTCAGAGTGGACATCA
TCAAGTTTGAGGATGGCGTGCGCGTGGTGAAGCAGGAGTACAAGCCCATCCCTCTGCTGAAG
AAGAAGTTCTACGTGGGCATCAGGGAGCTGAACGACGGCACCTACGATGTGAGCATCGCCAC
AAAGGCCGGCGAGCTGCTGGTGAAGGACGAGGAGTCCCTGGTCATCCGCGAGATCCTGTCTA
CAGAGGGCATCAAGAAGATGAAGCTGAGCTCCTGGGACAATCCAGAGGAGGCCCTGAACGAT
CTGATGAATGCCCTGCAGGAGGCATCTAACGCAAGCGCCGGACCATTCGGCCTGATCATCAA
TCCCAAGAGATACGCCAAGCTGCTGAAGATCTATGAGAAGTCCGGCAAGATGCTGGTGGAGG
TGCTGAAGGAGATCTTCCGGGGCGGCATCATCGTGACCCTGAACATCGATGAGAACAAAGTG
ATCATCTTTGCCAACACCCCTGCCGTGCTGGACGTGGTGGTGGGACAGGATGTGACACTGCA
GGAGCTGGGACCAGAGGGCGACGATGTGGCCTTTCTGGTGTCCGAGGCCATCGGCATCAGGA
TCAAGAATCCAGAGGCAATCGTGGTGCTGGAGGGCGGCTCTGGCGGAAGTGGCGGAAGTGGG
GGAAGTGGAGGCGGCGGAAGCGGGGGAGGCAGCGGGGGAGGGGACACCATCACACTGCCATG
CCGCCCTGCACCACCTCCACATTGTAGCTCCAACATCACCGGCCTGATTCTGACAAGACAGG
GGGGATATAGTAACGATAATACCGTGATTTTCAGGCCCTCAGGAGGGGACTGGAGGGACATC
GCACGATGCCAGATTGCTGGAACAGTGGTCTCTACTCAGCTGTTTCTGAACGGCAGTCTGGC
TGAGGAAGAGGTGGTCATCCGATCTGAAGACTGGCGGGATAATGCAAAGTCAATTTGTGTGC
AGCTGAACACAAGCGTCGAGATCAATTGCACTGGCGCAGGGCACTGTAACATTTCTCGGGCC
AAATGGAACAATACCCTGAAGCAGATCGCCAGTAAACTGAGAGAGCAGTACGGCAATAAGAC
AATCATCTTCAAGCCTTCTAGTGGAGGCGACCCAGAGTTCGTGAACCATAGCTTTAATTGCG
GGGGAGAGTTCTTTTATTGTGATTCCACACAGCTGTTCAACAGCACTTGGTTTAATTCCACC
RBE Scaffold
(SEQ ID NO: 14)
CTGAGCATTGCCCCCACACTGATTAACCGGGACAAACCCTACACCAAAGAGGAACTGATGGA
GATTCTGAGACTGGCTATTATCGCTGAGCTGGACGCCATCAACCTGTACGAGCAGATGGCCC
GGTATTCTGAGGACGAGAATGTGCGCAAGATCCTGCTGGATGTGGCCAGGGAGGAGAAGGCA
CACGTGGGAGAGTTCATGGCCCTGCTGCTGAACCTGGACCCCGAGCAGGTGACCGAGCTGAA
GGGCGGCTTTGAGGAGGTGAAGGAGCTGACAGGCATCGAGGCCCACATCAACGACAATAAGA
AGGAGGAGAGCAACGTGGAGTATTTCGAGAAGCTGAGATCCGCCCTGCTGGATGGCGTGAAT
AAGGGCAGGAGCCTGCTGAAGCACCTGCCTGTGACCAGGATCGAGGGCCAGAGCTTCAGAGT
GGACATCATCAAGTTTGAGGATGGCGTGCGCGTGGTGAAGCAGGAGTACAAGCCCATCCCTC
TGCTGAAGAAGAAGTTCTACGTGGGCATCAGGGAGCTGAACGACGGCACCTACGATGTGAGC
ATCGCCACAAAGGCCGGCGAGCTGCTGGTGAAGGACGAGGAGTCCCTGGTCATCCGCGAGAT
CCTGTCTACAGAGGGCATCAAGAAGATGAAGCTGAGCTCCTGGGACAATCCAGAGGAGGCCC
TGAACGATCTGATGAATGCCCTGCAGGAGGCATCTAACGCAAGCGCCGGACCATTCGGCCTG
ATCATCAATCCCAAGAGATACGCCAAGCTGCTGAAGATCTATGAGAAGTCCGGCAAGATGCT
GGTGGAGGTGCTGAAGGAGATCTTCCGGGGCGGCATCATCGTGACCCTGAACATCGATGAGA
ACAAAGTGATCATCTTTGCCAACACCCCTGCCGTGCTGGACGTGGTGGTGGGACAGGATGTG
ACACTGCAGGAGCTGGGACCAGAGGGCGACGATGTGGCCTTTCTGGTGTCCGAGGCCATCGG
CATCAGGATCAAGAATCCAGAGGCAATCGTGGTGCTGGAG
Linker
(SEQ ID NO: 29)
GGCGGCTCTGGCGGAAGTGGCGGAAGTGGGGGAAGTGGAGGCGGCGGAAGCGGGGGAGGCAG
CGGGGGAGGG
Protein sequence IgE leader - RBE- linker - HIV antigen
(SEQ ID NO: 30)
MDWTWILFLVAAATRVHSLSIAPTLINRDKPYTKEELMEILRLAIIAELDAINLYEQMARYS
EDENVRKILLDVAREEKAHVGEFMALLLNLDPEQVTELKGGFEEVKELTGIEAHINDNKKEE
SNVEYFEKLRSALLDGVNKGRSLLKHLPVTRIEGQSFRVDIIKFEDGVRVVKQEYKPIPLLK
KKFYVGIRELNDGTYDVSIATKAGELLVKDEESLVIREILSTEGIKKMKLSSWDNPEEALND
LMNALQEASNASAGPFGLIINPKRYAKLLKIYEKSGKMLVEVLKEIFRGGIIVTLNIDENKV
IIFANTPAVLDVVVGQDVTLQELGPEGDDVAFLVSEAIGIRIKNPEAIVVLEGGSGGSGGSG
GSGGGGSGGGSGGGDTITLPCRPAPPPHCSSNITGLILTRQGGYSNDNTVIFRPSGGDWRDI
ARCQIAGTVVSTQLFLNGSLAEEEVVIRSEDWRDNAKSICVQLNTSVEINCTGAGHCNISRA
KWNNTLKQIASKLREQYGNKTIIFKPSSGGDPEFVNHSFNCGGEFFYCDSTQLFNSTWFNST
RBE scaffold amino acid sequence
(SEQ ID NO: 31)
LSIAPTLINRDKPYTKEELMEILRLAIIAELDAINLYEQMARYSEDENVRKILLDVAREEKA
HVGEFMALLLNLDPEQVTELKGGFEEVKELTGIEAHINDNKKEESNVEYFEKLRSALLDGVN
KGRSLLKHLPVTRIEGQSFRVDIIKFEDGVRVVKQEYKPIPLLKKKFYVGIRELNDGTYDVS
IATKAGELLVKDEESLVIREILSTEGIKKMKLSSWDNPEEALNDLMNALQEASNASAGPFGL
IINPKRYAKLLKIYEKSGKMLVEVLKEIFRGGIIVTLNIDENKVIIFANTPAVLDVVVGQDV
TLQELGPEGDDVAFLVSEAIGIRIKNPEAIVVLE
Linker
(SEQ ID NO: 32)
GGSGGSGGSGGSGGGGSGGGSGGG
Nipah virus - Construct 1. NivFtop_stab2_gMax_Nt_60 mer
Entire Expressible Nucleic Acid Sequence for Nipah Virus antigen with 60 mer
Self-Assembly
(SEQ ID NO: 33)
ATGGACTGGACCTGGATTCTGTTCCTGGTGGCCGCCGCCACAAGGGTGCACAGCATGCAGAT
CTACGAAGGAAAACTGACCGCTGAGGGACTGAGGTTCGGAATTGTCGCAAGCCGCGCGAATC
ACGCACTGGTGGATAGGCTGGTGGAAGGCGCTATCGACGCAATTGTCCGGCACGGCGGGAGA
GAGGAAGACATCACACTGGTGAGAGTCTGCGGCAGCTGGGAGATTCCCGTGGCAGCTGGAGA
ACTGGCTCGAAAGGAGGACATCGATGCCGTGATCGCTATTGGGGTCCTGTGCCGAGGAGCAA
CTCCCAGCTTCGACTACATCGCCTCAGAAGTGAGCAAGGGGCTGGCTGATCTGTCCCTGGAG
CTGAGGAAACCTATCACTTTTGGCGTGATTACTGCCGACACCCTGGAACAGGCAATCGAGGC
GGCCGGCACCTGCCATGGAAACAAAGGCTGGGAAGCAGCCCTGTGCGCTATTGAGATGGCAA
ATCTGTTCAAATCTCTGCGAGGAGGCTCCGGAGGATCTGGAGGGAGTGGAGGCTCAGGAGGA
GGCGGGGTCACTTGTGCCGGACGAGCCATCGGAAATGCTACCGCCGCCCAGATTACTGCCGG
AGTCGCCCTGTATGAAGCCATGAAgAATGCCGACAACATCAATAAGCTGAAGAGCTCCATCG
AGAGCACCAACGAGGCCGTGGTGAAGCTGCAGGAGACAGCCGAgAAgACAGTGTACGTGCTG
ACAGCCCTGCAGGACTATATCAACACCAATCTGGTGCCCACAATCGATAAGATCAGCTGCAA
GCAGACCGAGGCATCCCTGGACGCCGCCCTGTCCAAGTACCTGTCTGATCTGCTGTACGTGT
TCGGCCCCAACCTGAGCGACCCCGTGAGCAATTCTATGCCTATCCAGGCCATCTCTCAGGCC
TTCGGCGGCAACTACAGCACCCTGCTGAGGACACTGGGCTATGCCCCAGAGGACTTTGACGA
TCTGCTGGAGAGCGATTCCATCACAGGCCAGATCATCTACGTGGACCTGTCTAGCTACTATA
TCATCGTGAGAGTGTATTTTCCAAATGGCTCCGGCCCCCTGACCAAGGATATCGTGATCAAG
ATGATCCCCAACGTGTCTAATATGAGCCAGTGTACAGGCTCTGTGATGGAGAACTACAAGAC
CAGGCTGAATGGCATCCTGACACCTATCAAGGGCGCCCTGGAGATCTATAAGAATAACTGTC
ACGATGGATGATAA
NivFtop_stab2_gMax
(SEQ ID NO: 34)
GGGGTCACTTGTGCCGGACGAGCCATCGGAAATGCTACCGCCGCCCAGATTACTG
CCGGAGTCGCCCTGTATGAAGCCATGAAgAATGCCGACAACATCAATAAGCTGAAGAGCTCC
ATCGAGAGCACCAACGAGGCCGTGGTGAAGCTGCAGGAGACAGCCGAgAAgACAGTGTACGT
GCTGACAGCCCTGCAGGACTATATCAACACCAATCTGGTGCCCACAATCGATAAGATCAGCT
GCAAGCAGACCGAGGCATCCCTGGACGCCGCCCTGTCCAAGTACCTGTCTGATCTGCTGTAC
GTGTTCGGCCCCAACCTGAGCGACCCCGTGAGCAATTCTATGCCTATCCAGGCCATCTCTCA
GGCCTTCGGCGGCAACTACAGCACCCTGCTGAGGACACTGGGCTATGCCCCAGAGGACTTTG
ACGATCTGCTGGAGAGCGATTCCATCACAGGCCAGATCATCTACGTGGACCTGTCTAGCTAC
TATATCATCGTGAGAGTGTATTTTCCAAATGGCTCCGGCCCCCTGACCAAGGATATCGTGAT
CAAGATGATCCCCAACGTGTCTAATATGAGCCAGTGTACAGGCTCTGTGATGGAGAACTACA
AGACCAGGCTGAATGGCATCCTGACACCTATCAAGGGCGCCCTGGAGATCTATAAGAATAAC
TGTCACGATGGATGATAA
Entire Expressed IgE- Self Assembly-Linker - Viral
Antigen sequence
(SEQ ID NO: 35)
MDWTWILFLVAAATRVHSMQIYEGKLTAEGLRFGIVASRANHALVDRLVEGAIDAIVRHGGR
EEDITLVRVCGSWEIPVAAGELARKEDIDAVIAIGVLCRGATPSFDYIASEVSKGLADLSLE
LRKPITFGVITADTLEQAIEAAGTCHGNKGWEAALCAIEMANLFKSLRGGSGGSGGSGGSGG
GGVTCAGRAIGNATAAQITAGVALYEAMKNADNINKLKSSIESTNEAVVKLQETAEKTVYVL
TALQDYINTNLVPTIDKISCKQTEASLDAALSKYLSDLLYVFGPNLSDPVSNSMPIQAISQA
FGGNYSTLLRTLGYAPEDFDDLLESDSITGQIIYVDLSSYYIIVRVYFPNGSGPLTKDIVIK
MIPNVSNMSQCTGSVMENYKTRLNGILTPIKGALEIYKNNCHDG
NivFtop_stab2_gMax expressed amino acid sequence
(SEQ ID NO: 36)
GVTCAGRAIGNATAAQITAGVALYEAMKNADNINKLKSSIESTNEAVVKLQETAEKTVYVLT
ALQDYINTNLVPTIDKISCKQTEASLDAALSKYLSDLLYVFGPNLSDPVSNSMPIQAISQAF
GGNYSTLLRTLGYAPEDFDDLLESDSITGQIIYVDLSSYYIIVRVYFPNGSGPLTKDIVIKM
IPNVSNMSQCTGSVMENYKTRLNGILTPIKGALEIYKNNCHDG
Construct 2.
NivFtop_stab2_gMax_Ct_60 mer
Entire Expressible Nucleic Acid Sequence for Nipah Virus
Antigen with 60 mer Self-Assembly
(SEQ ID NO: 37)
ATGGACTGGACCTGGATTCTGTTCCTGGTGGCCGCCGCCACAAGGGTGCACAGCGGGGTCAC
TTGTGCCGGACGAGCCATCGGAAATGCTACCGCCGCCCAGATTACTGCCGGAGTCGCCCTGT
ATGAAGCCATGAAgAATGCCGACAACATCAATAAGCTGAAGAGCTCCATCGAGAGCACCAAC
GAGGCCGTGGTGAAGCTGCAGGAGACAGCCGAgAAgACAGTGTACGTGCTGACAGCCCTGCA
GGACTATATCAACACCAATCTGGTGCCCACAATCGATAAGATCAGCTGCAAGCAGACCGAGG
CATCCCTGGACGCCGCCCTGTCCAAGTACCTGTCTGATCTGCTGTACGTGTTCGGCCCCAAC
CTGAGCGACCCCGTGAGCAATTCTATGCCTATCCAGGCCATCTCTCAGGCCTTCGGCGGCAA
CTACAGCACCCTGCTGAGGACACTGGGCTATGCCCCAGAGGACTTTGACGATCTGCTGGAGA
GCGATTCCATCACAGGCCAGATCATCTACGTGGACCTGTCTAGCTACTATATCATCGTGAGA
GTGTATTTTCCAAATGGCTCCGGCCCCCTGACCAAGGATATCGTGATCAAGATGATCCCCAA
CGTGTCTAATATGAGCCAGTGTACAGGCTCTGTGATGGAGAACTACAAGACCAGGCTGAATG
GCATCCTGACACCTATCAAGGGCGCCCTGGAGATCTATAAGAATAACTGTCACGATGGAGGA
GGCTCCGGAGGATCTGGAGGGAGTGGAGGCTCAGGAGGAGGCATGCAGATCTACGAAGGAAA
ACTGACCGCTGAGGGACTGAGGTTCGGAATTGTCGCAAGCCGCGCGAATCACGCACTGGTGG
ATAGGCTGGTGGAAGGCGCTATCGACGCAATTGTCCGGCACGGCGGGAGAGAGGAAGACATC
ACACTGGTGAGAGTCTGCGGCAGCTGGGAGATTCCCGTGGCAGCTGGAGAACTGGCTCGAAA
GGAGGACATCGATGCCGTGATCGCTATTGGGGTCCTGTGCCGAGGAGCAACTCCCAGCTTCG
ACTACATCGCCTCAGAAGTGAGCAAGGGGCTGGCTGATCTGTCCCTGGAGCTGAGGAAACCT
ATCACTTTTGGCGTGATTACTGCCGACACCCTGGAACAGGCAATCGAGGCGGCCGGCACCTG
CCATGGAAACAAAGGCTGGGAAGCAGCCCTGTGCGCTATTGAGATGGCAAATCTGTTCAAAT
CTCTGCGATGATAA
Entire Expressible Amino Acid Sequence for Nipah Virus
Antigen with 60 mer Self-Assembly
(SEQ ID NO: 38)
MDWTWILFLVAAATRVHSGVTCAGRAIGNATAAQITAGVALYEAMKNADNINKLKSSIESTN
EAVVKLQETAEKTVYVLTALQDYINTNLVPTIDKISCKQTEASLDAALSKYLSDLLYVFGPN
LSDPVSNSMPIQAISQAFGGNYSTLLRTLGYAPEDFDDLLESDSITGQIIYVDLSSYYIIVR
VYFPNGSGPLIKDIVIKMIPNVSNMSQCTGSVMENYKTRLNGILTPIKGALEIYKNNCHDGG
GSGGSGGSGGSGGGMQIYEGKLTAEGLRFGIVASRANHALVDRLVEGAIDAIVRHGGREEDI
TLVRVCGSWEIPVAAGELARKEDIDAVIAIGVLCRGATPSFDYIASEVSKGLADLSLELRKP
ITFGVITADTLEQAIEAAGTCHGNKGWEAALCAIEMANLFKSLR
Influenza
Construct 1 - NC99_60 mer pVax
Entire expressible nucleic acid sequence
(SEQ ID NO: 57)
GGATCCGCCACCATGGACTGGACCTGGATTCTGTTCCTGGTGGCCGCCGCCACAAGGGTGCA
CAGCATGCAGATCTACGAAGGAAAACTGACCGCTGAGGGACTGAGGTTCGGAATTGTCGCAA
GCCGCGCGAATCACGCACTGGTGGATAGGCTGGTGGAAGGCGCTATCGACGCAATTGTCCGG
CACGGCGGGAGAGAGGAAGACATCACACTGGTGAGAGTCTGCGGCAGCTGGGAGATTCCCGT
GGCAGCTGGAGAACTGGCTCGAAAGGAGGACATCGATGCCGTGATCGCTATTGGGGTCCTGT
GCCGAGGAGCAACTCCCAGCTTCGACTACATCGCCTCAGAAGTGAGCAAGGGGCTGGCTGAT
CTGTCCCTGGAGCTGAGGAAACCTATCACTTTTGGCGTGATTACTGCCGACACCCTGGAACA
GGCAATCGAGGCGGCCGGCACCTGCCATGGAAACAAAGGCTGGGAAGCAGCCCTGTGCGCTA
TTGAGATGGCAAATCTGTTCAAATCTCTGCGAGGAGGCTCCGGAGGATCTGGAGGGAGTGGA
GGCTCAGGAGGAGGCGCCCCTCTGCAGCTGGGAAACTGCTCCGTGGCAGGATGGATTCTGGG
CAATCCAGAGTGTGAGCTGCTGATCTCTAAGGAGTCCTGGTCTTACATCGTGGAGACCCCAA
ACCCCGAGAATGGCACATGCTTTCCCGGCTACTTCGCCGACTATGAGGAGCTGAGGGAGCAG
CTGAGCTCCGTGTCTAGCTTCGAGAGATTTGAGATCTTCCCTAAGGAGTCCTCTTGGCCAAA
CCACACCGTGACAGGCGTGAGCGCCTCCTGTTCTCACAACGGCAAGAGCTCCTTTTATAGGA
ATCTGCTGTGGCTGACCGGCAAGAACGGCCTGTACCCTAATCTGAGCAAGTCCTATGTGAAC
AATAAGGAGAAGGAGGTGCTGGTGCTGTGGGGCGTGCACCACCCTCCCAACATCGGCAATCA
GAGGGCCCTGTACCACACCGAGAACGCCTACGTGAGCGTGGTGTCTAGCCACTACAGCAGGA
GATTCACACCCGAGATCGCCAAGAGGCCTAAGGTGCGCGACCAGGAGGGACGGATCAATTAC
TATTGGACCCTGCTGGAGCCAGGCGATACAATCATCTTTGAGGCCAACGGCAATCTGATCGC
CCCCTGGTATGCCTTCGCCCTGTCCCGCGGCTGATAACTCGAG
Entire expressible amino acid sequence
(SEQ ID NO: 58)
MDWTWILFLVAAATRVHSMQIYEGKLTAEGLRFGIVASRANHALVDRLVEGAIDAIVRHGGR
EEDITLVRVCGSWEIPVAAGELARKEDIDAVIAIGVLCRGATPSFDYIASEVSKGLADLSLE
LRKPITFGVITADTLEQATEAAGTCHGNKGWEAALCAIEMANLFKSLRGGSGGSGGSGGSGG
GAPLQLGNCSVAGWILGNPECELLISKESWSYIVETPNPENGTCFPGYFADYEELREQLSSV
SSFERFEIFPKESSWPNHTVTGVSASCSHNGKSSFYRNLLWLTGKNGLYPNLSKSYVNNKEK
EVLVLWGVHHPPNIGNQRALYHTENAYVSVVSSHYSRRFTPEIAKRPKVRDQEGRINYYWIL
LEPGDTIIFEANGNLIAPWYAFALSRG
Construct 2 - NC99_g6_60 mer_pVax
Entire expressible nucleic acid sequence
(SEQ ID NO: 59)
GGATCCGCCACCATGGACTGGACCTGGATTCTGTTCCTGGTGGCCGCCGCCACAAGGGTGCA
CAGCATGCAGATCTACGAAGGAAAACTGACCGCTGAGGGACTGAGGTTCGGAATTGTCGCAA
GCCGCGCGAATCACGCACTGGTGGATAGGCTGGTGGAAGGCGCTATCGACGCAATTGTCCGG
CACGGCGGGAGAGAGGAAGACATCACACTGGTGAGAGTCTGCGGCAGCTGGGAGATTCCCGT
GGCAGCTGGAGAACTGGCTCGAAAGGAGGACATCGATGCCGTGATCGCTATTGGGGTCCTGT
GCCGAGGAGCAACTCCCAGCTTCGACTACATCGCCTCAGAAGTGAGCAAGGGGCTGGCTGAT
CTGTCCCTGGAGCTGAGGAAACCTATCACTTTTGGCGTGATTACTGCCGACACCCTGGAACA
GGCAATCGAGGCGGCCGGCACCTGCCATGGAAACAAAGGCTGGGAAGCAGCCCTGTGCGCTA
TTGAGATGGCAAATCTGTTCAAATCTCTGCGAGGAGGCTCCGGAGGATCTGGAGGGAGTGGA
GGCTCAGGAGGAGGCGCCCCTCTGCAGCTGGGAAACTGCAGCGTGGCAGGATGGATTCTGGG
CAATCCAGAGTGTGAGCTGCTGATCTCCAAGGAGTCCTGGTCTTACATCGTGGAGACCCCAA
ACCCCGAGAATGGCACATGCTTTCCCGGCAACTTCTCTGACTATGAGGAGCTGAGGGAGCAG
CTGAGCTCCGTGTCTAGCTTCGAGAGATTTGAGATCTTCCCTAAGGAGTCCTCTTGGCCAAA
TCACACCGTGACAGGCGTGAGCGCCTCCTGTTCTCACAACGGCAAGAGCTCCTTTTACAGGA
ATCTGCTGTGGCTGACCGGCAAGAACGGCCTGTACCCTAATCTGAGCAAGTCCTATAACAAT
ACAAAGGAGAAGGAGGTGCTGGTGCTGTGGGGCGTGCACCACCCTCCCAACATCGGCAATCA
GAGGGCCCTGTACCACACCGAGAACGCCTACGTGAGCGTGGTGTCTAGCCACTACTCTAGGA
GATTCACACCCAACATCAGCAAGAGGCCTAAGGTGCGCGACCAGGAGGGACGGATCAATTAC
TATTGGACCCTGCTGGAGCCAGGCGATACAATCATCTTTGAGGCCAACGGCAATCTGATCGC
CCCCTGGTATGCCTTCGCCCTGTCTCGCGGCAACGGCAGCTGATAACTCGAG
Entire expressible amino acid sequence
(SEQ ID NO: 60)
MDWTWILFLVAAATRVHSMQIYEGKLTAEGLRFGIVASRANHALVDRLVEGAIDAIVRHGGR
EEDITLVRVCGSWEIPVAAGELARKEDIDAVIAIGVLCRGATPSFDYIASEVSKGLADLSLE
LRKPITFGVITADTLEQAIEAAGTCHGNKGWEAALCAIEMANLFKSLRGGSGGSGGSGGSGG
GAPLQLGNCSVAGWILGNPECELLISKESWSYIVETPNPENGTCFPGNFSDYEELREQLSSV
SSFERFEIFPKESSWPNHTVTGVSASCSHNGKSSFYRNLLWLTGKNGLYPNLSKSYNNTKEK
EVLVLWGVHHPPNIGNQRALYHTENAYVSVVSSHYSRRFTPNISKRPKVRDQEGRINYYWTL
LEPGDTIIFEANGNLIAPWYAFALSRGNGS
Construct 3 - CA09(175L)_Ferritin_pVax
Entire expressible nucleic acid sequence
(SEQ ID NO: 61)
ATGGACTGGACTTGGATTCTGTTTCTGGTCGCCGCTGCCACTCGCGTGCATTCTGCCCCACT
GCACCTGGGCAAGTGCAACATCGCCGGCTGGATTCTGGGCAATCCCGAGTGTGAGAGCCTGT
CCACCGCCAGCTCCTGGAGCTACATCGTGGAGACCCCTTCTAGCGACAACGGCACATGCTTT
CCAGGCGACTTCATCGATTATGAGGAGCTGAGGGAGCAGCTGTCCTCTGTGAGCTCCTTCGA
GAGATTTGAGATCTTCCCCAAGACCTCTAGCTGGCCTAACCACGATTCCAATAAGGGAGTGA
CAGCAGCATGTCCTCACGCAGGCGCCAAGAGCTTTTACAAGAACCTGATCTGGCTGGTGAAG
AAGGGCAATTCCTACCCAAAGCTGTCTAAGAGCTATATCAACGACAAGGGCAAGGAGGTGCT
GGTGCTGTGGGGCATCCACCACCCATCCACCTCTGCCGACCAGCAGTCTCTGTACCAGAATG
CCGATACATACGTGTTCGTGGGCTCCTCTCGGTACTCCAAGAAGTTCAAGCCAGAGATCGCC
ATCAGGCCCAAGGTGAGAGACCAGGAGGGCCGCATGAATTACTATTGGACACTGGTGGAGCC
CGGCGATAAGATCACCTTTGAGGCCACAGGCAACCTGGTGGTGCCTCGGTATGCCTTCGCCA
TGGAGCGCAATGCAAGCGGGGAAAGCCAGGTGCGACAGCAGTTCTCCAAAGACATCGAAAAG
CTGCTGAATGAACAGGTCAACAAGGAAATGCAGAGCAGCAACCTGTACATGTCCATGAGCTC
CTGGTGCTATACCCACTCTCTGGACGGAGCAGGCCTGTTCCTGTTTGATCACGCCGCCGAGG
AGTACGAGCACGCCAAGAAGCTGATCATCTTCCTGAATGAGAACAATGTGCCCGTGCAGCTG
ACCTCTATCAGCGCCCCTGAGCACAAGTTCGAGGGCCTGACACAGATCTTTCAGAAGGCCTA
CGAGCACGAGCAGCACATCTCCGAGTCTATCAACAATATCGTGGACCACGCCATCAAGTCCA
AGGATCACGCCACATTCAACTTTCTGCAGTGGTACGTGGCCGAGCAGCACGAGGAGGAGGTG
CTGTTTAAGGACATCCTGGATAAGATCGAGCTGATCGGCAACGAGAATCACGGGCTGTATCT
GGCCGACCAGTATGTGAAGGGCATCGCTAAAAGCAGGAAATCAGGAAGC
Entire expressible amino acid sequence
(SEQ ID NO: 62)
MDWTWILFLVAAATRVHSAPLHLGKCNIAGWILGNPECESLSTASSWSYIVETPSSDNGTCF
PGDFIDYEELREQLSSVSSFERFEIFPKTSSWPNHDSNKGVTAACPHAGAKSFYKNLIWLVK
KGNSYPKLSKSYINDKGKEVLVLWGIHHPSTSADQQSLYQNADTYVFVGSSRYSKKFKPEIA
IRPKVRDQEGRMNYYWTLVEPGDKITFEATGNLVVPRYAFAMERNASGESQVRQQFSKDIEK
LLNEQVNKEMQSSNLYMSMSSWCYTHSLDGAGLFLFDHAAEEYEHAKKLIIFLNENNVPVQL
TSISAPEHKFEGLTQIFQKAYEHEQHISESINNIVDHAIKSKDHATFNFLQWYVAEQHEEEV
LFKDILDKIELIGNENHGLYLADQYVKGIAKSRKSGS
Construct 4 - H1_CA04/09_FL_HA_3BVE_pVAX
Entire expressible nucleic acid sequence
(SEQ ID NO: 63)
ATGGACTGGACTTGGATTCTGTTCCTGGTCGCCGCCGCAACCCGCGTGCATTCTATGAAGGC
TATTCTGGTCGTGCTGCTGTATACTTTCGCCACCGCCAACGCCGACACACTGTGCATCGGCT
ACCACGCCAACAATTCTACCGACACAGTGGATACCGTGCTGGAGAAGAATGTGACCGTGACA
CACAGCGTGAACCTGCTGGAGGATAAGCACAATGGCAAGCTGTGCAAGCTGAGGGGAGTGGC
ACCACTGCACCTGGGCAAGTGCAACATCGCCGGCTGGATTCTGGGCAATCCCGAGTGTGAGT
CCCTGTCTACAGCCAGCTCCTGGTCCTACATCGTGGAGACACCCTCTAGCGACAACGGCACA
TGCTACCCTGGCGACTTTATCGATTATGAGGAGCTGCGGGAGCAGCTGAGCAGCGTGAGCAG
CTTCGAGAGGTTCGAGATCTTCCCCAAGACCTCTAGCTGGCCTAACCACGATAGCAATAAGG
GAGTGACAGCAGCATGTCCACACGCAGGCGCCAAGAGCTTCTATAAGAACCTGATCTGGCTG
GTGAAGAAGGGCAATTCCTACCCTAAGCTGAGCAAGTCCTATATCAACGACAAGGGCAAGGA
GGTGCTGGTGCTGTGGGGCATCCACCACCCATCTACCAGCGCCGACCAGCAGTCCCTGTACC
AGAATGCCGATACATACGTGTTCGTGGGCTCCTCTCGGTACTCTAAGAAGTTCAAGCCAGAG
ATCGCCATCAGGCCAAAGGTGAGGGACCAGGAGGGACGCATGAACTACTATTGGACCCTGGT
GGAGCCCGGCGATAAGATCACCTTTGAGGCCACAGGCAACCTGGTGGTGCCTAGATATGCCT
TCGCCATGGAGAGAAATGCCGGCTCCGGCATCATCATCTCTGACACCCCTGTGCACGATTGC
AACACCACATGTCAGACCCCAAAGGGCGCCATCAACACATCCCTGCCTTTTCAGAATATCCA
CCCAATCACAATCGGCAAGTGCCCTAAGTACGTGAAGAGCACCAAGCTGAGGCTGGCAACAG
GCCTGCGCAATATCCCATCTATCCAGAGCAGGGGCCTGTTTGGAGCAATCGCAGGCTTCATC
GAGGGAGGATGGACCGGAATGGTGGACGGCTGGTACGGCTATCACCACCAGAACGAGCAGGG
CAGCGGATATGCAGCAGACCTGAAGTCCACCCAGAATGCCATCGATGAGATCACAAACAAGG
TCAATTCCGTGATCGAGAAGATGAACACCCAGTTTACAGCCGTGGGCAAGGAGTTCAATCAC
CTGGAGAAGAGAATCGAGAACCTGAATAAGAAGGTGGACGATGGCTTCCTGGACATCTGGAC
CTACAACGCCGAGCTGCTGGTGCTGCTGGAGAATGAGAGGACACTGGACTACCACGATTCCA
ACGTGAAGAATCTGTATGAGAAGGTGAGATCTCAGCTGAAGAACAATGCCAAGGAGATCGGC
AACGGCTGCTTCGAGTTTTACCACAAGTGCGACAACACCTGTATGGAGAGCGTGAAGAATGG
CACATACGATTATCCTAAGTATTCCGAGGAGGCCAAGCTGAACCGCGAGGAGATCGACTCTG
GCGGCGATATCATCAAGCTGCTGAACGAGCAAGTGAATAAGGAGATGCAGAGCTCCAATCTG
TACATGTCTATGTCTAGCTGGTGTTATACCCACAGCCTGGACGGAGCAGGCCTGTTCCTGTT
TGATCACGCCGCCGAGGAGTACGAGCACGCCAAGAAGCTGATCATCTTTCTGAACGAGAACA
ATGTGCCAGTGCAGCTGACCTCCATCTCTGCCCCCGAGCACAAGTTTGAGGGCCTGACACAG
ATCTTCCAGAAGGCCTACGAGCACGAGCAGCACATCAGCGAGTCCATCAACAATATCGTGGA
CCACGCCATCAAGAGCAAGGATCACGCCACCTTCAACTTTCTGCAGTGGTACGTGGCCGAGC
AGCACGAGGAGGAGGTGCTGTTCAAGGACATCCTGGATAAGATCGAGCTGATCGGCAACGAG
AATCACGGGCTGTACCTGGCAGACCAGTATGTCAAGGGCATCGCAAAGTCACGGAAGAGCGG
GAGC
Entire expressible amino acid sequence
(SEQ ID NO: 64)
MDWTWILFLVAAATRVHSMKAILVVLLYTFATANADTLCIGYHANNSTDTVDTVLEKNVTVT
HSVNLLEDKHNGKLCKLRGVAPLHLGKCNIAGWILGNPECESLSTASSWSYIVETPSSDNGT
CYPGDFIDYEELREQLSSVSSFERFEIFPKTSSWPNHDSNKGVTAACPHAGAKSFYKNLIWL
VKKGNSYPKLSKSYINDKGKEVLVLWGIHHPSTSADQQSLYQNADTYVFVGSSRYSKKFKPE
IAIRPKVRDQEGRMNYYWTLVEPGDKITFEATGNLVVPRYAFAMERNAGSGIIISDTPVHDC
NTTCQTPKGAINTSLPFQNIHPITIGKCPKYVKSTKLRLATGLRNIPSIQSRGLFGAIAGFI
EGGWTGMVDGWYGYHHQNEQGSGYAADLKSTQNAIDEITNKVNSVIEKMNTQFTAVGKEFNH
LEKRIENLNKKVDDGFLDIWTYNAELLVLLENERTLDYHDSNVKNLYEKVRSQLKNNAKEIG
NGCFEFYHKCDNTCMESVKNGTYDYPKYSEEAKLNREEIDSGGDIIKLLNEQVNKEMQSSNL
YMSMSSWCYTHSLDGAGLFLFDHAAEEYEHAKKLIIFLNENNVPVQLTSISAPEHKFEGLTQ
IFQKAYEHEQHISESINNIVDHAIKSKDHATFNFLQWYVAEQHEEEVLFKDILDKIELIGNE
NHGLYLADQYVKGIAKSRKSGS

### D. Pharmaceutical Compositions

Disclosed are pharmaceutical compositions comprising any one or more of the disclosed compositions and a pharmaceutically acceptable carrier.

In some embodiments, any of the disclosed compositions is from about 1 to about 30 micrograms. For example, any of the disclosed compositions can be from about 1 to about 5 micrograms. In some preferred embodiments, the pharmaceutical compositions contain from about 5 nanograms to about 800 micrograms of DNA. In some preferred embodiments, the pharmaceutical compositions contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of the consensus antigen or plasmid thereof. The pharmaceutical compositions can comprise from about 5 nanograms to about 10 mg of the vaccine DNA. In some embodiments, pharmaceutical compositions according to the present disclosure comprise from about 25 nanogram to about 5 mg of vaccine DNA. In some embodiments, the pharmaceutical compositions contain from about 50 nanograms to about 1 mg of DNA. In some embodiments, the pharmaceutical compositions contain about from about 0.1 to about 500 micrograms of DNA. In some embodiments, the pharmaceutical compositions contain from about 1 to about 350 micrograms of DNA. In some embodiments, the pharmaceutical compositions contain from about 5 to about 250 micrograms of DNA. In some embodiments, the pharmaceutical compositions contain from about 10 to about 200 micrograms of DNA. In some embodiments, the pharmaceutical compositions contain from about 15 to about 150 micrograms of DNA. In some embodiments, the pharmaceutical compositions contain about 20 to about 100 micrograms of DNA. In some embodiments, the pharmaceutical compositions contain about 25 to about 75 micrograms of DNA. In some embodiments, the pharmaceutical compositions contain about 30 to about 50 micrograms of DNA. In some embodiments, the pharmaceutical compositions contain about 35 to about 40 micrograms of DNA. In some embodiments, the pharmaceutical compositions contain about 100 to about 200 microgram DNA. In some embodiments, the pharmaceutical compositions comprise about 10 microgram to about 100 micrograms of DNA. In some embodiments, the pharmaceutical compositions comprise about 20 micrograms to about 80 micrograms of DNA. In some embodiments, the pharmaceutical compositions comprise about 25 micrograms to about 60 micrograms of DNA. In some embodiments, the pharmaceutical compositions comprise about 30 nanograms to about 50 micrograms of DNA. In some embodiments, the pharmaceutical compositions comprise about 35 nanograms to about 45 micrograms of DNA. In some preferred embodiments, the pharmaceutical compositions contain about 0.1 to about 500 micrograms of DNA. In some preferred embodiments, the pharmaceutical compositions contain about 1 to about 350 micrograms of DNA. In some preferred embodiments, the pharmaceutical compositions contain about 1 to about 250 micrograms of DNA. In some preferred embodiments, the pharmaceutical compositions contain about 2 to about 200 microgram DNA.

In some embodiments, pharmaceutical compositions according to the present disclosure comprise at least about 10, 15, 20, 25, 30, 35, 40, 45, 50, 55, 60, 65, 70, 75, 80, 85, 90, 95 or 100 nanograms of DNA of the vaccine. In some embodiments, the pharmaceutical compositions can comprise at least about 1, 5, 10, 15, 20, 25, 30, 35, 40, 45, 50, 55, 60, 65, 70, 75, 80, 85, 90, 95,100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995 or 1000 micrograms of DNA of the vaccine. In some embodiments, the pharmaceutical composition can comprise at least 1.5, 2, 2.5, 3, 3.5, 4, 4.5, 5, 5.5, 6, 6.5, 7, 7.5, 8, 8.5, 9, 9.5 or 10 mg or more of DNA of the vaccine.

In other embodiments, the pharmaceutical composition can comprise up to and including about 10, 15, 20, 25, 30, 35, 40, 45, 50, 55, 60, 65, 70, 75, 80, 85, 90, 95 or 100 nanograms of DNA of the vaccine. In some embodiments, the pharmaceutical composition can comprise up to and including about 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995, or 1000 micrograms of DNA of the vaccine. In some embodiments, the pharmaceutical composition can comprise up to and including about 1.5, 2, 2.5, 3, 3.5, 4, 4.5, 5, 5.5, 6, 6.5, 7, 7.5, 8, 8.5, 9, 9.5 or about 10 mg of DNA of the vaccine. The pharmaceutical composition can further comprise other agents for formulation purposes according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The vaccine can further comprise a pharmaceutically acceptable excipient. The pharmaceutically acceptable excipient can be functional molecules as vehicles, adjuvants, carriers, or diluents. The pharmaceutically acceptable excipient can be a transfection facilitating agent, which can include surface active agents, such as immune-stimulating complexes

(ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or other known transfection facilitating agents. In some embodiments, the vaccine is a composition comprising a plasmid DNA molecule, RNA molecule or DNA/RNA hybrid molecule encoding an expressible nucleic acid sequence, the expressible nucleic acid sequence comprising a first nucleic acid encoding a self-assembling nanoparticle comprising a viral antigen, optionally encoding a leader sequence disclosed herein.

The transfection facilitating agent is a polyanion, polycation, including poly-L-glutamate (LGS), or lipid. The transfection facilitating agent is poly-L-glutamate, and more preferably, the poly-L-glutamate is present in the vaccine at a concentration less than 6 mg/ml. The transfection facilitating agent can also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid can also be used administered in conjunction with the genetic construct. In some embodiments, the DNA vector vaccines can also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant. The adjuvant can be other genes that are expressed in alternative plasmid or are deneurological systemed as proteins in combination with the plasmid above in the vaccine. The adjuvant can be selected from the group consisting of: α-interferon(IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof. In an exemplary embodiment, the adjuvant is IL-12.

Other genes which can be useful adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof or a combination thereof.

In some embodiments adjuvant may be one or more proteins and/or nucleic acid molecules that encode proteins selected from the group consisting of: CCL-20, IL-12, IL-15, IL-28, CTACK, TECK, MEC or RANTES. Examples of IL-12 constructs and sequences are disclosed in PCT application no. PCT/US 1997/019502 and corresponding U.S. application Ser. No. 08/956,865, and U.S. Provisional Application Ser. No. 61/569,600 filed Dec. 12, 2011, which are each incorporated herein by reference in their entireties. Examples of IL-15 constructs and sequences are disclosed in PCT application no. PCT/US04/18962 and corresponding U.S. application Ser. No. 10/560,650, and in PCT application no. PCT/US07/00886 and corresponding U.S. application Ser. No. 12/160,766, and in PCT Application Serial No. PCT/US10/048827, which are each incorporated herein by reference in their entireties. Examples of IL-28 constructs and sequences are disclosed in PCT application no. PCT/US09/039648 and corresponding U.S. application Ser. No. 12/936,192, which are each incorporated herein by reference in their entireties. Examples of RANTES and other constructs and sequences are disclosed in PCT application no. PCT/US 1999/004332 and corresponding U.S. application Ser. No. 09/622,452, which are each incorporated herein by reference in their entities. Other examples of RANTES constructs and sequences are disclosed in PCT Application Serial No. PCT/US Serial No. 11/024098, which is incorporated herein by reference. Examples of RANTES and other constructs and sequences are disclosed in PCT Application Serial No. PCT/US 1999/004332 and corresponding U.S. application Ser. No. 09/622,452, which are each incorporated herein by reference. Other examples of RANTES constructs and sequences are disclosed in PCT application no. PCT/US11/024098, which is incorporated herein by reference in its entirety. Examples of chemokines CTACK, TECK and MEC constructs and sequences are disclosed in PCT Application Serial No. PCT/US2005/042231 and corresponding U.S. application Ser. No. 11/719,646, which are each incorporated herein by reference in their entireties. Examples of OX40 and other immunomodulators are disclosed in U.S. application Ser. No. 10/560,653, which is incorporated herein by reference in its entirety. Examples of DR5 and other immunomodulators are disclosed in U.S. application Ser. No. 09/622,452, which is incorporated herein by reference in its entirety.

The pharmaceutical composition may be formulated according to the mode of administration to be used. An injectable vaccine pharmaceutical composition may be sterile, pyrogen free and particulate free. An isotonic formulation or solution may be used. Additives for isotonicity may include sodium chloride, dextrose, mannitol, sorbitol, and lactose. The vaccine may comprise a vasoconstriction agent. The isotonic solutions may include phosphate buffered saline. Vaccine may further comprise stabilizers including gelatin and albumin. The stabilizing may allow the formulation to be stable at room or ambient temperature for extended periods of time such as LGS or polycations or polyanions to the vaccine formulation.

The vaccine can be a DNA vaccine. DNA vaccines are disclosed in U.S. Pat. Nos. 5,593,972, 5,739,118, 5,817,637, 5,830,876, 5,962,428, 5,981,505, 5,580,859, 5,703,055, and 5,676,594, which are incorporated herein fully by reference. The DNA vaccine can further comprise elements or reagents that inhibit it from integrating into the chromosome. Examples of attenuated live vaccines, those using recombinant vectors to foreign antigens, subunit vaccines and glycoprotein vaccines are described in U.S. Pat. Nos. 4,510,245; 4,797,368; 4,722,848; 4,790,987; 4,920,209; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,364; 5,462,734; 5,470,734; 5,474,935; 5,482,713; 5,591,439; 5,643,579; 5,650,309; 5,698,202; 5,955,088; 6,034,298; 6,042,836; 6,156,319 and 6,589,529, which are each incorporated herein by reference in their entireties.

The genetic construct can also be part of a genome of a recombinant viral vector, including recombinant adenovirus, recombinant adenovirus associated virus and recombinant vaccinia. The genetic construct can be part of the genetic material in attenuated live microorganisms or recombinant microbial vectors which live in cells.

The disclosure relates to a genetic construct or composition comprising a first, second, third or more nucleic acid molecule, each of the first, second or third nucleic acid molecules comprising an expressible nucleic acid sequence that encodes a self-assembling polypeptide and/or a viral antigen and/or a leader sequence optionally fused by a linker. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:1. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:2. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:3. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:4. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:5. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:6. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:7. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:8. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:9. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:10. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:11. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:12. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:13. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:14. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:15. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:16. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:17. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:18. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:19. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:20. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:21. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:22. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:23. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:24. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:25. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:26. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:27. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:28. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:29. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:30. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:31. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:32. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:33. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:34. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:35. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:36. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:37. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:38. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:39. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:40. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:41. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:42. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:43. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:44. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:45. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:46. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:47. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:48. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:49. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:50. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:51. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:52. In some embodiments, the disclosure relates to a composition, such as a pharmaceutical composition comprising an expressible nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to any one or combination of SEQ ID NO:1 through SEQ ID NO:53. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:54. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:55. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO56. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:57. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:58. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:59. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:60. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:61. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:62. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:63. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:64. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:65. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:66. In some embodiments the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO:67.

### E. Methods

Disclosed are methods of vaccinating a subject comprising administering a therapeutically effective amount of any of the disclosed nucleic acid molecules, compositions, cells or pharmaceutical compositions to the subject. In some embodiments, the vaccination is against viral infection. In some embodiments, the viral infection is an infection of retroviridae. In some embodiments, the viral infection is an infection of a Flavivirus. In some embodiments, the viral infection is an infection of Nipah Virus. In some embodiments, the viral infection is an infection of West Nile virus. In some embodiments, the viral infection is an infection of human papillomavirus. In some embodiments, the viral infection is an infection of respiratory syncytial virus. In some embodiments, the viral infection is an infection of filovirus. In some embodiments, the viral infection is an infection of zaire ebolavirus. In some embodiments, the viral infection is an infection of sudan ebolavirus. In some embodiments, the viral infection is an infection of marburgvirus. In some embodiments, the viral infection is an infection of influenza virus.

Disclosed are methods of inducing an immune response in a subject comprising administering to the subject any of the disclosed pharmaceutical compositions. In some embodiments, the methods are for inducing an immune response to a viral antigen in the subject. In some embodiments, the immune response induced by the disclosed methods is against a viral antigen from a retroviridae. In some embodiments, the immune response induced by the disclosed methods is against a viral antigen from a flavivirus. In some embodiments, the immune response induced by the disclosed methods is against a viral antigen from a Nipah Virus. In some embodiments, the immune response induced by the disclosed methods is against a viral antigen from a West Nile virus. In some embodiments, the immune response induced by the disclosed methods is against a viral antigen from a human papillomavirus. In some embodiments, the immune response induced by the disclosed methods is against a viral antigen from a respiratory syncytial virus. In some embodiments, the immune response induced by the disclosed methods is against a viral antigen from a filovirus. In some embodiments, the immune response induced by the disclosed methods is against a viral antigen from a zaire ebolavirus. In some embodiments, the immune response induced by the disclosed methods is against a viral antigen from a sudan ebolavirus. In some embodiments, the immune response induced by the disclosed methods is against a viral antigen from a marburgvirus. In some embodiments, the immune response induced by the disclosed methods is against a viral antigen from an influenza virus.

Disclosed are methods of neutralizing one or a plurality of viruses in a subject comprising administering to the subject any of the disclosed pharmaceutical compositions. In some embodiments, the virus being neutralized by the disclosed method is retroviridae. In some embodiments, the virus being neutralized by the disclosed method is flavivirus. In some embodiments, the virus being neutralized by the disclosed method is Nipah Virus. In some embodiments, the virus being neutralized by the disclosed method is West Nile virus. In some embodiments, the virus being neutralized by the disclosed method is human papillomavirus. In some embodiments, the virus being neutralized by the disclosed method is respiratory syncytial virus. In some embodiments, the virus being neutralized by the disclosed method is filovirus. In some embodiments, the virus being neutralized by the disclosed method is zaire ebolavirus. In some embodiments, the virus being neutralized by the disclosed method is sudan ebolavirus. In some embodiments, the virus being neutralized by the disclosed method is marburgvirus. In some embodiments, the virus being neutralized by the disclosed method is influenza virus.

Disclosed are methods of neutralizing infection of one or a plurality of viruses in a subject comprising administering to the subject any of the disclosed pharmaceutical compositions. In some embodiments, the viral infection being neutralized by the disclosed method is an infection of retroviridae. In some embodiments, the viral infection being neutralized by the disclosed method is an infection of flavivirus. In some embodiments, the viral infection being neutralized by the disclosed method is an infection of Nipah Virus. In some embodiments, the viral infection being neutralized by the disclosed method is an infection of West Nile virus. In some embodiments, the viral infection being neutralized by the disclosed method is an infection of human papillomavirus. In some embodiments, the viral infection being neutralized by the disclosed method is an infection of respiratory syncytial virus. In some embodiments, the viral infection being neutralized by the disclosed method is an infection of filovirus. In some embodiments, the viral infection being neutralized by the disclosed method is an infection of zaire ebolavirus. In some embodiments, the viral infection being neutralized by the disclosed method is an infection of sudan ebolavirus. In some embodiments, the viral infection being neutralized by the disclosed method is an infection of marburgvirus. In some embodiments, the viral infection being neutralized by the disclosed method is an infection of influenza virus.

Disclosed are methods of stimulating a therapeutically effective antigen-specific immune response against a virus in a mammal infected with the virus comprising administering any of the disclosed pharmaceutical compositions. In some embodiments, the disclosed method is against retroviridae. In some embodiments, the disclosed method is against flavivirus. In some embodiments, the disclosed method is against Nipah Virus. In some embodiments, the disclosed method is against West Nile virus. In some embodiments, the disclosed method is against human papillomavirus. In some embodiments, the disclosed method is against respiratory syncytial virus. In some embodiments, the disclosed method is against filovirus. In some embodiments, the disclosed method is against zaire ebolavirus. In some embodiments, the disclosed method is against sudan ebolavirus. In some embodiments, the disclosed method is against marburgvirus. In some embodiments, the disclosed method is against influenza virus.

Disclosed are methods of inducing expression of a self-assembling vaccine in a subject comprising administering any of the disclosed pharmaceutical compositions. Also disclosed are methods of treating a subject having a viral infection or susceptible to becoming infected with a virus comprising administering to the subject a therapeutically effective amount of any of the disclosed pharmaceutical compositions. In some embodiments, the viral infection is an infection of retroviridae. In some embodiments, the viral infection is an infection of flavivirus. In some embodiments, the viral infection is an infection of Nipah Virus. In some embodiments, the viral infection is an infection of West Nile virus. In some embodiments, the viral infection is an infection of human papillomavirus. In some embodiments, the viral infection is an infection of respiratory syncytial virus. In some embodiments, the viral infection is an infection of filovirus. In some embodiments, the viral infection is an infection of zaire ebolavirus. In some embodiments, the viral infection is an infection of sudan ebolavirus. In some embodiments, the viral infection is an infection of marburgvirus. In some embodiments, the viral infection is an infection of influenza virus.

In some embodiments, the administering can be accomplished by oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, intradermal or combinations thereof. In some embodiments, the above modes of action are accomplished by injection of the pharmaceutical compositions disclosed herein.

In some embodiments, the therapeutically effective dose can be from about 1 to about 30 micrograms of expressible nucleic acid sequence. In some embodiments, the therapeutically effective dose can be from about 0.001 micrograms of composition per kilogram of subject to about 0.050 micrograms per kilogram of subject.

In some embodiments, any of the disclosed methods can be free of activating any mannose-binding lectin or complement process.

In some embodiments, the subject can be a human. In some embodiments, the subject is diagnosed with or suspected of having a viral infection. For example, the subject can be diagnosed with or suspected of having an HIV-1 infection.

In some embodiments of the methods of inducing an immune response, the immune response can be an antigen-specific immune response. For example, the antigen-specific immune response can be an HIV-1 antigen immune response.

In some embodiments, the methods are free of administering any polypeptide directly to the subject.

In some embodiments, methods of inducing an immune response can include inducing a humoral or cellular immune response. A humoral immune response mainly refers to antibody production. A cellular immune response can include activation of CD4+ T-cells and activation CD8+ cells and associated cytotoxic activity. In one aspect, the present disclosure features a method of inducing an immune response in a subject, the method comprising administering to the subject in need thereof a pharmaceutically effective amount of any of the nucleic acid molecules of any one of the aspects or embodiments herein, or any one of the pharmaceutical compositions of any one of the aspects and embodiments herein. In one aspect, the present disclosure features a method of inducing a CD8+ T cell immune response in a subject, the method comprising administering to the subject in need thereof a pharmaceutically effective amount of any of the nucleic acid molecules of any one of the aspects or embodiments herein, or any one of the pharmaceutical compositions of any one of the aspects and embodiments herein.

In one aspect, the present disclosure features a method of enhancing an immune response in a subject, the method comprising administering to the subject in need thereof a pharmaceutically effective amount of any of the nucleic acid molecules of any one of the aspects or embodiments herein, or any one of the pharmaceutical compositions of any one of the aspects and embodiments herein.

In one aspect, the present disclosure features a method of enhancing a CD8+ T cell immune response in a subject, the method comprising administering to the subject in need thereof a pharmaceutically effective amount of any of the nucleic acid molecules of any one of the aspects or embodiments herein, or any one of the pharmaceutical compositions of any one of the aspects and embodiments herein.

In some embodiment, the subject has a viral infection. In some embodiments, the viral infection is an infection of retroviridae. In some embodiments, the viral infection is an infection of flavivirus. In some embodiments, the viral infection is an infection of Nipah Virus. In some embodiments, the viral infection is an infection of West Nile virus. In some embodiments, the viral infection is an infection of human papillomavirus. In some embodiments, the viral infection is an infection of respiratory syncytial virus. In some embodiments, the viral infection is an infection of filovirus. In some embodiments, the viral infection is an infection of zaire ebolavirus. In some embodiments, the viral infection is an infection of sudan ebolavirus. In some embodiments, the viral infection is an infection of marburgvirus. In some embodiments, the viral infection is an infection of influenza virus.

In some embodiments, the subject has previously been treated, and not responded to anti-viral therapy. In some embodiments, the nucleic acid molecule and/or expressible sequence is administered to the subject by electroporation.

The vaccine may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The vaccine may be administered by traditional syringes, needleless injection devices, “microprojectile bombardment gone guns”, or other physical methods such as electroporation (“EP”), “hydrodynamic method”, or ultrasound.

The plasmid of the vaccine may be delivered to the mammal by several well-known technologies including DNA injection (also referred to as DNA vaccination) with and without in vivo electroporation, liposome mediated, nanoparticle facilitated, recombinant vectors such as recombinant adenovirus, recombinant adenovirus associated virus and recombinant vaccinia. The consensus antigen may be delivered via DNA injection and along with in vivo electroporation.

The vaccine or pharmaceutical composition can be administered by electroporation. Administration of the vaccine via electroporation of the plasmids of the vaccine may be accomplished using electroporation devices that can be configured to deliver to a desired tissue of a mammal a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation can be accomplished using an in vivo electroporation device, for example CELLECTRA® EP system (Inovio Pharmaceuticals, Inc., Blue Bell, Pa.) or Elgen electroporator (Inovio Pharmaceuticals, Inc.) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the DNA vaccines of the present disclosure, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the DNA vaccines include those provided in co-pending and co-owned U.S. patent application Ser. No. 11/874,072, filed Oct. 17, 2007, which claims the benefit under 35 USC 119(e) to U.S. Provisional Applications Ser. No. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference in its entirety.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference in its entirety. The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to a method of injecting DNA are contemplated herein. The above-patents are incorporated by reference in their entireties.

Methods of preparing the nucleic acid sequences are disclosed. In some embodiments, plasmid sequences with one or more multiple dining sites my be purchased from commercially available vendors and the expressible nucleic acid sequences disclosed herein may be ligated into the plasmids after a digestion with a known restriction enzyme needed to cute the plasmid DNA. In another alternative embodiment, membrane-based purification methods disclosed herein offer reduced cost, high binding capacity, and high flow rates, resulting in a superior purification process. The purification process is further demonstrated to produce plasmid products substantially free of genomic DNA, RNA, protein, and endotoxin.

In some embodiments, all of the described aspects of the present disclosure are advantageously combined to provide an integrated process for preparing substantially purified cellular components of interest from cells in bioreactors. Again, the cells are most preferably plasmid-containing cells, and the cellular components of interest are most preferably plasmids. The substantially purified plasmids are suitable for various uses, including, but not limited to, gene therapy, plasmid-mediated therapy, as DNA vaccines for human, veterinary, or agricultural use, or for any other application that requires large quantities of purified plasmid. In this aspect, all of the advantages described for individual aspects of the present disclosure accrue to the complete, integrated process, providing a highly advantageous method that is rapid, scalable, and inexpensive. Enzymes and other animal-derived or biologically sourced products are avoided, as are carcinogenic, mutagenic, or otherwise toxic substances. Potentially flammable, explosive, or toxic organic solvents are similarly avoided.

One aspect of the present disclosure is an apparatus for isolating plasmid DNA from a suspension of cells having both plasmid DNA and genomic DNA. An embodiment of the apparatus comprises a first tank and second tank in fluid communication with a mixer. The first tank is used for holding the suspension cells and the second tank is used for holding a lysis solution. The suspension of cells from the first tank and the lysis solution from the second tank are both allowed to flow into the mixer forming a lysate mixture or lysate fluid. The mixer comprises a high shear, low residence-time mixing device with a residence time of equal to or less than about 1 second. In a preferred embodiment, the mixing device comprises a flow through, rotor/stator mixer or emulsifier having linear flow rates from about 0.1 L/min to about 20 L/min. The lysate-mixture flows from the mixer into a holding coil for a period of time sufficient to lyse the cells and forming a cell lysate suspension, wherein the lysate-mixture has resident time in the holding coil in a range of about 2-8 minutes with a continuous linear flow rate.

The cell lysate suspension is then allowed to flow into a bubble-mixer chamber for precipitation of cellular components from the plasmid DNA. In the bubble mixer chamber, the cell lysate suspension and a precipitation solution or a neutralization solution from a third tank are mixed together using gas bubbles, which forms a mixed gas suspension comprising a precipitate and an unclarified lysate or plasmid containing fluid. The precipitate of the mixed gas suspension is less dense than the plasmid containing fluid, which facilitates the separation of the precipitate from the plasmid containing fluid. The precipitate is removed from the mixed gas suspension to give a clarified lysate having the plasmid DNA, and the precipitate having cellular debris and genomic DNA.

In some embodiments, the bubble mixer-chamber comprises a closed vertical column with a top, a bottom, a first, and a second side with a vent proximal to the top of the column. A first inlet port of the bubble mixer-chamber is on the first side proximal to the bottom of the column and in fluid communication with the holding coil. A second inlet port of the bubble mixer-chamber is proximal to the bottom on a second side opposite of the first inlet port and in fluid communication with a third tank, wherein the third tank is used for holding a precipitation or a neutralization solution. A third inlet port of the bubble mixer-chamber is proximal to the bottom of the column and about in the middle of the first and second inlets and is in fluid communication with a gas source the third inlet entering the bubble-mixer-chamber. A preferred embodiment utilizes a sintered sparger inside the closed vertical column of the third inlet port. The outlet port exiting the bubble mixing chamber is proximal to the top of the closed vertical column. The outlet port is in fluid communication with a fourth tank, wherein the mixed gas suspension containing the plasmid DNA is allowed to flow from the bubble-mixer-chamber into the fourth tank. The fourth tank is used for separating the precipitate of the mixed gas suspension having a plasmid containing fluid, and can also include an impeller mixer sufficient to provide uniform mixing of fluid without disturbing the precipitate. A fifth tank is used for a holding the clarified lysate or clarified plasmid containing fluid. The clarified lysate is then filtered at least once. A first filter has a particle size limit of about 5-10 μm and the second filter has a cut of about 0.2 μm. Although gravity, pressure, vacuum, or a mixture thereof can be used for transporting: suspension of cells; lysis solutions; precipitation solutions; neutralization solutions; or mixed gas suspensions from any of the tanks to mixers, holding coils or different tanks, pumps are utilized in a preferred embodiments. In a more preferred embodiment, at least one pump having a linear flow rate from about 0.1 to about 1 ft/second is used.

In another specific embodiment, a Y-connector having a having a first bifurcated branch, a second bifurcated branch and an exit branch is used to contact the cell suspension and the lysis solutions before they enter the high shear, low residence-time mixing device. The first tank holding the cell suspension is in fluid communication with the first bifurcated branch of the Y-connector through the first pump and the second tank holding the lysis solution is in fluid communication with the second bifurcated branch of the Y-connector through the second pump. The high shear, low residence-time mixing device is in fluid communication with an exit branch of the Y-connector, wherein the first and second pumps provide a linear flow rate of about 0.1 to about 2 ft/second for a contacted fluid exiting the Y-connector.

Another specific aspect of the present disclosure is a method of substantially separating plasmid DNA and genomic DNA from a bacterial cell lysate. The method comprises: delivering a cell lysate into a chamber; delivering a precipitation fluid or a neutralization fluid into the chamber; mixing the cell lysate and the precipitation fluid or a neutralization fluid in the chamber with gas bubbles forming a gas mixed suspension, wherein the gas mixed suspension comprises the plasmid DNA in a fluid portion (i.e. an unclarified lysate) and the genomic DNA is in a precipitate that is less dense than the fluid portion; floating the precipitate on top of the fluid portion; removing the fluid portion from the precipitate forming a clarified lysate, whereby the plasmid DNA in the clarified lysate is substantially separated from genomic DNA in the precipitate. In preferred embodiments: the chamber is the bubble mixing chamber as described above; the lysing solution comprises an alkali, an acid, a detergent, an organic solvent, an enzyme, a chaotrope, or a denaturant; the precipitation fluid or the neutralization fluid comprises potassium acetate, ammonium acetate, or a mixture thereof; and the gas bubbles comprise compressed air or an inert gas. Additionally, the decanted-fluid portion containing the plasmid DNA is preferably further purified with one or more purification steps selected from a group consisting of: ion exchange, hydrophobic interaction, size exclusion, reverse phase purification, endotoxin depletion, affinity purification, adsorption to silica, glass, or polymeric materials, expanded bed chromatography, mixed mode chromatography, displacement chromatography, hydroxyapatite purification, selective precipitation, aqueous two-phase purification, DNA condensation, thiophilic purification, ion-pair purification, metal chelate purification, filtration through nitrocellulose, or ultrafiltration.

In some embodiments, a method for isolating a plasmid DNA from cells comprising: mixing a suspension of cells having the plasmid DNA and genomic DNA with a lysis solution in a high-shear-low-residence-time-mixing-device for a first period of time forming a cell lysate fluid; incubating the cell lysate fluid for a second period of time in a holding coil forming a cell lysate suspension; delivering the cell lysate suspension into a chamber; delivering a precipitation/neutralization fluid into the chamber; mixing the cell lysate suspension and the a precipitation/neutralization fluid in the chamber with gas bubbles forming a gas mixed suspension, wherein the gas mixed suspension comprises an unclarified lysate containing the plasmid DNA and a precipitate containing the genomic DNA, wherein the precipitate is less dense than the unclarified lysate; floating the precipitate on top of the unclarified lysate; removing the precipitate from the unclarified lysate forming a clarified lysate, whereby the plasmid DNA is substantially separated from genomic DNA; precipitating the plasmid DNA from the clarified lysate forming a precipitated plasmid DNA; and resuspending the precipitated plasmid DNA in an aqueous solution.

The disclosure also relates to a method of treating and/or preventing viral infection in a subject comprising administering to the subject a therapeutically and/or prophylactically effective amount (as applicable) of a pharmaceutical composition comprising at least one expressible nucleic acid sequence, the expressible nucleic acid sequence comprising in 5′ to 3′ orientation a first, second and third nucleic acid sequence; wherein the first nucleic acid sequence encodes a leader sequence, the second nucleic acid sequence encodes a self-assembling polypeptide, and the third nucleic acid sequence encodes a viral antigen. In some embodiments, the first, second and third nucleic acid sequences are contiguous. In some embodiments, the first, second, third nucleic acid sequence are non-contiguous and are separated by one or a plurality of other independently selectable nucleic acids encoding the same or different viral antigens. In some embodiments, the first, second, third nucleic acid sequence are non-contiguous and are separated by one or a plurality of other independently selectable nucleic acids encoding the same or different self-assembling peptides.

### F. Vaccines

Disclosed are vaccines comprising a first amino acid sequence comprising at least about 70%, 75%, 80%, 85%, 90%, 91%, 92%, 93%, 94%, 95%, 96%, 97%, 99% or 100% sequence identity to SEQ ID NO:7; and/or a second amino acid sequence comprising at least about 70%, 75%, 80%, 85%, 90%, 91%, 92%, 93%, 94%, 95%, 96%, 97%, 99% or 100% sequence identity to SEQ ID NO:9.

In some embodiments, the vaccines further comprise a linker fusing the first and second amino acid sequences. For example, the linker can be an amino acid sequence comprising at least about 70%, 75%, 80%, 85%, 90%, 91%, 92%, 93%, 94%, 95%, 96%, 97%, 99% or 100% sequence identity to SEQ ID NO:8 or any sequence identifier disclosed herein encoding a linker.

Also disclosed are DNA vaccines comprising an expressible nucleic acid sequence encoding a polypeptide comprising at least about 70%, 75%, 80%, 85%, 90%, 91%, 92%, 93%, 94%, 95%, 96%, 97%, 99% or 100% sequence identity to SEQ ID NO:10, SEQ ID NO:21, SEQ ID NO:25, SEQ ID NO:30, SEQ ID NO:35, SEQ ID NO:36, SEQ ID NO:38, SEQ ID NO:58, SEQ ID NO:60, SEQ ID NO:62, SEQ ID NO:64, SEQ ID NO:65, SEQ ID NO:66 or SEQ ID NO:67, or a pharmaceutically acceptable salt thereof. In some embodiments, the expressible nucleic acid sequence of the disclosed DNA vaccines comprises at least about 70%, 75%, 80%, 85%, 90%, 91%, 92%, 93%, 94%, 95%, 96%, 97%, 99% or 100% sequence identity to SEQ ID NO:5, SEQ ID NO:20, SEQ ID NO:24, SEQ ID NO:28, SEQ ID NO:33, SEQ ID NO:34, SEQ ID NO:37, SEQ ID NO:57, SEQ ID NO:59, SEQ ID NO:61 or SEQ ID NO:63, or a pharmaceutically acceptable salt thereof. In some embodiments, the disclosed DNA vaccine further comprises a pharmaceutically acceptable excipient. In some embodiments, the pharmaceutically acceptable excipient is an adjuvant.

### G. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising any of the elements of the disclosed nucleic acid compositions. For example, disclosed are kits comprising nucleic acid sequences comprising a leader sequence, a linker sequence, a nucleic acid sequence encoding a self-assembling polypeptide, and/or a nucleic acid sequence encoding a viral antigen. In some embodiments, the kits can further comprise a plasmid backbone.

### EXAMPLES

### Example 1: Production of and Experimentation with Plasmids Expressing Self-Assembling Nanoparticles

### Immunization

For DNA-based immunization, 6-8 week old female C57BL/6 or BALB/c mice purchased from Jackson laboratory were immunized once or twice with 2, 10, and 25 ug of DNA-plasmid encoding IgE-GLT1-NP or IgE-GLT1 via intramuscular injections into the tibialis anterior muscles (over two sites), followed by intramuscular electroporation with Cellectra 3P device. MBL knockout mice (B6.129S4-Mbl1tm1Kata Mbl2tm1Kata/J) and CR2 knockout mice (B6.12957(NOD)-Cr2tm1Hmo/J) purchased from Jackson Laboratory were immunized in the same fashion. For protein-based immunization, 6-8 week old female C57BL/6, MBL knockout and CR2 knockout mice were immunized subcutaneously over two sites with bug of recombinant IgE-GLT1-NP protein coformulated in Sigma adjuvant system.

GT8-Binding ELISA

Corning 96-well half area plates were coated at room temperature for 6 hours with 1 ug/mL MonoRab anti-His antibody, followed by overnight blocking with solution containing 1× PBS, 5% skim milk, 10% goat serum, 1% BSA, 1% FBS, and 0.2% Tween-20. The plates were then incubated with 2 ug/mL of his-tagged GT8 monomer at room temperature for 2 hours, followed by addition of mice sera serially diluted with PBS with 1% FBS and 0.1% Tween and incubation at 37 C for 2 hours. The plates were then incubated at room temperature for 1 hour with anti-mouse IgG H+L HRP (Bethyl) at 1:20,000 dilution, followed by addition of TMB substrates. Absorbance at 450 nm and 570 nm were recorded with BioTEK plate reader.

VRC01 Competition ELISA

The plates were coated, and blocked, followed by addition with GT8-his as described in the last section. Serially diluted mice sera were then incubated with the plates at 37 C for 1 hour, followed by addition of purified VRC01 antibody (NIH AIDS Reagent) for an additional 1 hour at room temperature. The plates were then incubated with anti-human Fc (cross-adsorbed against rabbits) (Jackson laboratory) at 1:10,000 dilution for 1 hour, followed by addition of TMB substrate for detection.

MBL binding ELISA The plates were coated with 5 ug/mL recombinant mouse MBL protein (R&D system) in 0.1M CaCl₂ at room temperature for 6 hours, followed by blocking with 1% BSA in 0.1M CaCl₂ in PBS overnight at 4 C. Transfection supernatant or muscle homogenates containing IgE-GLT1 or IgE-GLT1-NP were then added to the plates for 2 hour incubation at 37 C, followed by Week 5 sera of BALB/c mice immunized twice with 25 ug DNA-encoded IgE-GLT1-NP. The plates were then incubated with anti-mouse IgG H+L HRP (Bethyl) at 1:20,000 dilution, followed by addition of TMB substrates.

VRC01 Binding ELISA

ELISA format as described in the MBL binding ELISA section except that the recombinant MBL used in the coating step is replaced by 5 ug/mL of VRC01 (NIH AIDS Reagent).

Immunofluorescence

For lymph node staining, 6 days after BALB/c mice were immunized with DNA encoding IgE-GLT1 or IgE-GLT1-NP, tibialis anterior muscles of the mice were injected with 5 ug of anti-mouse CD35 BV421 (BD-Bioscience) for in situ labelling of follicular dendritic cells. Ipsilateral inguinal lymph nodes from the mice were harvested the next day and preserved in O.C.T medium for cryosectioning. The sections were fixed with formaldehyde, permeablized with 0.5% Triton X-100 then blocked in 3% BSA/PBS for 1 hour at room temperature, followed by overnight staining with 6 ug/mL VRC01. The sections were then washed, and stained with anti-human Alexa Fluor 488 antibody and imaged with Leica SP5 confocal microscopes.

For muscle staining, 4 days after BALB/c mice were immunized with DNA encoding IgE-GLT1 or IgE-GLT1-NP, tibialis anterior muscles of the mice were harvested the and preserved in O.C.T medium for cryosectioning. The sections were then blocked in 3% BSA/PBS for 1 hour at room temperature, followed by overnight staining with 6 ug/mL VRC01. The sections were then washed, and stained with anti-human Alexa Fluor 488 antibody, counterstained with 0.5 ug/mL DAPI and imaged with Leica SP5 confocal microscopes.

For transfected cells, HEK293T cells were cultured in poly-lysine coated glass chambers overnight, and then transfected with DNA encoding IgE-GLT1 or IgE-GLT1-NP with GeneJammer (Agilent). The cells were harvested 48 hours post transfection, fixed, permeabilized, blocked and stained as in the section describing muscle immunofluorescence staining.

Immunohistochemistry

For immunohistochemistry staining of muscle sections, BALB/C mice were immunized with DNA-encoding IgE-GLT1 or IgE-GLT1-NP. Transfected muscles were harvested 7 days post immunization, cryosectioned, fixed, permeabilized, and block as described in the Immunofluorescence section. The muscle sections were then stained with goat anti-mouse MBL at 1:200 dilution (R&D system) overnight, and then with secondary Rabbit anti-goat (H+L) HRP conjugated at 1:500 dilution (BioRad) and DAB substrates for development.

Electron Microscopy

Tibialis anterior muscles from BALB/c mice immunized with DNA-encoding IgE-GLT1-NP or naïve mice were collected 7 days post immunization (for IgE-GLT1-NP mice). The muscles were then fixed in 2.5% glutaraldehyde, serially dehydrated in acetone/ethanol solvents, and then embedded in epoxy and LR white resin. The resin was then sectioned to a thickness of 70 nm and deposited onto a metal grid, blocked overnight in 3% BSA/PBS, followed by staining with 60 ug/mL VRC01 (diluted in 3% BSA/PBS) overnight, and with 1:200 anti-human 6 nm gold nanoparticles (Jackson Immunoresearch) for 1 hour. The sections were then washed with 0.1% Tween in PBS, and water, followed by post-staining fixation with 2.5% glutaraldehyde in PBS for 5 minutes at room temperature followed by staining with 2% Uranyl acetate for 1 hour. The grids were subsequently imaged with JEOL JEM 1010 transmission electron microscope.

EliSpot Assay

Spleens from immunized mice were collected 5 weeks post the first immunization, and homogenized into single cell suspension with a tissue stomacher in 10% FBS/1% Penicillin-streptomycin in RPMI 1640. 200,000 cells were then plated in each well in the mouse IFN-γ EliSpot plates (MabTech), followed by addition peptide pools that span both the lumazine synthase and GP120 domains at 5 ug/mL of final concentration for each peptide. The cells were then stimulated at 37 C for 16-18 hours, followed by development according to the manufacturer's instructions. Spots for each well were then imaged and counted with ImmunoSpot Macro Analyzer.

Intracellular Cytokine Staining

Single cell suspension from spleens of immunized animals were prepared as described in the previous section, and stimulated with 5 ug/mL of peptides spanning both the lumazine synthase and GP120 domains for 5 hours at 37 C in the presence of 1:500 protein transport inhibitor (ThermoFisher) and anti-mouse CD107a-FITC. The cells were then incubated with live/dead for 10 min at room temperature, surface stains (anti-mouse CD4 BV510, anti-mouse CD8 APC-Cy7, anti-mouse CD62L BV711 and anti-mouse CD44 AF700) (BD-Biosciences) at room temperature for 30 minutes. The cells were then fixed and permeabilized according to manufacturer's instructions for BD Cytoperm Cytofix kit and stained with intracellular stains (anti-mouse IL-2 PE-Cy7, anti-mouse IFN-γ APC, anti-mouse CD3e PE-Cy5 and anti-mouse TNFα BV605) at 4 C for 1 hour. The cells were subsequently analyzed with LSR II 18-color flow cytometer.

Immunoblotting

Tibialis anterior muscles of immunized animals were harvested and homogenized in T-PER extraction buffer (ThermoFisher) and protease inhibitor (Roche). Supernatant of Expi293F cells transfected with DNA-encoding IgE-GTL1-NP or muscle homogenates from mice immunized with DNA-encoded IgE-GTL1-NP were loaded onto 4-12% SDS Bis-Tris Gel or 3-12% Native Bis-Tris Gel for electrophoresis. Proteins were subsequently transferred to PVDF membrane from the gels, and stained with 3 ug/mL of VRC01 in Odyssey Blocking Buffer (0.1% Tween) overnight at 4 C, and 1:10,000 IRDye 800CW goat anti-human IgG (LI-COR Biosciences) in Odyssey Blocking Buffer (0.1% Tween, 0.1% SDS) at room temperature for 1 hour, and then scanned with with LI-COR Odyssey CLx0.

Production of His-Tagged GT8 and recombinant IgE-GLT1-NP HEK293F cells were transfected with DNA-encoding IgE-GLT1-NP or His-Tagged GT8 with PEI/OPTI-MEM and harvested 6 days post-transfection. Transfection supernatant was first purified with affinity chromatography using the AKTA pure 25 system (and using the IMAC Nickel column for His-tagged GT8 and GNL Lectin beads for IgE-GLT1-NP). The eluate fractions from the affinity purification were pooled, concentrated and dialyzed into 1×PBs buffer before being loaded onto the SEC column and then with size exclusion chromatography (Superdex 200 Increase 10/300 GL column for His-tagged GT8 and Superose 6 Increase 10/300 GL for IgE-GLT1-NP). Identified eluate fractions were then collected and concentrated to 1 mg/mL in PBS.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Jardine J G, Kulp D W, Havenar-Daughton C, et al. HIV-1 broadly neutralizing antibody precursor B cells revealed by germline-targeting immunogen. Science 2016; 351(6280): 1458-63.
2. Steichen J M, Kulp D W, Tokatlian T, et al. HIV Vaccine Design to Target Germline Precursors of Glycan-Dependent Broadly Neutralizing Antibodies. Immunity 2016; 45(3): 483-96.
3. McGuire A T, Hoot S, Dreyer A M, et al. Engineering HIV envelope protein to activate germline B cell receptors of broadly neutralizing anti-CD4 binding site antibodies. J Exp Med 2013; 210(4): 655-63.
4. Medina-Ramirez M, Garces F, Escolano A, et al. Design and crystal structure of a native-like HIV-1 envelope trimer that engages multiple broadly neutralizing antibody precursors in vivo. J Exp Med 2017; 214(9): 2573-90.
5. Kulp D W, Steichen J M, Pauthner M, et al. Structure-based design of native-like HIV-1 envelope trimers to silence non-neutralizing epitopes and eliminate CD4 binding. Nat Commun 2017; 8(1): 1655.
6. He L, Kumar S, Allen J D, et al. HIV-1 vaccine design through minimizing envelope metastability. Sci Adv 2018; 4(11): eaau6769.
7. Rutten L, Lai Y T, Blokland S, et al. A Universal Approach to Optimize the Folding and Stability of Prefusion-Closed HIV-1 Envelope Trimers. Cell Rep 2018; 23(2): 584-95.
8. Gregory A E, Titball R, Williamson D. Vaccine delivery using nanoparticles. Front Cell Infect Microbiol 2013; 3: 13.
9. Zhao L, Seth A, Wibowo N, et al. Nanoparticle vaccines. Vaccine 2014; 32(3): 327-37.
10. Speiser D E, Schwarz K, Baumgaertner P, et al. Memory and effector CD8 T-cell responses after nanoparticle vaccination of melanoma patients. J Immunother 2010; 33(8): 848-58.
11. Sliepen K, Ozorowski G, Burger J A, et al. Presenting native-like HIV-1 envelope trimers on ferritin nanoparticles improves their immunogenicity. Retrovirology 2015; 12: 82.
12. Chattopadhyay S, Chen J Y, Chen H W, Hu C J. Nanoparticle Vaccines Adopting Virus-like Features for Enhanced Immune Potentiation. Nanotheranostics 2017; 1(3): 244-60.
13. Desai N. Challenges in development of nanoparticle-based therapeutics. AAPS J 2012; 14(2): 282-95.
14. Pattenden L K, Middelberg A P, Niebert M, Lipin D I. Towards the preparative and large-scale precision manufacture of virus-like particles. Trends Biotechnol 2005; 23(10): 523-9.
15. Lua L H, Connors N K, Sainsbury F, Chuan Y P, Wibowo N, Middelberg A P. Bioengineering virus-like particles as vaccines. Biotechnol Bioeng 2014; 111(3): 425-40.
16. Kanekiyo M, Wei C J, Yassine H M, et al. Self-assembling influenza nanoparticle vaccines elicit broadly neutralizing H1N1 antibodies. Nature 2013; 499(7456): 102-6.

1. (canceled)

2. A composition comprising an expressible nucleic acid sequence comprising:
(i) a nucleic acid sequence encoding a self-assembling polypeptide; and
(iii) a nucleic acid sequence encoding at least one viral antigen.

3. The composition of claim 2, wherein the nucleic acid sequence encoding a self-assembling polypeptide comprises at least about 70% sequence identity to one or a combination of: SEQ ID NO: 2, SEQ ID NO: 13, SEQ ID NO: 14, and SEQ ID NO: 15.

4. The composition of claim 3 wherein the self-assembling polypeptide comprises at least about 70% sequence identity to one or a combination of: SEQ ID NO:7, SEQ ID NO:23, SEQ ID NO:31, and SEQ ID NO:26.

5. The composition of claim 2, further comprising a nucleic acid sequence encoding a leader peptide, wherein the expressible nucleic acid sequence is operably linked to one or a plurality of regulatory sequences.

6. The composition of claim 2, the composition further comprising a nucleic acid molecule, wherein the expressible nucleic acid sequence is in the nucleic acid molecule.

7. The composition of claim 6, wherein the nucleic acid molecule is a plasmid.

8. The composition of claim 2, wherein the viral antigen is an antigen from human immunodeficiency virus-1 (HIV-1).

9. The composition of claim 8 wherein the viral antigen comprises at least about 70% sequence identity to SEQ ID NO: 9 or a pharmaceutically acceptable salt thereof.

10. The composition of claim 2, wherein the expressible nucleic acid sequence further comprises at least one nucleic acid sequence encoding a linker.

11. The composition of claim 10, wherein the at least one nucleic acid sequence encoding a linker comprises at least about 70% sequence identity to SEQ ID NO:3 or a pharmaceutically acceptable salt thereof.

12. The composition of claim 2, wherein the nucleotide sequence encoding a self-assembling polypeptide comprises at least about 70% sequence identity to SEQ ID NO:2 or encoding a polypeptide comprising at least about 70% sequence identity to SEQ ID NO:7, or a pharmaceutically acceptable salt thereof.

13. The composition of claim 7, wherein the plasmid comprises an expressible nucleic acid sequence comprising at least about 70% sequence identity to SEQ ID NO:5, SEQ ID NO:11, SEQ ID NO:20, SEQ ID NO:24, SEQ ID NO:28, SEQ ID NO:33, SEQ ID NO:34, SEQ ID NO:37, SEQ ID NO:57, SEQ ID NO:59, SEQ ID NO:61 or SEQ ID NO:63, or a pharmaceutically acceptable salt thereof, or wherein the plasmid comprises an expressible nucleic acid sequence encoding a polypeptide comprising at least about 70% sequence identity to SEQ ID NO:10, SEQ ID NO:12, SEQ ID NO:21, SEQ ID NO:25, SEQ ID NO:30, SEQ ID NO:35, SEQ ID NO:36, SEQ ID NO:38, SEQ ID NO:58, SEQ ID NO:60, SEQ ID NO:62 or SEQ ID NO:64, or a pharmaceutically acceptable salt thereof.

14. A pharmaceutical composition comprising: (i) the composition of claim 2; and (ii) a pharmaceutically acceptable carrier.

15-16. (canceled)

17. A method of treating or preventing a viral infection in a subject comprising administering to the subject a therapeutically effective amount of the pharmaceutical composition of claim 14.

18. The method of claim 17, wherein the administering comprises oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, or intraarticular administration.

19. The method of claim 17, wherein the therapeutically effective amount is from about 20 to about 2000 micrograms of the expressible nucleic acid sequence.

20. The method of claim 17, wherein the method is free of activating any mannose-binding lectin or complement process.

21. The method claim 17, wherein the subject is a human.

22. The method of claim 17, wherein the therapeutically effective amount is from about 0.3 micrograms of composition per kilogram of subject to about 30 micrograms per kilogram of subject.

23. A method of inducing an immune response in a subject comprising administering to the subject a therapeutically effective amount of the pharmaceutical composition of claim 14.

24. The method of claim 23, wherein the administering comprises oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, or intraarticular administration.

25. The method of claim 23, wherein from about 1 to about 2000 micrograms of the expressible nucleic acid sequence is administered.

26. The method of claim 23, wherein the method is free of activating any mannose-binding lectin or complement process.

27. The method of claim 23, wherein the subject is a human.

28. The method of claim 23, wherein the therapeutically effective amount is from about 0.3 micrograms of composition per kilogram of subject to about 30 micrograms per kilogram of subject.

29. The method of claim 23, wherein the immune response comprises an antigen-specific immune response.

30. The method of claim 23, wherein the subject is diagnosed with or suspected of having an HIV-1 infection.

31. The method of claim 23, wherein the immune response comprises an antigen-specific immune response against an HIV-1 antigen.

32-41. (canceled)

42. A vaccine comprising:
(i) the composition of claim 7;
(ii) a pharmaceutically acceptable carrier.

43. (canceled)

44. The vaccine of claim 43, wherein the expressible nucleic acid sequence further comprises (iv) a nucleic acid sequence encoding a linker, wherein the nucleic acid sequence encoding a linker comprises a sequence having at least about 70% sequence identity to SEQ ID NO:3 or the linker is an amino acid sequence comprising at least about 70% sequence identity to SEQ ID NO:8.

45-51. (canceled)

52. The composition of claim 2, wherein the at least one viral antigen comprises a retroviridae antigen, a flavivirus antigen, a Nipah Virus antigen, a human papillomavirus antigen, a respiratory syncytial virus antigen, a Filovirus antigen, or an influenza virus antigen.

53-99. (canceled)

100. The composition of claim 7, wherein the plasmid comprises SEQ ID NO: 56.
